(19) United States
(12) Patent Application Publication
(10) Pub. No.: US 2025/0144003 A1
(43) Pub. Date: May 8, 2025

(54) POLYHYDROXYALKANOATE COPOLYMER BEARING AN ACETOACETATE GROUP, COMPOSITION CONTAINING SAME AND COSMETIC USE THEREOF

(71) Applicant: L'OREAL, Paris (FR)

(72) Inventors: Laurent SABATIE, Aulnay-Sous-Bois (FR); Etienne SAVONNET, Aulnay-Sous-Bois (FR)

(73) Assignee: L'OREAL, Paris (FR)

(21) Appl. No.: 18/706,694

(22) PCT Filed: Dec. 19, 2022
(86) PCT No.: PCT/EP2022/086800

§ 371 Date: May 1, 2024

(30) Foreign Application Priority Data
Dec. 20, 2021 (FR) FR2114050

(51) Int. Cl. A61K 8/85; A61Q 1/02; A61Q 5/10; C08G 63/06; C08G 63/91

(52) U.S. Cl. CPC A61K 8/85; A61Q 1/02; A61Q 5/10; C08G 63/06; C08G 63/912; A61K 2800/43; A61K 2800/432; A61K 2800/884

(57) ABSTRACT

The present invention relates to polyhydroxyalkanoate (PHA) copolymers bearing acetoacetate group(s) and derivative(s), to a composition comprising same, and also to a process for treating keratin materials using such PHAs, notably for making up the skin or for colouring or styling keratin fibres, preferably the hair.

Thus the main subject of the present invention is a) a polyhydroxyalkanoate (PHA) copolymer which contains, several units (A) below, and also the optical or geometrical isomers thereof, the organic or mineral acid or base salts thereof, and the solvates thereof such as hydrates:

—[—O—CH(R¹)—CH₂—C(O)—]— unit (A)

in which polymer units (A):
R¹ represents a saturated or unsaturated, linear or branched, non-cyclic hydrocarbon-based chain, or a saturated or unsaturated non-aromatic cyclic hydrocarbon-based chain, comprising from 3 to 30 carbon atoms, said hydrocarbon-based chain being substituted with one or more groups chosen from:
   A) R^{3′}—C(X)—C(R⁴)(R⁵)—C(X′)—[Y]ₙ—* and B) R^{3′}—C(X)—C(—[Y]ₙ—*)(R⁴)—C(X)—R⁶ with R^{3′}, R⁴, R⁵ and R⁶ as defined in the description.

The present invention relates to polyhydroxyalkanoate (PHA) copolymers bearing acetoacetate group(s) and derivative(s), to a composition comprising same, and also to a process for treating keratin materials using such PHAs, notably for making up the skin or for colouring or styling keratin fibres, preferably the hair.

It is known practice to use, in cosmetics, film-forming polymers which can be conveyed in organic media, such as hydrocarbon-based oils. Polymers are notably used as film-forming agents in makeup products such as mascaras, eyeliners, eyeshadows or lipsticks.

FR-A-2964663 describes a cosmetic composition comprising pigments coated with a C₃-C₂₁ polyhydroxyalkanoate, such as poly(hydroxybutyrate-co-hydroxyvalerate).

WO 2011/154508 describes a cosmetic composition comprising a 4-carboxy-2-pyrrolidinone ester derivative and a film-forming polymer which may be a polyhydroxyalkanoate, such as polyhydroxybutyrate, polyhydroxyvalerate and polyhydroxybutyrate-co-polyhydroxyvalerate.

The document US-A-2015/274972 describes a cosmetic composition comprising a cosmetic composition comprising a thermoplastic resin, such as a polyhydroxyalkanoate, in aqueous dispersion and a silicone elastomer.

The majority of the polyhydroxyalkanoate copolymers are polymers derived from the polycondensation of polymeric repeating units that are for the most part identical and derived from the same carbon source or substrate. These documents do not describe the cosmetic use of copolymers derived from polycondensation using an aliphatic substrate or first carbon source including one or more acetoacetate groups and derivatives as defined hereinbelow, and at least a second substrate different from the first, comprising one or more (un)saturated hydrocarbon-based groups. There is thus a need for polyhydroxyalkanoate copolymers which are lipophilic or soluble in a fatty phase.

Furthermore, there is a need for a composition comprising PHAs bearing diverse acetoacetate functionalization or which are functionalizable with lipophilic or non-lipophilic active agents, which could make them active and soluble in a fatty phase, or which are more active notably in the presence of crosslinking agent(s). This makes it possible to obtain a film on keratin materials which has good cosmetic properties, notably good resistance to oils and to sebum, and also to be able to modify the gloss or the mattness.

The Applicant has discovered that PHA copolymers bearing acetoacetate group(s) and derivative(s), as defined below, may be readily used in fatty media, thus making it possible to obtain homogeneous compositions. The composition shows good stability, notably after storage for one month at room temperature (25° C.). PHA copolymers bearing acetoacetate group(s) and derivative(s) as defined below, notably after their application to keratin materials, particularly in the presence of crosslinking agent(s) b), make it possible to obtain a film having good cosmetic properties and good resistance to oils (notably olive oil) and to sebum, and also a matt or glossy appearance. In addition, the residual film does not have a tacky feel.

Thus the main subject of the present invention is a) a polyhydroxyalkanoate (PHA) copolymer which contains, several units (A) below, and also the optical or geometrical isomers thereof, the organic or mineral acid or base salts thereof, and the solvates thereof such as hydrates:

—[—O—CH(R¹)—CH₂—C(O)—]— unit (A)

in which polymer units (A):
R¹ represents a saturated or unsaturated, linear or branched, non-cyclic hydrocarbon-based chain, or a saturated or unsaturated non-aromatic cyclic hydrocarbon-based chain, comprising from 3 to 30 carbon atoms; preferably, the hydrocarbon-based chain is chosen from i) linear or branched (C₅-C₂₃)alkyl, ii) linear or branched (C₆-C₂₃)alkenyl, iii) linear or branched (C₆-C₂₃)alkynyl, iii) preferably the hydrocarbon-based chain is non-cyclic and linear;
said hydrocarbon-based chain being:
   substituted with one or more groups chosen from:
      A) R^{3′}—C(X)—C(R⁴)(R⁵)—C(X′)—[Y]ₙ—* and B) R^{3′}—C(X)—C(—[Y]ₙ—*)(R⁴)—C(X)—R⁶ with:
R^{3′} and R⁶, which may be identical or different, representing a group chosen from optionally substituted (C₁-C₆)alkyl, optionally substituted (C₁-C₆)alkoxy, optionally substituted (C₁-C₆)alkylthio, optionally substituted (di)(C₁-C₆)(alkyl)amino, (hetero)aryl, (hetero)cycloalkyl, (hetero)aryloxy, (hetero)cycloalkyloxy, (hetero)arylthio, (hetero)cycloalkylthio, (hetero)arylamino, (hetero)cycloalkylamino; preferably representing a group chosen from (C₁-C₆)alkyl such as methyl, (C₁-C₆)alkoxy such as ethoxy, more preferentially (C₁-C₄)alkyl such as methyl;
R⁴ and R⁵, which may be identical or different, represent a hydrogen atom or a group chosen from (C₁-C₆)alkyl; preferably, R⁴ and R⁵ are identical, and particularly represent a hydrogen atom;
n is 0 or 1;
X and X′, which may be identical or different, represent an oxygen or sulfur atom or a group N—Rₐ with Rₐ representing a hydrogen atom or a (C₁-C₄)alkyl group; preferably, X and X′ are identical and particularly represent an oxygen atom;
Y represents a heteroatom chosen from O, S, N—Rₐ with Rₐ as defined previously; preferably, Y represents an oxygen atom;
* represents the point of attachment of group A) or 1) connected to the remainder of the PHA copolymer(s); and
the radicals R¹ also possibly being:
   substituted with one or more atoms or groups chosen from: a) halogen such as chlorine or bromine, b) hydroxyl, c) thiol, d) (di)(C₁-C₄)(alkyl)amino, e) (thio)carboxy, f) (thio)carboxamide —C(O)—N(Rₐ)₂ or C(S)—N(Rₐ)₂, g) cyano, h) iso(thio)cyanate, i) (hetero)aryl such as phenyl or furyl, and j) (hetero)cycloalkyl, k) cosmetic active agent; I) R—X with R representing a group chosen from α) cycloalkyl such as cyclohexyl, β) heterocycloalkyl such as sugar, preferably monosaccharide such as glucose, γ) (hetero)aryl such as phenyl, and m) thiosulfate; X representing a′) O, S, N(R′ₐ) or Si(R′_{b})(R′_{c}), b′) S(O)ᵣ, or (thio)carbonyl, c′) or combinations of a′) with b′) such as (thio)ester, (thio)amide, (thio)urea or sulfonamide; R′ₐ representing a hydrogen atom, or a (C₁-C₄)alkyl group or an aryl(C₁-C₄)alkyl group such as benzyl; preferably, Rₐ represents a hydrogen atom; R′_{b} and R′_{c}, which may be identical or different, represent a (C₁-C₄)alkyl or (C₁-C₄)alkoxy group, particularly only one substituent; preferably chosen from b) halogen, and j) such as epoxide; and/or
   optionally interrupted with one or more a′) heteroatoms such as O, S, N(Rₐ) and Si(R_{b})(R_{c}), b′) S(O)ᵣ, (thio)carbonyl, c′) or combinations of a′) with b′) such as (thio)ester, (thio)amide, (thio)urea, sulfonamide, preferably ester —O—C(O)— or —C(O)—O— with r being equal to 1 or 2, Rₐ being as defined previously, preferably Rₐ represents a hydrogen atom, R_{b} and R_{c} being as defined previously;

Another subject of the invention is a process for preparing PHA copolymers a) as defined previously.

Another subject of the invention is a preferably cosmetic composition, comprising a) one or more PHAs as defined previously, b) optionally one or more crosslinking agents; and c) optionally one or more fatty substances, which are preferably liquid at 25° C. and at atmospheric pressure. Another object of the invention is the cosmetic use of a) one or more PHA copolymers as defined previously, b) optionally one or more crosslinking agents as defined previously, and c) optionally one or more fatty substances as defined previously.

Another subject of the invention is a process for treating keratin materials, preferably α) keratin fibres, notably human keratin fibres such as the hair, or β) human skin, in particular the lips, using a) one or more PHA copolymers as defined previously and optionally b) one or more crosslinking agents as defined previously, and optionally c) one or more fatty substances as defined previously, a), b) and c) being applied together or separately.

More particularly, a subject of the invention is a non-therapeutic cosmetic process for treating keratin materials, comprising the application to the keratin materials of a PHA as defined previously or of a composition as defined previously. The treatment process is in particular a process for caring for or making up or colouring keratin materials.

A subject of the invention is also a device with several separate compartments (kit) comprising:
in a first compartment: a composition (Al) comprising:
one or more PHAs as defined previously; and
optionally one or more fatty substances, which are preferably liquid at 25° C. and at atmospheric pressure; and/or
optionally one or more colouring agents chosen from direct dyes, oxidation dyes, pigments and mixtures thereof; and
in a second compartment separate from the first: a composition (B1) comprising:
one or more crosslinking agents.

For the purposes of the present invention and unless otherwise indicated:
the term “cosmetic active agent” means the radical of an organic or organosilicon compound which can be integrated into a cosmetic composition to give an effect on keratin materials, whether this effect is immediate or provided by repeated applications. As examples of cosmetic active agents, mention may be made of coloured or uncoloured, fluorescent or non-fluorescent chromophores such as those derived from optical brighteners, or chromophores derived from UVA and/or UVB screening agents, anti-ageing active agents or active agents intended for providing a benefit to the skin such as active agents having action on the barrier function, deodorant active agents other than mineral particles, antiperspirant active agents other than mineral particles, desquamating active agents, antioxidant active agents, moisturizing active agents, sebum-regulating active agents, active agents intended for limiting the sheen of the skin, active agents intended for combating the effects of pollution, antimicrobial or bactericidal active agents, antidandruff active agents, and fragrances.
The term “(hetero)aryl” means aryl or heteroaryl groups;
The term “(hetero)cycloalkyl” means cycloalkyl or heterocycloalkyl groups;
The “aryl” or “heteroaryl” radicals or the aryl or heteroaryl part of a radical may be substituted with at least one substituent borne by a carbon atom, chosen from:
   a C₁-C₆ and preferably C₁-C₄ alkyl radical;
   a halogen atom such as chlorine, fluorine or bromine;
   a hydroxyl group;
   a C₁-C₂ alkoxy radical; a C₂-C₄ (poly)hydroxyalkoxy radical;
   an amino radical;
   an amino radical substituted with one or two identical or different C₁-C₆ and preferably C₁-C₄ alkyl radicals;
   an acylamino radical (—NR—COR′) in which the radical R is a hydrogen atom;
   a C₁-C₄ alkyl radical and the radical R′ is a C₁-C₄ alkyl radical; a carbamoyl radical ((R)₂N—CO—) in which the radicals R, which may be identical or different, represent a hydrogen atom or a C₁-C₄ alkyl radical;
   an alkylsulfonylamino radical (R′SO₂—NR—) in which the radical R represents a hydrogen atom or a C₁-C₄ alkyl radical and the radical R′ represents a C₁-C₄ alkyl radical, or a phenyl radical;
   an aminosulfonyl radical ((R)₂N—S(O)₂—) in which the radicals R, which may be identical or different, represent a hydrogen atom or a C₁-C₄ alkyl radical;
   a carboxylic radical in acid or salified (preferably with an alkali metal or a substituted or unsubstituted ammonium) form;
   a cyano group (CN);
   a polyhalo(C₁-C₄)alkyl group, preferentially trifluoromethyl (CF₃);
the cyclic or heterocyclic part of a non-aromatic radical may be substituted with at least one substituent borne by a carbon atom, chosen from the groups:
   hydroxyl;
   C₁-C₄ alkoxy, C₂-C₄ (poly)hydroxyalkoxy;
   alkylcarbonylamino (RCO—NR′—), in which the radical R′ is a hydrogen atom or a C₁-C₄ alkyl radical and the radical R is a C₁-C₂ alkyl radical or an amino radical substituted with one or two identical or different C₁-C₄ alkyl groups;
   alkylcarbonyloxy (RCO—O—), in which the radical R is a C₁-C₄ alkyl radical or an amino radical substituted with one or two identical or different C₁-C₄ alkyl groups;
   alkoxycarbonyl ((RO—CO—) in which the radical R is a C₁-C₄ alkyl radical or an amino radical substituted with one or two identical or different C₁-C₄ alkyl groups;
a cyclic or heterocyclic radical, or a non-aromatic part of an aryl or heteroaryl radical, may also be substituted with one or more oxo groups;
a hydrocarbon-based chain is unsaturated when it includes one or more double bonds and/or one or more triple bonds;
an “aryl” radical represents a monocyclic or fused or non-fused polycyclic hydrocarbon-based group comprising from 6 to 22 carbon atoms, and at least one ring of which is aromatic; preferentially, the aryl radical is a phenyl, biphenyl, naphthyl, indenyl, anthracenyl or tetrahydronaphthyl;
a “heteroaryl” radical represents a monocyclic or fused or non-fused polycyclic, 5- to 22-membered group, comprising from 1 to 6 heteroatoms chosen from nitrogen, oxygen, sulfur and selenium atoms, and at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridyl, tetrazolyl, dihydrothiazolyl, imidazopyridyl, imidazolyl, indolyl, isoquinolyl, naphthoimidazolyl, naphthooxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridyl, thiazoylimidazolyl, thiopyrylyl, triazolyl and xanthylyl;
a “cyclic” or “cycloalkyl” radical is a monocyclic or fused or non-fused polycyclic, non-aromatic cyclic hydrocarbon-based radical containing from 5 to 22 carbon atoms, which may include one or more unsaturations; the cycloalkyl is preferably a cyclohexyl group;
a “heterocyclic” or “heterocycloalkyl” radical is a monocyclic or fused or non-fused polycyclic 3- to 9-membered non-aromatic cyclic radical, including from 1 to 4 heteroatoms chosen from nitrogen, oxygen, sulfur and selenium atoms; preferably, the heterocycloalkyl is chosen from epoxide, piperazinyl, piperidyl, morpholinyl, dithiolanyl, preferably 1,2-dithiolanyl;
an “alkyl” radical is a linear or branched, in particular C₁-C₆ and preferably C₁-C₄ saturated hydrocarbon-based radical;
the expression “optionally substituted” attributed to the alkyl, alkoxy, alkylthio or (di)alkylamino radical means that the alkyl radical of each of the radicals may be substituted with one or more atoms or groups chosen from i) halogen such as C₁, Br, ii) hydroxyl, iii) C₁-C₄ alkoxy, iv) acylamino, v) (di)(C₁-C₄)(alkyl)amino; vi) (hetero)aryl such as phenyl and vii) (hetero)cycloalkyl;
an “alkoxy” radical is an alkyl-O— radical in which the alkyl part is as defined previously;
an “alkylthio” radical is an alkyl-S— radical in which the alkyl part is as defined previously;
a “(di)(C₁-C₆)(alkyl)amino” radical means an amino, (C₁-C₆)alkylamino or di(C₁-C₆)alkylamino radical;
an “alkenyl” radical is a linear or branched unsaturated hydrocarbon-based radical comprising one or more conjugated or non-conjugated double bonds;
an “alkynyl” radical is a linear or branched unsaturated hydrocarbon-based radical comprising one or more conjugated or non-conjugated triple bonds;
an “alkoxy” radical is an alkyl-oxy radical for which the alkyl radical is a linear or branched C₁-C₆ and preferentially C₁-C₄hydrocarbon-based radical;
the term “organic or mineral acid salt” more particularly means organic or mineral acid salts in particular chosen from a salt derived from i) hydrochloric acid HCl, ii) hydrobromic acid HBr, iii) sulfuric acid H₂SO₄, iv) alkylsulfonic acids: Alk-S(O)₂OH such as methanesulfonic acid and ethanesulfonic acid; v) arylsulfonic acids: Ar—S(O)₂OH such as benzenesulfonic acid and toluenesulfonic acid; vi) alkoxysulfinic acids: Alk-O—S(O)OH such as methoxysulfinic acid and ethoxysulfinic acid; vii) aryloxysulfinic acids such as tolueneoxysulfinic acid and phenoxysulfinic acid; viii) phosphoric acid H₃PO₄; ix) triflic acid CF₃SO₃H and x) tetrafluoroboric acid HBF₄; xi) organic carboxylic acids R^{o}—C(O)—OH (I′z), in which formula (I′z) R^{o} represents a (hetero)aryl group such as phenyl, (hetero)aryl(C₁-C₄)alkyl group such as benzyl, or (C₁-C₁₀)alkyl, said alkyl group being optionally substituted preferably with one or more hydroxyl groups or amino or carboxyl radicals, R^{o} preferably denoting a (C₁-C₆)alkyl group optionally substituted with 1, 2 or 3 hydroxyl or carboxyl groups; more preferentially, the monocarboxylic acids of formula (I′z) are chosen from acetic acid, glycolic acid, lactic acid, and mixtures thereof, and more particularly from acetic acid and lactic acid; and the polycarboxylic acids are chosen from tartaric acid, succinic acid, fumaric acid, citric acid and mixtures thereof; and xii) amino acids including more carboxylic acid radicals than amino groups, such as γ-carboxyglutamic acid, aspartic acid or glutamic acid, in particular γ-carboxyglutamic acid;
an “anionic counterion” is an anion or an anionic group associated with the cationic charge; more particularly, the anionic counterion is chosen from: i) halides such as chloride or bromide; ii) nitrates; iii) sulfonates, including C₁-C₆ alkylsulfonates: Alk-S(O)₂O⁻ such as methylsulfonate or mesylate and ethylsulfonate; iv) arylsulfonates: Ar—S(O)₂O⁻ such as benzenesulfonate and toluenesulfonate or tosylate; v) citrate; vi) succinate; vii) tartrate; viii) lactate; ix) alkyl sulfates: Alk-O—S(O)O— such as methyl sulfate and ethyl sulfate; x) aryl sulfates: Ar—O—S(O)O— such as benzene sulfate and toluene sulfate; xi) alkoxy sulfates: Alk-O—S(O)₂O⁻ such as methoxy sulfate and ethoxy sulfate; xii) aryloxy sulfates: Ar—O—S(O)₂O⁻; xiii) phosphate; xiv) acetate; xv) triflate; and xvi) borates such as tetrafluoroborate.
the “solvates” represent hydrates and also the combination with linear or branched C₁-C₄ alcohols such as ethanol, isopropanol or n-propanol.
the term “chromophore” means a radical derived from a colourless or coloured compound that is capable of absorbing in the UV and/or visible radiation range at a wavelength λ_{abs} of between 250 and 800 nm. Preferably, the chromophore is coloured, i.e. it absorbs wavelengths in the visible range, i.e. preferably between 400 and 800 nm. Preferably, the chromophores appear coloured to the eye, particularly between 400 and 700 nm (Ullmann's Encyclopedia, 2005, Wiley-VcH, Verlag “Dyes, General Survey”, § 2.1 Basic Principle of Color);
the term “fluorescent chromophore” means a chromophore which is also capable of re-emitting in the visible range at an emission wavelength λₑₘ of between 400 and 800 nm, and higher than the absorption wavelength, preferably with a Stoke's shift, i.e. the difference between the maximum absorption wavelength and the emission wavelength is at least 10 nm. Preferably, fluorescent chromophores are derived from fluorescent dyes that are capable of absorbing in the visible range λ_{abs}, i.e. at a wavelength of between 400 and 800 nm, and of re-emitting in the visible range at a λₑₘ between 400 and 800 nm. More preferentially, fluorescent chromophores are capable of absorbing at a λ_{abs} of between 420 and 550 nm and of re-emitting in the visible range at a λₑₘ between 470 and 600 nm;
the term “optical brightening chromophore” means a chromophore derived from an optical brightening compound or “optical brighteners, optical brightening agents (OBAs)” or “fluorescent brightening agents (FBAs)” or “fluorescent whitening agents (FWAs)”, i.e. agents which absorb UV radiation, i.e. at a wavelength λ_{abs} of between 250 and 350 nm, and of subsequently re-emitting this energy by fluorescence in the visible range at an emission wavelength λₑₘ of between 400 and 600 nm, i.e. wavelengths between blue-violet and blue-green with a maximum in the blue range. Optical brightening chromophores are thus colourless to the eye;
the term “UV-A screening agent” means a chromophore derived from a compound which screens out (or absorbs) UV-A ultraviolet rays at a wavelength of between 320 and 400 nm. A distinction may be made between short UV-A screening agents (which absorb rays at a wavelength of between 320 and 340 nm) and long UV-A screening agents (which absorb rays at a wavelength of between 340 and 400 nm);
the term “UV-B screening agent” means a chromophore derived from a compound which screens out (or absorbs) UV-B ultraviolet rays at a wavelength of between 280 and 320 nm.

Furthermore, unless otherwise indicated, the limits delimiting the extent of a range of values are included in that range of values.

### a) The PHA Copolymer(s)

The polyhydroxyalkanoate PHA copolymer of the invention contains several different repeating polymer units (A) as defined previously.

The term “copolymer” means that said polymer is derived from the polycondensation of different polymeric repeating units with each other; for example, when said polymer is derived from the polycondensation of polymeric repeating units (A), the units (A) are different from each other (more precisely, the groups R¹ are different from one repeating unit to another) and when said polymer is derived from the polycondensation of polymeric repeating units (A) with (B), the polymeric units (A) are different from the polymeric units (B) (more precisely, R¹ is different from R²).

Preferably, the PHA copolymer consists of a succession of the following units (A), and also the optical or geometrical isomers thereof, the organic or mineral acid or base salts thereof, and the solvates thereof such as hydrates:

—[—O—CH(R¹)—CH₂—C(O)—]— unit (A) with R₁ as defined previously.

According to a particular embodiment of the invention, a) is a PHA copolymer which contains or preferably consists of at least two different repeating polymer units chosen from units (A) and (B) as defined previously.

According to a particular embodiment of the invention, the PHA copolymer(s) consist of two different repeating polymer units chosen from the units (A) and (B) as defined previously.

Thus, a) is preferentially a polyhydroxyalkanoate (PHA) copolymer which contains, and preferably consists of, at least two different repeating polymer units chosen from the units (A) and (B) below, and also the optical or geometrical isomers thereof, the organic or mineral acid or base salts thereof, and the solvates thereof such as hydrates:

—[—O—CH(R¹)—CH₂—C(O)—]— unit (A)

—[—O—CH(R²)—CH₂—C(O)—]— unit (B)

in which polymer units (A) and (B):
R¹ represents a saturated or unsaturated, linear or branched, non-cyclic hydrocarbon-based chain, or a saturated or unsaturated non-aromatic cyclic hydrocarbon-based chain, comprising from 3 to 30 carbon atoms; preferably, the hydrocarbon-based chain is chosen from i) linear or branched (C₅-C₂₃)alkyl, ii) linear or branched (C₅-C₂₈)alkenyl, iii) linear or branched (C₅-C₂₈)alkynyl, iv) preferably, the hydrocarbon-based group is linear;
said hydrocarbon-based chain being:
   substituted with one or more groups chosen from:
      A) R^{3′}—C(X)—C(R⁴)(R⁵)—C(X′)—[Y]ₙ—* and B) R³—C(X)—C(—[Y]ₙ—*)(R⁴)—C(X)—R⁶ with:
R^{3′} and R⁶, which may be identical or different, representing a group chosen from optionally substituted (C₁-C₆)alkyl, optionally substituted (C₁-C₆)alkoxy, optionally substituted (C₁-C₆)alkylthio, optionally substituted (di)(C₁-C₆)(alkyl)amino, (hetero)aryl, (hetero)cycloalkyl, (hetero)aryloxy, (hetero)cycloalkyloxy, (hetero)arylthio, (hetero)cycloalkylthio, (hetero)arylamino, (hetero)cycloalkylamino; preferably representing a group chosen from (C₁-C₆)alkyl such as methyl, (C₁-C₆)alkoxy such as ethoxy;
R⁴ and R⁵, which may be identical or different, represent a hydrogen atom or a group chosen from (C₁-C₆)alkyl; preferably, R⁴ and R⁵ are identical, and particularly represent a hydrogen atom;
n is 0 or 1;
X and X′, which may be identical or different, represent an oxygen or sulfur atom or a group N—Rₐ with Rₐ representing a hydrogen atom or a (C₁-C₄)alkyl group; preferably, X and X are identical and particularly represent an oxygen atom;
Y represents a heteroatom chosen from O, S, N—Rₐ with Rₐ as defined previously; preferably, Y represents an oxygen atom;
* represents the point of attachment of group A or B) connected to the rest of the PHA copolymer(s); and
the radicals R¹ also possibly being:
   substituted with one or more atoms or groups chosen from: a) halogen such as chlorine or bromine, b) hydroxyl, c) thiol, d) (di)(C₁-C₄)(alkyl)amino, e) (thio)carboxy, f) (thio)carboxamide —C(O)—N(Rₐ)₂ or C(S)—N(Rₐ)₂, g) cyano, h) iso(thio)cyanate, i) (hetero)aryl such as phenyl or furyl, and j) (hetero)cycloalkyl, k) cosmetic active agent; I) R—X with R representing a group chosen from α) cycloalkyl such as cyclohexyl, β) heterocycloalkyl such as sugar, preferably monosaccharide such as glucose, γ) (hetero)aryl such as phenyl, m) thiosulfate; X representing a′) O, S, N(R′ₐ) or Si(R′_{b})(R′_{c}), b′) S(O)ᵣ, or (thio)carbonyl, c′) or combinations of a′) with b′) such as (thio)ester, (thio)amide, (thio)urea or sulfonamide; R′ₐ representing a hydrogen atom, or a (C₁-C₄)alkyl group or an aryl(C₁-C₄)alkyl group such as benzyl; preferably, Rₐ represents a hydrogen atom; R′_{b} and R′_{c}, which may be identical or different, represent a (C₁-C₄)alkyl or (C₁-C₄)alkoxy group, particularly only one substituent; preferably chosen from b) halogen, and j) such as epoxide; and/or
   optionally interrupted with one or more a′) heteroatoms such as O, S, N(Rₐ) and Si(R_{b})(R_{c}), b′) S(O)ᵣ, (thio)carbonyl, c′) or combinations of a′) with b′) such as (thio)ester, (thio)amide, (thio)urea, sulfonamide, preferably ester —O—C(O)— or —C(O)—O— with r being equal to 1 or 2, Rₐ being as defined previously; preferably, Rₐ represents a hydrogen atom, R_{b} and R_{c} being as defined previously; and
R² represents a hydrocarbon-based chain as defined previously for R¹; preferably the hydrocarbon-based chain of the radical R² has a carbon number corresponding to the number of carbon atoms in the radical R¹ minus at least one carbon atom, preferably corresponding to the number of carbon atoms in the radical R¹ minus two carbon atoms;
it being understood that (A) is different from (B) (i.e. R¹, and is different from R²).

According to a particular embodiment of the invention, the PHA is such that the radical R¹ represents a hydrocarbon-based chain chosen from i) (C₅-C₂₂)alkyl, linear or branched, preferably linear, ii) (C₅-C₂₂)alkenyl, linear or branched, preferably linear, said hydrocarbon-based chain being substituted, preferably at the end of the chain, with one or more groups (preferably a single group) chosen from A) R³—C(X)—C(R⁴)(R⁵)—C(X′)—[Y]ₙ—* with R³, R⁴, R⁵, X, X′, Y, n and * as defined previously; more preferentially, A) represents:

R³—C(O)—C(R⁴)(R⁵)—C(O)—[O]ₙ—* with R³, R⁴ and R⁵, n and * as defined previously. In particular, R⁴ and R⁵ represent a hydrogen atom. According to a preferred embodiment, n is 1.

According to another particular embodiment of the invention, the PHA is such that the radical R¹ represents a hydrocarbon-based chain chosen from i) linear or branched, preferably linear, (C₅-C₂₂)alkyl, ii) linear or branched, preferably linear, (C₅-C₂₂)alkenyl, said hydrocarbon-based chain being substituted, preferably at the end of the chain, with one or more groups (preferably a single group) chosen from) R³—C(X)—C(—[Y]ₙ—*)(R⁴)—C(X)—R⁶ with R³, R⁴, R⁶, X, X′, Y, n and * as defined previously; more preferentially, B) represents:

R³—C(O)—C(—[O]ₙ—*)(R⁴)—C(O)—R⁶ with R³, R⁴ and R⁵, n and * are as defined previously. In particular, R⁴ represents a hydrogen atom. Preferably, n is 0.

According to a particular embodiment of the invention, the PHA is such that the radical R¹ represents a hydrocarbon-based chain chosen from i) linear or branched, preferably linear, (C₇-C₂₂)alkyl, ii) linear or branched, preferably linear, (C₇-C₂₂)alkenyl, said hydrocarbon-based chain being:
substituted, preferably at the end of the chain, with a group chosen from A) and B) as defined previously; and
   R³ and R⁶, which may be identical or different, representing a group chosen from (C₁-C₆)alkyl such as methyl, (C₁-C₆)alkoxy such as ethoxy;
   R⁴ and R⁵, which may be identical or different, represent a hydrogen atom or a group chosen from (C₁-C₆)alkyl; preferably, R⁴ and R⁵ are identical, and particularly represent a hydrogen atom;
   n is 0 or 1;
   X and X′ are identical and particularly represent an oxygen atom;
   Y represents an oxygen atom;
   represents the point of attachment of the group A or B connected to the rest of the PHA copolymer(s); and
optionally substituted with one or more OH groups; and/or
optionally interrupted with one or more a′) heteroatoms chosen from O and S, b′) carbonyl, c′) and combinations of a′) with b′) such as ester; preferably interrupted with one or more O or S, more preferentially S.

According to a particular embodiment of the invention, the PHA is such that the radical R¹ represents a linear hydrocarbon-based chain chosen from i) (C₇-C₁₅)alkyl, ii) (C₇-C₁₅)alkenyl, said hydrocarbon-based chain being substituted with A) or B) as defined previously.

According to another embodiment, the hydrocarbon-based chain of the radical R¹ of the invention is 1) either substituted with A) or B) as defined previously, and uninterrupted, 2) or substituted with A) or B) as defined previously and interrupted.

According to a particular embodiment of the invention, the radical R¹ is of the following formula —(CH₂)ᵣ—X-(ALK)ᵤ-G with X being as defined previously, in particular representing O, S, N(Rₐ), preferably S, ALK represents a linear or branched (C₁-C₁₀)alkylene chain, linear or branched (C₂-C₁₀)alkenylene, preferably a linear (C₁-C₁₀)alkylene chain, more particularly (C₁-C₈)alkylene, it being possible for ALK to be substituted with one or more hydroxyl groups and/or optionally with one or more ester groups —C(O)—O— or —O—C(O)—, r represents an integer between 6 and 11 inclusive, preferably between 7 and 10 such as 8; u is equal to 0 or 1; and G represents a radical A) or B) as defined previously.

According to a particular embodiment of the invention, the group R² represents a hydrocarbon-based chain chosen from linear or branched (C₃-C₂₃)alkyl and linear or branched (C₃-C₂₃)alkenyl, in particular a linear hydrocarbon-based group, more particularly (C₄-C₂₀)alkyl or (C₄-C₂₀)alkenyl, said hydrocarbon-based chain being optionally substituted notably with A) or B) as defined previously. Preferably, R² represents a hydrocarbon-based group having a number of carbon atoms corresponding to the number of carbon atoms in the radical R¹ minus at least one carbon atom, preferably corresponding to the number of carbon atoms in the radical R¹ minus two carbon atoms.

More particularly, the PHA copolymer(s) according to the invention comprise the repeating unit of formula (I), and also the optical or geometrical isomers thereof, the organic or mineral acid or base salts thereof, and the solvates thereof such as hydrates:

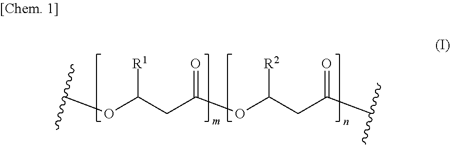

in which formula (I):
R¹ and R² are as defined previously;
m and n are integers greater than or equal to 1; preferably, the sum n+m is inclusively between 450 and 1400;
preferably, m<n with R¹ representing an alkyl group substituted with A) or B) and optionally interrupted, an alkenyl group substituted with A) or 1) and optionally interrupted or an alkynyl group substituted with A) or 5) and optionally interrupted, and R² represents an alkyl group.

In particular, the stereochemistry of the carbon atoms bearing the radicals R¹ and R² is of the same (R) or (S) configuration, preferably of (R) configuration.

According to a particular embodiment, the PHA copolymer(s) of composition a) contain three different repeating polymer units (A), (B) and (C), and preferably consist of three different polymer units (A), (B) and (C) below, and also the optical or geometrical isomers thereof and the solvates thereof such as hydrates:

—[—O—CH(R¹)—CH₂—C(O)—]— unit (A)

—[—O—CH(R²)—CH₂—C(O)—]— unit (B)

—[—O—CH(R³)—CH₂—C(O)—]— unit (C)

in which polymer units (A), (B) and (C):
R¹ and R² are as defined previously;
R³ represents a saturated or unsaturated, cyclic or non-cyclic, linear or branched hydrocarbon-based chain comprising from 1 to 30 carbon atoms; said chain being optionally substituted with one or more groups notably chosen from A) or B) as defined previously, optionally substituted with the groups a) to j) as defined for R¹ and/or optionally interrupted with one or more heteroatoms or groups a′) to c′) as defined for R¹; in particular, said chain represents a hydrocarbon-based group chosen from linear or branched (C₁-C₂₃)alkyl, and linear or branched (C₂-C₂₃)alkenyl, in particular a linear hydrocarbon-based group, more particularly (C₄-C₂₀)alkenyl; preferably, the hydrocarbon-based group has a number of carbon atoms corresponding to the number of carbon atoms in the radical R¹, or else corresponding to the number of carbon atoms in the radical R¹ minus at least three carbon atoms, preferably corresponding to the number of carbon atoms in the radical R¹ minus four carbon atoms; and
   it being understood that:
(A) is different from (B) and (C), (B) is different from (A) and (C), and (C) is different from (A) and (B); and
preferably, the molar percentage of unit (A) is lower than the molar percentage of unit (B) and the molar percentage of unit (C) notably if R² represents an alkyl group and/or R³ represents an alkyl group.

According to a particular embodiment of the invention, the PHA copolymer(s) comprise the repeating unit of formula (II), and also the optical or geometrical isomers thereof, the organic or mineral acid or base salts thereof, and the solvates thereof such as hydrates:

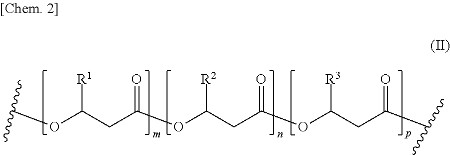

in which formula (II):
R¹, R² and R³ are as defined previously;
m, n and p are integers greater than or equal to 1; preferably, the sum n+m+p is inclusively between 450 and 1400; and
preferably, m<n+p when R¹ represents an alkyl group substituted with A) or B) as defined previously, optionally substituted with one or more groups chosen from a) to m) as defined for R¹ and optionally interrupted with one or more heteroatoms or groups a′) to c′), alkenyl substituted with A) or 5) as defined previously, optionally substituted with one or more groups chosen from a) to m) as defined for R¹ and optionally interrupted with one or more heteroatoms or groups a′) to c′) or alkynyl substituted with A) or 5) as defined previously, optionally substituted with one or more groups chosen from a) to m) as defined for R¹ and optionally interrupted with one or more heteroatoms or groups a′) to c′), and R² and R³ represent an alkyl group.

According to a particular embodiment, the PHA copolymer(s) of composition a) contain four different repeating polymer units (A), (B), (C) and (D), and preferably consist of four different polymer units (A), (B), (C) and (D), below, and also the optical or geometrical isomers thereof, the organic or mineral acid or base salts thereof, and the solvates thereof such as hydrates:

—[—O—CH(R¹)—CH₂—C(O)—]— unit (A)

—[—O—CH(R²)—CH₂—C(O)—]— unit (B)

—[—O—CH(R³)—CH₂—C(O)—]— unit (C)

—[—O—CH(R⁴)—CH₂—C(O)—]— unit (D)

in which polymer units (A), (B), (C) and (D):
R¹, R² and R³ are as defined previously;
R⁴ is as defined for R², and in particular represents a hydrocarbon-based chain chosen from linear or branched (C₄-C₂₃)alkyl, optionally substituted with one or more groups notably chosen from A) or B) as defined previously, optionally substituted with the groups a) to m) as defined for R¹ and/or optionally interrupted with one or more heteroatoms or groups a′) to c′) as defined for R¹ a) to m); and it being understood that:
(A) is different from (B), (C) and (D), (B) is different from (A), (C) and (D), (C) is different from (A), (B) and (D), and (D) is different from (A), (B) and (C).

Preferably, R² represents a linear or branched (C₆-C₃₀)alkyl group and R⁴ represents an alkyl group comprising at least one carbon atom less than R², preferably at least two carbon atoms less than R², in the (C₄-C₂₈) range.

According to a particular embodiment of the invention, R³ includes a radical identical to R¹ except that it includes at least one carbon atom less, preferably two carbon atoms less, than R².

According to a particular embodiment of the invention, the PHA copolymer(s) comprise the repeating unit of formula (III), and also the optical or geometrical isomers thereof, the organic or mineral acid or base salts thereof, and the solvates thereof such as hydrates:

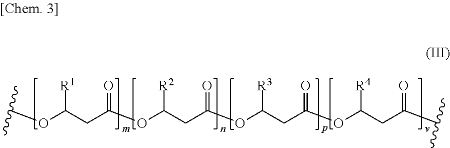

in which formula (III):
R¹, R², R³ and R⁴ are as defined previously;
m, n, p and v are integers greater than or equal to 1;
preferably, the sum n+m+p+v is inclusively between 450 and 1400; and
preferably, R² and R⁴ represent an alkyl group, and R³ represents an alkyl group optionally substituted with one or more groups notably chosen from A) or B) as defined previously, optionally substituted with the groups a) to m) as defined for R¹ and/or optionally interrupted with one or more heteroatoms or groups a′) to c′) as defined for R¹, alkenyl optionally substituted with one or more groups notably chosen from A) or B) as defined previously, optionally substituted with the groups a) to m) as defined for R¹ and/or optionally interrupted with one or more heteroatoms or groups a′) to c′) as defined for R¹ or alkynyl optionally substituted with one or more groups notably chosen from A) or B) as defined previously, optionally substituted with the groups a) to m) as defined for R¹ and/or optionally interrupted with one or more heteroatoms or groups a′) to c′) as defined for R¹, and/or interrupted with a′) to c′) as defined previously for R¹, then n>m+v; more preferentially, n+p>m+v.

According to one embodiment, the PHA copolymer(s) of composition a) more particularly contain five different repeating polymer units (A), (B), (C), (D) and (E), and preferably consist of five different polymer units (A), (B), (C), (D) and (E), below, and also the optical or geometrical isomers thereof, the organic or mineral acid or base salts thereof, and also the solvates thereof such as hydrates:

—[—O—CH(R¹)—CH₂—C(O)—]— unit (A)

—[—O—CH(R²)—CH₂—C(O)—]— unit (B)

—[—O—CH(R³)—CH₂—C(O)—]— unit (C)

—[—O—CH(R⁴)—CH₂—C(O)—]— unit (D)

—[—O—CH(R⁵)—CH₂—C(O)—]— unit (E)

in which polymer units (A), (B), (C), (D) and (E):
R¹, R², R³ and R⁴ are as defined previously; and
R⁵ represents a saturated, linear or branched, cyclic or non-cyclic hydrocarbon-based chain comprising from 3 to 30 carbon atoms optionally substituted with one or more groups notably chosen from A) or B) as defined previously, optionally substituted with the groups a) to m) as defined for R¹ and/or optionally interrupted with one or more heteroatoms or groups a′) to c′) as defined for R¹ a) to m), and in particular represents a hydrocarbon-based chain chosen from linear or branched (C₄-C₂₃)alkyl, optionally substituted with one or more groups notably chosen from A) or B) as defined previously, optionally substituted with the groups a) to m) as defined for R¹ and/or optionally interrupted with one or more heteroatoms or groups a′) to c′) as defined for R¹ a) to m); preferably, the hydrocarbon-based chain has a carbon number corresponding to the number of carbon atoms in the radical R³ minus at least one carbon atom, preferably corresponding to the number of carbon atoms in the radical R³ minus at least two carbon atoms, preferably minus two carbon atoms: it being understood that:
(A) is different from (B), (C), (D) and (E); (B) is different from (A), (C), (D) and (E); (C) is different from (A), (B), (D) and (E); (D) is different from (A), (B), (C) and (E); and (E) is different from (A), (B), (C) and (D).

According to a particular embodiment of the invention, the PHA copolymer(s) comprise the repeating unit of formula (IV), and also the optical or geometrical isomers thereof, the organic or mineral acid or base salts thereof, and the solvates thereof such as hydrates:

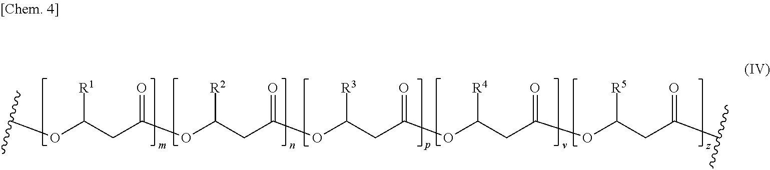

in which formula (IV):
R¹, R², R³, R⁴ and R⁵ are as defined previously;
m, n, p, v and z are integers greater than or equal to 1; preferably, the sum n+m+p+v+z is inclusively between 450 and 1400;
preferably, when R¹, R², R³, R⁴ and R⁵ represent an unsubstituted and uninterrupted alkyl group, then m>n+p+v+z; and
preferably, when R² and R⁴ represent an alkyl group and the groups R³ and R⁵ represent an alkyl group optionally substituted with one or more groups notably chosen from A) or B) as defined previously, optionally substituted with the groups a) to m) as defined for R¹ and/or optionally interrupted with one or more heteroatoms or groups a′) to c′) as defined for R¹; alkenyl optionally substituted with one or more groups notably chosen from A) or B) as defined previously, optionally substituted with the groups a) to m) as defined for R¹ and/or optionally interrupted with one or more heteroatoms or groups a′) to c′) as defined for R¹; or alkynyl optionally substituted with one or more groups notably chosen from A) or B) as defined previously, optionally substituted with the groups a) to m) as defined for R¹ and/or optionally interrupted with one or more heteroatoms or groups a′) to c′) as defined for R¹, then n>m+v+z; more preferentially, n+p>m+v+z.

According to a particular embodiment of the invention, in the PHA copolymer(s) according to the invention, the unit (A) comprises a hydrocarbon-based chain as defined previously, in particular i), said unit (A) preferably being present in a molar percentage ranging from 0.1% to 99%, more preferentially a molar percentage ranging from 0.5% to 50%, even more preferentially a molar percentage ranging from 1% to 40%, better still a molar percentage ranging from 2% to 30%, or a molar percentage ranging from 5% to 20%.

According to a more particular embodiment of the invention in the PHA copolymer(s) according to the invention, the unit (A) is preferably present in a molar percentage ranging from 0.5% to 99%, more preferentially a molar percentage ranging from 1% to 50%, even more preferentially a molar percentage ranging from 5% to 40%, better still a molar percentage ranging from 10% to 30%; the unit (B) is present in a molar percentage ranging from 2% to 40%; and the unit (C) is present in a molar percentage ranging from 0.5% to 20% relative to the sum of the units (A), (B) and (C). Advantageously, the PHA copolymer(s) of the invention comprise from 2 mol % to 10 mol % of units (B), and from 0.5 mol % to 7 mol % of units (C); more advantageously, the copolymer comprises from 5 mol % to 35 mol % of units (B), and from 0.5 mol % to 7 mol % of units (C).

According to a more particular embodiment of the invention, the PHA copolymer(s) according to the invention are such that, in the PHA copolymer(s) a):
the unit (A) comprises a hydrocarbon-based chain as defined previously, said unit (A) being present in a molar percentage ranging from 0.1% to 99%, preferably a molar percentage ranging from 0.5% to 50%, more preferentially a molar percentage ranging from 1% to 40%, even more preferentially a molar percentage ranging from 2% to 30%, better still a molar percentage ranging from 5% to 20%, even better still a molar percentage ranging from 10% to 30% of units (A); and
the unit (B) is present in a molar percentage ranging from 1% to 40%, preferentially a molar percentage from 2% to 10%, more preferentially a molar percentage from 5% to 35% of units (B); and/or
the unit (C) is present in a molar percentage ranging from 0.5% to 20%, preferentially a molar percentage from 1% to 7%, more preferentially from 0.5 mol % to 7 mol % of units (C).

Preferably, when R¹ of the unit (A) is a saturated hydrocarbon-based chain, said unit (A) is present in a molar percentage of greater than 30%, more particularly greater than 50%, more preferentially greater than 60%, preferably between 60% and 90%.

The values of the molar percentages of the units (A), (B) and (C) of the PHA copolymer(s) are calculated relative to the total number of moles of (A)+(B) if the copolymer(s) do not comprise any additional units (C); otherwise, if the copolymer(s) of the invention contain three different units (A), (B) and (C), then the molar percentage is calculated relative to the total number of moles (A)+(B)+(C); otherwise, if the copolymer(s) of the invention contain four different units (A), (B), (C) and (D), then the molar percentage is calculated relative to the total number of moles (A)+(B)+(C)+(D); otherwise, if the copolymer(s) of the invention contain five different units (A), (B), (C), (D) and (E), then the molar percentage is calculated relative to the total number of moles (A)+(B)+(C)+(D)+(E).

According to a particular embodiment, R¹ represents a group chosen from:
i) *-(ALK₁)—O—C(O)—CH₂—C(O)—R^{3′};
ii) *—(CH₂)ₚ—S-ALK—O—C(O)—CH₂—C(O)—R^{3′};
iii) *—(CH₂)ₚ—CH═CH-ALK₁—O—C(O)—CH₂—C(O)—R³;
iv) *—(CH₂)ₚ—O-ALK₁—C(O)—CH₂—C(O)—R^{3′};
v) *—(CH₂)ₚ—CH(OH)—CH[C(O)—CH₃]—C(O)—R^{3′};
iv) *—(CH₂)ₚ—CH[C(O)—CH₃]—C(O)—R^{3′}; and
vi) *—(CH₂)ₚ—O—C(O)-ALK₁—C(O)—CH₂—C(O)—R^{3′}
vii) *—(CH₂)ₚ—S-ALK₃-[O—C(O)—CH₂—C(O)—R³]₂
   where p is an integer between 3 and 15, ALK₁ denotes a divalent linear or branched C₁-C₁₅ hydrocarbon-based radical optionally substituted with a hydroxyl group, preferably an alkylene group optionally substituted with a hydroxyl group, ALK₃ denotes a trivalent linear or branched C₁-C₁₅ hydrocarbon-based radical, optionally substituted with a hydroxyl group, preferably ALK₃ represents a trivalent linear or branched (C₂-C₆)alkylene group, more preferably a trivalent linear or branched (C₃-C₅)alkylene group such as trivalent propylene optionally substituted with a hydroxyl group; and R^{3′} is as defined previously, preferably a (C₁-C₄)alkyl group such as methyl or (C₁-C₄)alkoxy such as methoxy or ethoxy; preferably R¹ is selected from i) to vi).

Preferably, R¹ represents —(CH₂)ₚ—S-ALK₁—O—C(O)—CH₂—C(O)—CH₃ where p is an integer between 3 and 15, ALK₁ denotes a divalent linear or branched C₁-C₁₅ hydrocarbon-based radical optionally substituted with one or more hydroxyl groups, preferably linear, particularly *—(CH₂)₈—S—(CH₂)₃—O—C(O)—CH₂—C(O)—CH₃.

According to another embodiment of the invention R¹ represents *—(CH₂)ₚ—S-ALK₃-[O—C(O)—CH₂—C(O)—R³]₂ preferably *—(CH₂)ₚ—S-ALK₃—[O—C(O)—CH₂—C(O)—CH₃]₂ where p is an integer between 3 and 15 such as 8, and ALK₃ represents a trivalent linear or branched (C₂-C₆)alkylene group, more preferably a trivalent linear or branched (C₃-C₅)alkylene group such as trivalent propylene.

Preferentially, the PHA copolymer(s) of the invention comprise the following repeating units, and also the optical or geometrical isomers thereof, the organic or mineral acid or base salts thereof, and the solvates thereof such as hydrates:

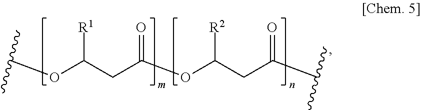

| Compound | R¹ | R² |
|---|---|---|
| (1) | *-(ALK₁)-O—C(O)—CH₂—C(O)—CH₃ | -ALK₂ |
| (2) | *—(CH₂)ₚ—S-ALK₁-O—C(O)—CH₂—C(O)—CH₃ | -ALK₂ |
| (3) | *—(CH₂)ₚ—CH═CH-ALK₁-O—C(O)—CH₂—C(O)—CH₃ | -ALK₂ |
| (5) | *—(CH₂)ₚ—CH(OH)—CH[C(O)—CH₃]—C(O)R^{3′} | -ALK₂ |
| (6) | *—(CH₂)ₚ—CH[C(O)—CH₃]—C(O)—R^{3′} | -ALK₂ |
| (7) | *—(CH₂)ₚ—S-ALK₁-O—C(O)—CH₂—C(O)—CH₃ | -ALK₂ |
| (8) | *—(CH₂)ₚ—CH═CH-ALK₁-O—C(O)—CH₂—C(O)—CH₃ | - ALK₂ |
| (9) | *-(ALK₁)-O—C(O)—CH₂—C(O)—CH₃ | - ALK₂ |
| (10) | *—(CH₂)ₚ—S-ALK₁-O—C(O)—CH₂—C(O)—CH₃ | - ALK₂ |
| (11) | *—(CH₂)ₚ—S-ALK₃-[O—C(O)—CH₂—C(O)—CH₃]₂ | - ALK₂ |

where p is an integer between 3 and 15, ALK₁ denotes a divalent linear or branched C₁-C₁₅ hydrocarbon-based radical optionally substituted with a hydroxyl group, preferably an alkylene group optionally substituted with a hydroxyl group, and R₃, is as defined previously, preferably a (C₁-C₄)alkoxy group such as methoxy or ethoxy, ALK₂ denotes a C₃-C₂₀ alkyl radical; and ALK₃ denotes a trivalent linear or branched C₁-C₁₅ hydrocarbon-based radical, optionally substituted with a hydroxyl group, preferably ALK₃ represents a trivalent linear or branched (C₂-C₆)alkylene group, more preferably a trivalent linear or branched (C₃-C₅)alkylene group such as trivalent propylene optionally substituted with a hydroxyl group, and R^{3′} is as defined previously, preferably a (C₂-C₆)alkoxy or (C₂-C₆)alkyl group such as CH₃.

In particular, the stereochemistry of the carbon atoms bearing the radicals R¹ and R² is of the same (R) or (S) configuration, preferably of (R) configuration.

More preferentially, the PHA copolymer(s) have the following formulae, and also the optical isomers thereof, the organic or mineral acid or base salts thereof, and the solvates thereof such as hydrates:

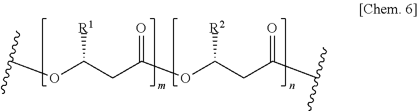

| Compound | R¹ | R² |
|---|---|---|
| (1′) | *-(ALK₁)-O—C(O)—CH₂—C(O)—CH₃ | -ALK₂ |
| (2′) | *—(CH₂)ₚ—S-ALK₁-O—C(O)—CH₂—C(O)—CH₃ | -ALK₂ |
| (3′) | *—(CH₂)ₚ—CH═CH-ALK₁-O—C(O)—CH₂—C(O)—CH₃ | -ALK₂ |
| (5′) | *—(CH₂)ₚ—CH(OH)—CH[C(O)—CH₃]—C(O)R^{3′} | -ALK₂ |
| (6′) | *—(CH₂)ₚ—CH[C(O)—CH₃]—C(O)—R^{3′} | -ALK₂ |
| (7′) | *—(CH₂)ₚ—S-ALK₁-O—C(O)—CH₂—C(O)—CH₃ | -ALK₂ |
| (8′) | *—(CH₂)ₚ—CH═CH-ALK₁-O—C(O)—CH₂—C(O)—CH₃ | - ALK₂ |
| (9′) | *-(ALK₁)-O—C(O)—CH₂—C(O)—CH₃ | - ALK₂ |
| (10′) | *—(CH₂)ₚ—S-ALK₁-O—C(O)—CH₂—C(O)—CH₃ | - ALK₂ |
| (11′) | *—(CH₂)ₚ—S-ALK₃-[O—C(O)—CH₂—C(O)—CH₃]₂ | - ALK₂ |

More preferentially, the PHA copolymer(s) have the following formulae, and also the optical isomers thereof, the organic or mineral acid or base salts thereof, and the solvates thereof such as hydrates:

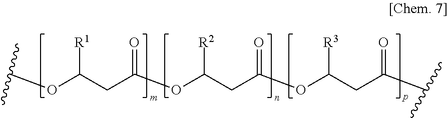

| Compound | R¹ | R² | R³ |
|---|---|---|---|
| (1″) | *—(CH₂)₆—S—(CH₂)₃—O—C(O)— | *—(CH₂)₆—H | *—(CH₂) [symbol] —S—(CH₂)₂—O—C(O)— |
| | CH₂—C(O)—CH₃ | | CH₂—C(O)—CH₃ |
| (2″) | *—(CH₂)ₚ—S—(CH₂)₂—O—C(O)— | *(CH₂)₍ₚ₋₂₎—H | *—(CH₂)₍ₚ₋₂₎—S—(CH₂)₂—O—C(O)— |
| | CH₂—C(O)—CH₃ | | CH₂—C(O)—CH₃ |
| (3″) | *—(CH₂)ₚ—CH═CH—(CH₂—O— | *(CH₂)ₚ₋₂—H | *—(CH₂)₍ₚ₋₂₎—S—(CH₂)₂—O—C(O)— |
| | C(O)—CH₂—C(O)—CH₃ | | CH₂—C(O)—CH₃ |
| (4″) | *—(CH₂)ₚ—CH₂—CH₂—O—C(O)— | *(CH₂)ₚ₋₂—H | *—(CH₂)₍ₚ₋₂₎—S—(CH₂)₂—O—C(O)— |
| | CH₂—C(O)—CH₃ | | CH₂—C(O)—CH₃ |
| (5″) | *—(CH₂)ₚ—CH₂—CH₂—O—CH₂— | *(CH₂)ₚ₋₂—H | *—(CH₂)₍ₚ₋₂₎—S—(CH₂)₂—O—C(O)— |
| | C(O)—CH₂—C(O)—O—CH₂—CH₃ | | CH₂—C(O)—CH₃ |
| (6″) | *—(CH₂)ₚ—CH(OH)—CH₂—O— | *(CH₂)ₚ₋₂—H | *—(CH₂)₍ₚ₋₂₎—S—(CH₂)₂—O—C(O)— |
| | C(O)—CH₂—CH₂—C(O)—CH₂— | | CH₂—C(O)—CH₃ |
| | C(O)—CH₃ | | |
| (7″) | *—(CH₂)ₚ—CH(OH)—CH[C(O)— | *(CH₂)ₚ₋₂—H | *—(CH₂)₍ₚ₋₂₎—S—(CH₂)₂—O—C(O)— |
| | CH₃]—C(O)-R | | CH₂—C(O)—CH₃ |
| (8″) | *—(CH₂)ₚ—S-ALK₃—[O—C(O)— | *(CH₂)ₚ₋₂—H | *—(CH₂)ₚ₋₂—S-ALK₃—[O—C(O)— |
| | CH₂—C(O)—CH₃]₂ | | CH₂—C(O)—CH₃]₂ |

[symbol]

indicates data missing or illegible when filed

with p as defined previously.

More particularly, the stereochemistry of the carbon atoms bearing the radicals R¹, R² and R³ is of the same (R) or (S) configuration, preferably of (R) configuration.

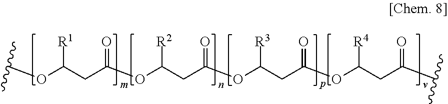

| Compound | R¹ | R² | R³ | R⁴ |
|---|---|---|---|---|
| (1″) | *—(CH₂)₆—S—(CH₂)₃—O— | *-n-hexyle | *—(CH₂)₆—S—(CH₂)₃—O— | *-n-butyle |
| | C(O)—CH₂—C(O)—CH₃ | | C(O)—CH₂—C(O)—CH₃ | |
| (2″) | *—(CH₂)ₚ—S—(CH₂)₃—O— | *(CH₂)₍ₚ₋₂₎—H | *—(CH₂)₍ₚ₋₂₎—S—(CH₂)₂— | *(CH₂)₍ₚ₋₄₎—H |
| | C(O)—CH₂—C(O)—CH₃ | | O—C(O)—CH₂—C(O)— | |
| | | | CH₃ | |
| (3″) | *—(CH₂)ₚ—CH═CH—CH₂— | *(CH₂)₍ₚ₋₂₎—H | *—(CH₂)₍ₚ₋₂₎—S—(CH₂)₂— | *(CH₂)₍ₚ₋₄₎—H |
| | O—C(O)—CH₂—C(O)—CH₃ | | O—C(O)—CH₂—C(O)— | |
| | | | CH₃ | |
| (4″) | *—(CH₂)ₚ—CH₂—CH₂—O | *(CH₂)₍ₚ₋₂₎—H | *—(CH₂)₍ₚ₋₂₎—S—(CH₂)₂— | *(CH₂)₍ₚ₋₄₎—H |
| | C(O)—CH₂—C(O)—CH₃ | | O—C(O)—CH₂—C(O)— | |
| | | | CH₃ | |
| (5″) | *—(CH₂)ₚ—CH₂—CH₂—O | *(CH₂)₍ₚ₋₂₎—H | *—(CH₂)₍ₚ₋₂₎—S—(CH₂)₂— | *(CH₂)₍ₚ₋₄₎—H |
| | CH₂—C(O)—CH₂—C(O)—O | | O—C(O)—CH₂—C(O)— | |
| | CH₂—CH₃ | | CH₃ | |
| (6″) | *—(CH₂)ₚ—CH(OH)—CH₂— | *(CH₂)₍ₚ₋₂₎—H | *—(CH₂)₍ₚ₋₂₎—S—(CH₂)₂— | *(CH₂)₍ₚ₋₄₎—H |
| | O—C(O)—CH₂—CH₂—C(O)— | | O—C(O)—CH₂—C(O)— | |
| | CH₂—C(O)—CH₃ | | CH₃ | |
| (7″) | *—(CH₂)ₚ—CH(OH)— | *(CH₂)₍ₚ₋₂₎—H | *—(CH₂)₍ₚ₋₂₎—S—(CH₂)₂— | *(CH₂)₍ₚ₋₄₎—H |
| | CH[C(O)—CH₃]—C(O)-R | | O—C(O)—CH₂—C(O)— | |
| | | | CH₃ | |
| (8″) | *—(CH₂)ₚ—S-ALK₃—[O—C(O)— | *(CH₂)₍ₚ₋₂₎—H | *—(CH₂)₍ₚ₋₂₎—S—(CH₂)₂— | *(CH₂)₍ₚ₋₄₎—H |
| | CH₂—C(O)—CH₃]₂ | | O—C(O)—CH₂—C(O)— | |
| | | | CH₃ | |

More particularly, the stereochemistry of the carbon atoms bearing the radicals R¹, R², R³ and R⁴ is of the same (R) or (S) configuration, preferably of (R) configuration.

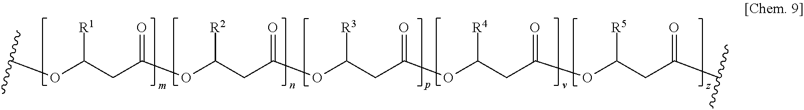

| | R¹ | R² | R³ | R⁴ | R⁵ |
|---|---|---|---|---|---|
| (1″) | *—(CH₂)₈—S— | *—(CH₂)₆—H | *—(CH₂)₆—S— | *-n-butyl | *—(CH₂)₄—S— |
| | (CH₂)₃—O— | | (CH₂)₃—O—C(O)— | | (CH₂)₃—O—C(O)— |
| | C(O)—CH₂— | | CH₂—C(O)—CH₃ | | CH₂—C(O)—CH₃ |
| | C(O)—CH₃ | | | | |
| (2″) | *—(CH₂)ₚ—S— | *(CH₂)₍ₚ₋₂₎— | *—(CH₂)₍ₚ₋₂₎—S— | *(CH₂)₍ₚ₋₄₎— | *—(CH₂)₍ₚ₋₄₎—S— |
| | (CH₂)₂—O— | H | (CH₂)₂—O—C(O)— | H | (CH₂)₂—O—C(O)— |
| | C(O)—CH₂— | | CH₂—C(O)—CH₃ | | CH₂—C(O)—CH₃ |
| | C(O)—CH₃ | | | | |
| (3″) | *—(CH₂)ₚ— | *(CH₂)₍ₚ₋₂₎— | *—(CH₂)₍ₚ₋₂₎—S— | *-n-(alkyl- | *—(CH₂)₍ₚ₋₄₎—S— |
| | CH═CH— | H | (CH₂)₂—O—C(O)— | 2) | (CH₂)₂—O—C(O)— |
| | CH₂—O— | | CH₂—C(O)—CH₃ | | CH₂—C(O)—CH₃ |
| | C(O)—CH₂— | | | | |
| | C(O)—CH₃ | | | | |
| (4″) | *—(CH₂)ₚ— | *(CH₂)₍ₚ₋₂₎— | *—(CH₂)₍ₚ₋₂₎—S— | *-n-(alkyl- | *—(CH₂)₍ₚ₋₄₎—S— |
| | CH₂—CH₂—O— | H | (CH₂)₂—O—C(O)— | 2) | (CH₂)₂—O—C(O)— |
| | C(O)—CH₂— | | CH₂—C(O)—CH₃ | | CH₂—C(O)—CH₃ |
| | C(O)—CH₃ | | | | |
| (5″) | *—(CH₂)ₚ— | *(CH₂)₍ₚ₋₂₎— | *—(CH₂)₍ₚ₋₂₎—S— | *-n-(alkyl- | *—(CH₂)₍ₚ₋₄₎—S— |
| | CH₂—CH₂—O— | H | (CH₂)₂—O—C(O)— | 2) | (CH₂)₂—O—C(O)— |
| | CH₂—C(O)— | | CH₂—C(O)—CH₃ | | CH₂—C(O)—CH₃ |
| | CH₂—C(O)— | | | | |
| | O—CH₂—CH₃ | | | | |
| (6″) | *—(CH₂)ₚ— | *(CH₂)₍ₚ₋₂₎— | *—(CH₂)₍ₚ₋₂₎—S— | *-n-(alkyl- | *—(CH₂)₍ₚ₋₄₎—S— |
| | CH(OH)— | H | (CH₂)₂—O—C(O)— | 2) | (CH₂)₂—O—C(O)— |
| | CH₂—O— | | CH₂—C(O)—CH₃ | | CH₂—C(O)—CH₃ |
| | C(O)—CH₂— | | | | |
| | CH₂—C(O)— | | | | |
| (7″) | *—(CH₂)ₚ— | *(CH₂)₍ₚ₋₂₎— | *—(CH₂)₍ₚ₋₂₎—S— | *-n-(alkyl- | *—(CH₂)₍ₚ₋₄₎—S— |
| | CH(OH)— | H | (CH₂)₂—O—C(O)— | 2) | (CH₂)₂—[O—C(O)— |
| | CH[C(O)- | | CH₂—C(O)—CH₃ | | CH₂—C(O)—CH₃ |
| | R | | | | |
| (8″) | *—(CH₂)ₚ—S— | *(CH₂)₍ₚ₋₂₎—H | *—(CH₂)₍ₚ₋₂₎—S— | *(CH₂)₍ₚ₋₄₎—H | *—(CH₂)₍ₚ₋₄₎—S— |
| | ALK₃-[O— | | ALK₃—[O—C(O)— | | ALK₃—[O—C(O)— |
| | C(O)—CH₂— | | CH₂—C(O)—CH₃]₂ | | CH₂—C(O)—CH₃]₂ |
| | C(O)—CH₃]₂ | | | | |

More particularly, the stereochemistry of the carbon atoms bearing the radicals R¹, R², R³, R⁴ and R⁵ is of the same (R) or (S) configuration, preferably of (R) configuration.

The PHA copolymer(s) of the invention preferably have a number-average molecular weight ranging from 50 000 to 500 000, more preferentially from 50 000 to 150 000.

The molecular weight may notably be measured by size exclusion chromatography. A method is described below in the examples.

The PHA copolymer(s) of the invention are particularly present in the composition according to the invention in a content ranging from 0.1% to 30% by weight and preferably ranging from 0.1% to 25% by weight relative to the total weight of the composition; particularly 1% to 50% by weight and more particularly ranging from 3% to 40% by weight and preferably ranging from 5% to 35% by weight, more preferably ranging from 10% to 30%, and better ranging from 15% to 20% by weight relative to the total weight of the composition by weight relative to the total weight of the composition.

### Method for Preparing the PHA Copolymer(s):

The methods for preparing the PHA copolymer(s) of the invention are known to those skilled in the art. Mention may notably be made of the use of “functionalizable” PHA-producing microbial strains.

The term “functionalizable” means that the PHA copolymer(s) comprise a hydrocarbon-based chain comprising one or more atoms or groups that are capable of reacting chemically with another reagent—also referred to as “reactive atoms or reactive groups”—to give a Σ covalent bond with said reagent. The reagent is, for example, a compound comprising at least one nucleophilic group and said functionalized hydrocarbon-based chain comprises at least one electrophilic or nucleofugal atom or group, the nucleophilic group(s) reacting with the electrophilic group(s) to covalently 7 graft the reagent. The nucleophilic reagent may also react with one or more unsaturations of the alkenyl group(s) to also lead to grafting by covalent bonding of the functionalized hydrocarbon-based chain with said reagent. The addition reaction may also be radical-based, an addition of Markovnikov or anti-Markovnikov type, or nucleophilic or electrophilic substitution. The addition or condensation reactions may or may not take place via a radical route, with or without the use of catalysts or of enzymes, with heating preferably to a temperature less than or equal to 100° C. or without supplying heat, under a pressure of greater than 1 atm or otherwise, under an inert atmosphere or otherwise, or under oxygen or otherwise.

The term “nucleophilic” refers to any atom or group which is electron-donating by an inductive effect +I and/or a mesomeric effect +M. Electron-donating groups that may be mentioned include hydroxyl, thiol and amino groups.

The term “electrophilic” refers to any atom or group which is electron-withdrawing by an inductive effect −I and/or a mesomeric effect −M. Electron-withdrawing species that may be mentioned include.

The microorganisms producing PHAs of the invention notably bearing a hydrocarbon-based chain may be naturally produced by the bacterial kingdom, such as Cyanobacteria of the order of Nostocales (e.g.: Nostoc muscorum, Synechocystis and Synechococcus) but mainly by the Proteobacteria, for example in the class of:
beta-Proteobacteria, of the order Burkholderiales (Cupriavidus negator synonym Ralstonia eutropha)
alpha-Proteobacteria, of the order Rhodobacteriales (Rhodobacter capsulatus marine and photosynthetic)
gamma-Proteobacteria, of the order Pseudomonales of the family Moraxellaceae (Acinetobacter junii).

Among the microorganisms of the bacterial kingdom, the genera Azotobacter, Hydrogenomomas or Chromatium are the most representative of the PHA-producing organisms.

The organisms which naturally produce PHAs bearing notably a C₃-C₅ hydrocarbon-based chain are notably Proteobacteria, such as gamma-Proteobacteria, and more particularly of the order Pseudomonales of the family Pseudomonas such as Pseudomonas resinovorans, Pseudomonas putida, Pseudomonas fluorescens, Pseudomonas aeruginosa, Pseudomonas citronellolis, Pseudomonas mendocina, Pseudomonas chlororaphis and preferably Pseudomonas putida GPo1 and Pseudomonas putida KT2440, preferably Pseudomonas putida and Pseudomonas putida and in particular Pseudomonas putida GPo1 and Pseudomonas putida KT2440.

Certain organisms may also naturally produce PHAs without belonging to the order of Pseudomonales, such as Commamonas testosteroni which belongs to the class of beta-Proteobacteria of the order Burkholderiales of the family of Comamonadaceae.

The microorganism producing PHAs according to the invention may also be a recombinant strain if a 3-oxidation PHA synthase metabolic pathway is present. The 3-oxidation PHA synthase metabolic pathway is mainly represented by four classes of enzymes, EC: 2.3.1 B2, EC: 2.3.1 B3, EC: 2.3.1 B4 and EC: 2.3.1 B5.

The recombinant strain may be from the Bacteria kingdom, for instance Escherichia coli, or from the Plantae kingdom, for instance Chlorella pyrenoidosa (International Journal of Biological Macromolecules, 116, 552-562 “Influence of nitrogen on growth, biomass composition, production, and properties of polyhydroxyalkanoates (PHAs) by microalgae”) or from the Fungi kingdom, for instance Saccharomyces cerevisiae or Yarrowia lipolytica: Applied Microbiology and Biotechnology 91, 1327-1340 (2011) “Engineering polyhydroxyalkanoate content and monomer composition in the oleaginous yeast Yarrowia lipolytica by modifying the β-oxidation multifunctional protein”).

Use may also be made of genetically modified microorganisms, which may make it possible, for example, to increase the production of PHA, and/or to increase the oxygen consumption capacity, and/or to reduce the autolysis and/orto modify the monomer ratio.

It is known that, for PHAs, a large portion of the total production cost is devoted to the culture medium and mainly to the substrate/carbon source. Use may thus be made of genetically modified microorganisms using a smaller amount of nutrient (carbon source) for their growth, for example microorganisms that are photo-autotrophic by nature, i.e. using light and CO₂ as main energy source.

The copolymer may be obtained in a known manner by biosynthesis, for example with the microorganisms belonging to the genus Pseudomonas, such as Pseudomonas resinovorans, Pseudomomonas putida, Pseudomonas fluorescens, Pseudomonas aeruginosa, Pseudomonas citronellolis, Pseudomonas mendocina, Pseudomonas chlororaphis and preferably Pseudomonas putida; and with a carbon source which may be a C₂-C₂₀, preferably C₆-C₁₈, carboxylic acid, such as acetic acid, propionic acid, butyric acid, hexanoic acid, heptanoic acid, octanoic acid, nonanoic acid, dodecanoic acid; a saccharide, such as fructose, maltose, lactose, xylose, arabinose, etc.); an n-alkane, such as hexane, octane or dodecane; an n-alcohol, such as methanol, ethanol, octanol or glycerol; methane or carbon dioxide.

The biosynthesis may optionally be performed in the presence of an inhibitor of the β-oxidation pathway, such as acrylic acid, methacrylic acid, propionic acid, cinnamic acid, salicylic acid, pentenoic acid, 2-butynoic acid, 2-octynoic acid or phenylpropionic acid, and preferably acrylic acid.

According to one embodiment, the process for preparing the PHAs of the invention uses microbial cells which produce PHAs via genetically modified microorganisms (GMOs). The genetic modification may increase the production of PHA, increase the oxygen absorption capacity, increase the resistance to the toxicity of solvents, reduce the autolysis, modify the ratio of the PHA comonomers, and/or any combination thereof. In some of these embodiments, the modification of the comonomer ratio of the unit (A) increases the amount of predominant monomer versus (B) of the PHA of the invention which is obtained. In another embodiment, the PHA-producing microbial cells reproduce naturally.

By way of example, a genetically modified microbial strain producing PHA that is functionalizable or comprising a reactive group that may be mentioned is Pseudomonas entomophila LAC23 (Biomacromolecules. 2014 Jun. 9; 15(6):2310-9. doi: 10.1021/bm500669s).

It is also possible to use genetically modified microorganisms which produce phenylvaleric-co-3-hydroxydodecanoic copolymers (Sci. China Life Sci., Shen R., et al., 57 No. 1, (2014) with a strain such as Pseudomonas entomophila LAC23.

Nutrients, such as water-soluble salts based on nitrogen, phosphorus, sulfur, magnesium, sodium, potassium and iron, may also be used for the biosynthesis.

The appropriate known conditions of temperature, pH and dissolved oxygen (DO) can be used for the culturing of the microorganisms.

The microorganisms may be cultured according to any known method of culturing, such as in a bioreactor in continuous or batch mode, in fed or unfed mode.

The biosynthesis of the polymers used according to the invention is notably described in the article “Biosynthesis and Properties of Medium-Chain-Length Polyhydroxyalkanoates with Enriched Content of the Dominant Monomer”, Xun Juan et al., Biomacromolecules 2012, 13, 2926-2932, and in patent application WO 2011/069244.

The microbial strains producing PHA which is functionalizable or comprising a reactive group, as defined previously, are, for example, of the genus Pseudomonas such as P. cichorii YN2, P. citronellolis, P. jessenii, and more generally with species of Pseudomonas putida such as Pseudomonas putida GPo1 (synonym of Pseudomonas oleovorans), P. putida KT2442, P. putida KCTC 2407, P. putida BM01.

The carbon source(s):

One means for gaining access to the PHAs of the invention is to introduce one or more organic compounds into the culture medium, this or these organic compounds each representing a carbon source preferably chosen from alkanes, alkenes, alcohols, carboxylic acids and a mixture thereof.

In one embodiment, the organic compound(s) will preferably be chosen from alcohols, carboxylic acids and a mixture thereof.

The carbon source(s) may be classified in two categories:

1) Carbon Source Via One or More Organic Compounds Introduced into the Medium:

One means for gaining access to the PHA(s) of the invention is to introduce one or more organic compounds into the culture medium, each organic compound being a carbon source preferably chosen from alkanes, alkenes, alcohols, carboxylic acids and mixtures thereof.

According to a particular embodiment of the invention, the organic compound(s) are chosen from alcohols, in particular (C₅-C₂₀)alkanols, and/or carboxylic acids, in particular (C₅-C₂₀)alkanoic acids.

The carbon source(s) may be classified into three groups according to their intended use:
group A: the organic compound may aid the growth of the productive strain and aid the production of PHA structural linked to the organic compound.
group B: the organic compound may aid the growth of the strain but does not participate in the production of PHA structurally linked to the organic compound.
group C: the organic compound does not participate in the growth of the strain.

Such microbiological processes are known to those skilled in the art, notably in the scientific literature. Mention may be made of: International Journal of Biological Macromolecules 28, 23-29 (2000); The Journal of Microbiology, 45, No. 2, 87-97, (2007).

According to one variant, the integration of the substrate that is structurally linked to the reactive atom(s) or to the reactive group(s) of the PHA of the invention is introduced directly into the medium as sole carbon source in a medium suitable for microbial growth. (Example: group A for P. putida GPo1: alkenoic acid, notably terminal).

According to another variant, the integration of the substrate that is structurally linked to the reactive atom(s), notably halogen, or to the reactive group(s) of the PHA of the invention is introduced into the medium as carbon source with a second carbon source as co-substrate which is also structurally linked to the PHA, in a medium suitable for microbial growth. (Example: group B for P. putida GPo1: haloalkanoic acids which are preferably terminal, such as terminal bromoalkanoic acids).

According to yet another variant, the integration of the substrate that is structurally linked to the reactive atom(s), notably halogen, or to the reactive group(s) of the PHA of the invention may be introduced directly into the medium as carbon source with a second carbon source as co-substrate which is also structurally linked to the PHA and a third carbon source as co-substrate which is not structurally linked to the PHA, in a medium suitable for microbial growth. (Example: group C glucose or sucrose).

In one embodiment, the β-oxidation pathway inhibitor is acrylic acid, 2-butynoic acid, 2-octynoic acid, phenylpropionic acid, propionic acid, trans-cinnamic acid, salicylic acid, methacrylic acid, 4-pentenoic acid or 3-mercaptopropionic acid, preferably acrylic acid.

In one embodiment of the first aspect, the functionalized fatty acid is a functionalized hexanoic acid, functionalized heptanoic acid, functionalized octanoic acid, functionalized nonanoic acid, functionalized decanoic acid, functionalized undecanoic acid, functionalized dodecanoic acid or functionalized tetradecanoic acid.

The functionalization may be introduced by means of an organic compound chosen from precursors of the alcohol and/or carboxylic acid category, notably:
for functionalization of the PHA(s) with a branched alkyl group: see, for example Applied and Environmental Microbiology,. 60, No. 9, 3245-325 (1994);
for functionalization of the PHA(s) with a linear alkyl group comprising a terminal cyclohexyl unit: see, for example doi.org/10.1016/S0141-8130(01)00144-1;
for functionalization of the PHA(s) with an unsaturated alkyl group which is preferably terminal: see, for example doi.org/10.1021/bm8005616);
for functionalization of the PHA(s) with a linear alkyl group comprising a halogen preferably at the end of the hydrocarbon-based chain (doi.org/10.1021/ma00033a002);
for functionalization of the PHA(s) with a (hetero)aromatic alkyl group, for example phenyl, benzoyl, phenoxy, see, for example J. Microbiol. Biotechnol., 11, 3, 435-442 (2001);
for functionalization of the PHA(s) with a linear alkyl group comprising a heteroatom notably at the end of the hydrocarbon-based chain, see, for example DOI 10.1007/s00253-011-3099-4;
for functionalization of the PHA(s) with a linear alkyl group comprising a cyano function notably at the end of the hydrocarbon-based chain, see, for example doi.org/10.1111/j.1574-6968.1992.tb05839.x;
for functionalization of the PHA(s) with a linear alkyl group comprising an epoxy function notably at the end of the hydrocarbon-based chain, see, for example doi.org/10.1016/Si381-5148(97)00024-2;

The review International Microbiology 16:1-15 (2013) doi:10.2436/20.1501.01.175) also mentions the majority of the functionalized native PHAs.

In a particular embodiment of the invention, the fatty acid from group A is chosen from 11-undecenoic acid, 10-epoxyundecanoic acid, 5-phenylvaleric acid, citronellol and 5-cyanopentanoic acid, preferably 11-undecenoic acid.

In a particular embodiment of the invention, the fatty acid from group B is chosen from halooctanoic acids such as 8-bromooctanoic acid.

In a particular embodiment of the invention, the carbon source from group C is a monosaccharide, preferably glucose.

2) Carbon Source in the Presence of Oxidation Inhibitor Introduced into the Medium:

Another aspect of the invention is the use of the PHA-producing microbial strains in a medium that is suitable for microbial growth, said medium comprising: a substrate which is structurally linked to the PHA(s); at least one carbon source which is not structurally linked to the PHA(s); and at least one oxidation and notably β-oxidation pathway inhibitor. This allows the growth of the microbial cells to take place in said medium, the microbial cells synthesizing the PHA polymer(s) of the invention; preferably copolymer particularly containing more than 95% of identical units, which has a comonomer ratio of unit (A) and of unit (B) which differs from that obtained in the absence of the μ-oxidation pathway inhibitor.

The schemes below illustrate, by way of example, functionalization with groups A) or B) on R¹ of PHA copolymers according to the invention:
* either from PHA with an unsaturated hydrocarbon-based chain (a) which reacts with a nucleophilic reagent YH according to the following scheme 1 to lead to compound (b):

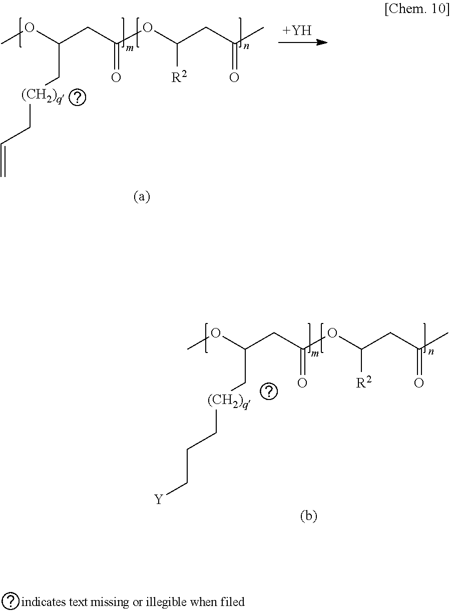

in which Scheme 1:
   R², m and n are as defined previously;
   Y represents a group chosen from R³—C(X)—C(R⁴)(R⁵)—C(X′)—[Y]ₙ-L-X″—* and R³—C(X)—C(—[Y]ₙ-L-X″—*)(R⁴)—C(X)—R⁶ with R³, R⁴, R⁵, R⁶, n, Y, X, X′ and * as defined previously, L representing a divalent linear or branched hydrocarbon-based chain comprising from 1 to 20 carbon atoms optionally substituted notably with one or more hydroxyl groups and/or optionally interrupted with one or more a′) heteroatoms such as O, S, N(Rₐ) and Si(R_{b})(R_{c}), b′) S(O)ᵣ, (thio)carbonyl, c′) or combinations of a′) with b′) such as (thio)ester, (thio)amide, (thio)urea, sulfonamide with r being equal to 1 or 2, Rₐ being as defined previously; preferably, Rₐ represents a hydrogen atom, R_{b} and R_{c} being as defined previously for Rₐ; in particular, L is chosen from linear or branched (C₁-C₁₀)alkylene or linear or branched (C₂-C₁₀)alkenylene, preferably linear (C₁-C₆)alkylene optionally interrupted with O, S or ester, and X″ being as defined for X, and preferably represents S;
   q′ represents an integer inclusively between 2 and 20, preferably between 3 and 10, more preferentially between 4 and 8 such as 6, better still between 3 and 8, preferably between 4 and 6, such as 5.

Other reactions may be performed using double or triple unsaturations such as Michael or Diels-Alder additions, radical reactions, catalytic (notably with Pd or Ni) or non-catalytic hydrogenation reactions, halogenation reactions, notably with bromine, hydration reactions or oxidation reactions, which may or may not be controlled, and reactions on electrophiles.

According to a particular embodiment of the invention, the PHA copolymers comprise
a linear or branched, saturated hydrocarbon-based chain R¹, substituted and optionally interrupted with atoms or groups as defined previously for R¹, comprising in total between 5 and 30 carbon atoms, preferably between 6 and 20 carbon atoms and more particularly between 7 and 11 carbon atoms, said chain being substituted with at least one group A) and/or B) as defined previously; and
a hydrocarbon-based chain R² representing a linear or branched (C₃-C₂₀)alkenyl, particularly (C₅-C₁₄)alkenyl and more particularly (C₇-C₁₀)alkenyl radical, which is preferably linear and comprising only one unsaturation at the chain end, in particular —[CR⁴(R⁵)]_{q}—C(R⁶)═C(R⁷)—R⁸ with R⁴, R⁵, R⁶, R⁷ and R⁸, which may be identical or different, representing a hydrogen atom or a (C₁-C₄)alkyl group such as methyl, preferably a hydrogen atom, and q represents an integer inclusively between 2 and 20, preferably between 3 and 10, more preferentially between 4 and 8 such as 6, such as —[CH₂]_{q}—CH═CH₂ and q represents an integer inclusively between 3 and 8, preferably between 4 and 6, such as 5,
   said chain R² comprising between 1% and 99%, preferentially between 2% and 50% and even more preferentially between 3% and 40% of unsaturations, and even more particularly between 3% and 30% of unsaturations, better still between 5% and 20% of unsaturations. According to this particular embodiment of the invention in which the PHA copolymers comprise unsaturations, these unsaturations may be chemically modified:
A) via addition reactions, such as radical additions, Michael additions, electrophilic additions, Diels-Alder, halogenation, hydration or hydrogenation reaction, and preferably hydrothiolation reaction with particles, chemical compounds or polymers.

In particular, the hydrothiolation reactions may be performed in the presence of a thermal initiator, a redox initiator or a photochemical initiator and of an organic compound bearing a sulfhydryl group, notably chosen from:
linear, branched, cyclic or aromatic alkanethiols including 1 to 14 carbon atoms, such as methane-, ethane-, propane-, pentane-, cyclopentane-, hexane-, cyclohexane-, heptane-, octane-, phenylethane-, 4-tert-butylphenylmethane- or 2-furanmethane-thiol, preferably hexane-, cyclohexane-, heptane-, octane-, phenylethane-, 4-tert-butylphenylmethane- or 2-furanmethane-thiol;
organosiloxanes bearing a thiol function, such as (3-mercaptopropyl)trimethoxysilane, (3-mercaptopropyl)methyldimethoxysilane, 2-(triethoxysilyl)ethanethiol or mercaptopropyl-isobutyl-POSS;
thiolated silicone oils, notably those described in the document DOI: 10.1016/j.actbio.2015.01.020);
thiolated oligomers or polymers bearing a reactive function, such as an amine, an alcohol, an acid, a halogen, a thiol, an epoxide, a nitrile, an isocyanate, a heteroatom, preferably cysteine, cysteamine, N-acetylcysteamine, 2-mercaptoethanol, 1-mercapto-2-propanol, 8-mercapto-1-octanol, thiolactic acid, thioglycolic acid, 3-mercaptopropionic acid, 11-mercaptoundecanoic acid, polyethylene glycol dithiol, 3-mercaptopropionitrile, 1,3-propanedithiol, 4-cyano-1-butanethiol, 3-chloro-1-propanethiol, 1-thio-β-D-glucose tetraacetate; and
thiols which may be obtained from disulfide reduction, such as phenyl disulfide or furfuryl disulfide.

Examples of initiators that may be mentioned include: tert-butyl peroxy-2-ethylhexanoate, cumene perpivalate, tert-butyl peroxylaurate, benzoyl peroxide, lauroyl peroxide, octanoyl peroxide, di-tert-butyl peroxide, tert-butylcumyl peroxide, dicumyl peroxide, 2,2′-azobisisobutyronitrile, 2,2′-azobis(2-methylbutyronitrile), 2,2′-azobis(2,4-dimethylvaleronitrile), 2,2′-azobis(4-methoxy-2,4-dimethylvaleronitrile), 1,1-bis(tert-butylperoxy)-3,3,5-trimethylcyclohexane, 1,1-bis(tert-butylperoxy)cyclohexane, 1,4-bis(tert-butylperoxycarbonyl)cyclohexane, 2,2-bis(tert-butylperoxy)octane, n-butyl 4,4-bis(tert-butylperoxy)valerate, 2,2-bis(tert-butylperoxy)butane, 1,3-bis(tert-butylperoxyisopropyl)benzene, 2,5-dimethyl-2,5-bis(tert-butylperoxy)hexane, 2,5-dimethyl-2,5-bis(benzoylperoxy)hexane, di-tert-butyl diperoxyisophthalate, 2,2-bis(4,4-di-tert-butylperoxycyclohexyl)propane, di-tert-butyl peroxy-α-methylsuccinate, di-tert-butyl peroxydimethylglutarate, di-tert-butyl peroxyhexahydroterephthalate, di-tert-butyl peroxyazelate, 2,5-dimethyl-2,5-bis(tert-butylperoxy)hexane, diethylene glycol bis(tert-butylperoxycarbonate), di-tert-butyl peroxytrimethyladipate, tris(tert-butylperoxy)triazine, vinyltris(tert-butylperoxy)silane phenothiazine, tetracene, perylene, anthracene, 9,10-diphenylanthracene, thioxanthone, benzophenone, acetophenone, xanthone, fluorenone, anthraquinone, 9,10-dimethylanthracene, 2-ethyl-9,10-dimethyloxyanthracene, 2,6-dimethylnaphthalene, 2,5-diphenyl-1,3,4-oxadiazole, xanthopinacol, 1,2-benzanthracene, 9-nitroanthracene. Each of these initiators may be used alone or in combination with others.

The chemical reactions mentioned previously are known to those skilled in the art. Mention may notably be made of the following documents: Synthesis and preparation of PHAs modified with polyethylene glycol dithiol: 10.1021/acs.biomac.9b00479; Biomacromolecules, 19, 3536-3548 (2018); Synthesis and preparation of PHAs modified with mercaptohexanol: 10.1021/acs.biomac.8b01257; Biomacromolecules, 20, 2, 645-652 (2019); Synthesis and preparation of PHAs modified with hydroxycinnamic acid sulfate, and zosteric acid: 10.1021/bm049962e; Biomacromolecules, 5, 4, 1452-1456 (2004); Radical addition of methyl methacrylate to a PHOUn: 10.1002/1521-3935(20010701)202:11<2281::AID-MACP2281>3.0.CO; 2-9; Macromolecular Chemistry and Physics, vol. 202, 11, 2281-2286 (2001); Synthesis and preparation of PHAs modified with a polysilsesquioxane (POSS): 10.1016/j.polymer.2005.04.020; Polymer Vol. 46, 14, 5025-5031 (2005); Grafting of thio-beta-glucose onto unsaturated side chains: 1022-1336/99/0202-0091$17.50+0.50/0; Macromol. Rapid Commun., 20, 91-94 (1999);

and/or
B) via oxidation reactions, which may or may not be controlled, for example with permanganates of a concentrated or dilute alkaline agent, or ozonolysis, oxidation in the presence of a reducing agent, making it possible to obtain novel materials bearing hydroxyl, epoxide or carboxyl groups in the terminal position of the side chains.

The chemical reactions mentioned previously are known to those skilled in the art. Mention may notably be made of the following documents: 10.1021/bm049337; Biomacromolecules, vol. 6, 2, 891-896 (2005); 10.1016/S0032-3861(99)00347-X; Polymer, vol. 41, 5, 1703-1709 (2000); 10.1021/ma9714528 and 10.1016/S1381-5148(97)00024-2; Macromolecules, 23, 15, 3705-3707 (1990); 10.1016/S0032-3861(01)00692-9; Polymer, vol. 43, 4, 1095-1101 (2002); 10.1016/S0032-3861(99)00347-X; Polymer, vol. 41, 5, 1703-1709 (2000); and 10.1021/bm025728h; Biomacromolecules, vol. 4, 2, 193-195 (2003).
or from PHA with a hydrocarbon-based chain bearing an epoxide group (c) which reacts with a nucleophilic reagent YH according to the following scheme 2 to lead to compound (d):

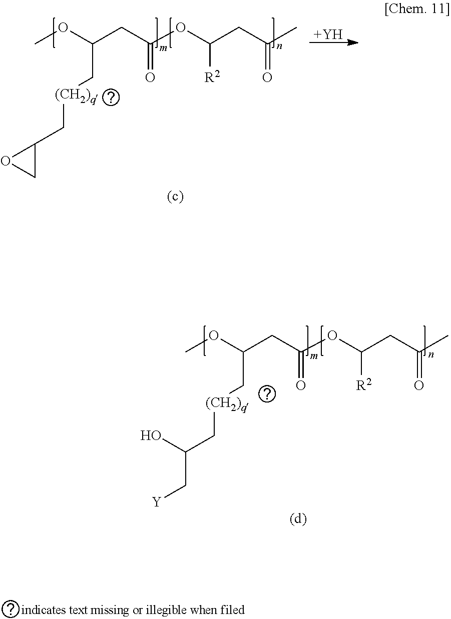

in which Scheme 2 Y, m, n, q′ and R² are as defined in Scheme 1.

The epoxide structure may be obtained via a conventional method known to those skilled in the art, whether via biotechnological processes or via chemical processes such as oxidation of unsaturation as mentioned previously.

Mention may notably be made of the following documents:
Preparation of PHA bearing charges starting with diethanolamine: 10.1021/bm8005616, Biomacromolecules, vol. 9, 8 2091-2096 (2008);
Preparation of PHA bearing charges starting with sodium 3-mercapto-1-propanesulfonate: 10.1021/acs.biomac.9b00870 Biomacromolecules, vol. 20, 9, 3324-3332 (2019);
Preparation of PHA including a native epoxide unit: 10.1016/S1381-5148(97)00024-2); Reactive and Functional Polymers, vol. 34, 1, 65-77 (1997).
or from PHA with a hydrocarbon-based chain bearing a nucleofugal group (e) which reacts with a nucleophilic reagent YH according to the following scheme 3 to lead to compound (f):

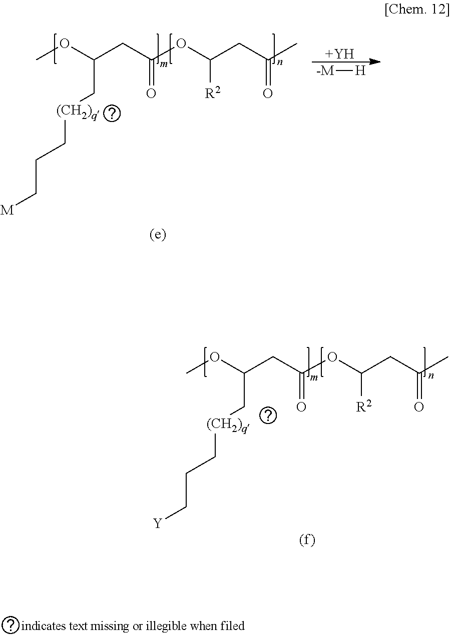

in which Scheme 3 Y, m, n, q′ and R² are as defined in Scheme 1. M corresponds to an organic or inorganic nucleofugal group, which may be substituted with a nucleophilic group; preferably, said nucleophile is a heteroatom which is electron-donating via the +1 and/or +M effect such as 0, S or N. Preferably, the nucleofugal group M is chosen from halogen atoms such as Br, and mesylate, tosylate or triflate groups. This is a reaction known to those skilled in the art. Mention may be made, for example, of the following document: 10.1016/j.ijbiomac.2016.11.118, International Journal of Biological Macromolecules, vol. 95, 796-808 (2017).
or from PHA with a hydrocarbon-based chain bearing a cyano group which reacts with a nucleophilic reagent YH according to the following scheme 4:

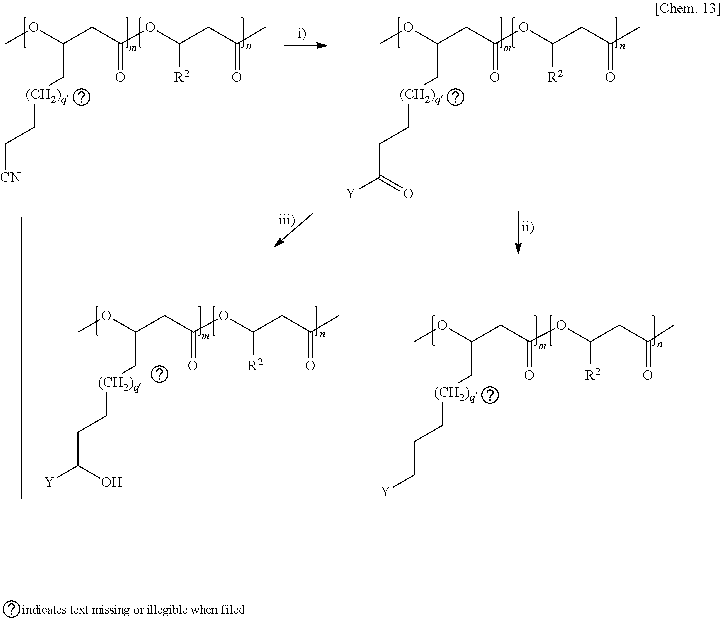

in which Scheme 4 Y, m, n, q′ and R² are as defined in Scheme 1.

In a first step i), the PHA copolymer bearing a side chain containing a cyano or nitrile group reacts with an organo-alkali metal or organomagnesium compound Y-MgHal, Y—Li or Y—Na, followed by hydrolysis to give the PHA copolymer bearing a side chain containing a group Y grafted with a ketone function. The ketone function may be converted into a thio ketone by thionation, for example with S8 in the presence of amine, or with Lawesson's reagent. Said thio ketone, after total reduction ii) (for example by Clemmensen reduction) leads to the PHA copolymer bearing a side chain containing a group Y grafted with an alkylene group. Alternatively, said thio ketone may undergo a controlled reduction iii) with a conventional reducing agent to give the PHA copolymer bearing a side chain containing a group Y grafted with a hydroxyalkylene group. PHA copolymers with a hydrocarbon-based chain bearing a nitrile function are prepared via conventional methods known to those skilled in the art. Mention may be made, for example, of the document: 10.1016/0378-1097(92)90311-B, FEMS Microbiology Letters, vol. 103, 2-4, 207-214 (1992).
or from PHA with a hydrocarbon-based chain at the end of the chain (g) which reacts with a reagent HR′¹ according to the following scheme 5 to lead to compound (h):

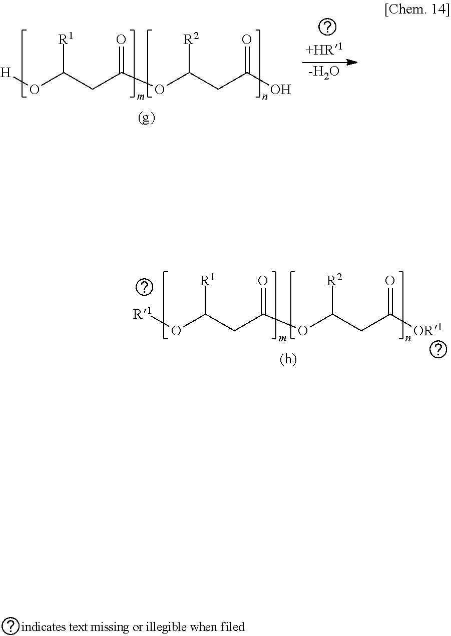

in which Scheme 5 R¹, R², m, n and Y are as defined previously, and R′¹ represents a hydrocarbon-based chain chosen from i) linear or branched (C₁-C₂₀)alkyl, ii) linear or branched (C₂-C₂₀)alkenyl, iii) linear or branched (C₂-C₂₀)alkynyl; iii) preferably, the hydrocarbon-based group is linear; said hydrocarbon-based chain being substituted with one or more atoms or groups chosen from: a) halogens such as chlorine or bromine, b) hydroxyl, c) thiol, d) (di)(C₁-C₄)(alkyl)amino, e) (thio)carboxyl, f) (thio)carboxamide —C(O)—N(Rₐ)₂ or —C(S)—N(Rₐ)₂, f) cyano, g) iso(thio)cyanate, h) (hetero)aryl such as phenyl or furyl, and i) (hetero)cycloalkyl such as anhydride, or epoxide, j) a cosmetic active agent chosen from coloured or uncoloured, fluorescent or non-fluorescent chromophores such as those derived from optical brighteners, or chromophores derived from UVA and/or UVB screening agents, and anti-ageing active agents, k) A) as defined previously, I) B) as defined previously or m) Y as defined previously.

These chain-end grafts onto PHA polymers are known to those skilled in the art. Mention may be made, for example, of the following documents:
Preparation of PHA oligomers by thermal degradation: 10.1021/bm0156274; Biomacromolecules, vol. 3, 1, 219-224 (2002);
Preparation of PHA oligomers by transesterification: 10.1021/ma011420r, Macromolecules, vol. 35, 3, 684-689 (2002);
Preparation of PHA oligomers by hydrolysis: 10.1016/0032-3861(94)90590-8 Polymer_vol. 35, 19, 4156-4162 (1994);
Preparation of PHA oligomers by methanolysis: 10.1021/bm060981t, Biomacromolecules, vol. 8, 4, 1255-1265 (2007).

Mention may also be made of other methods known to those skilled in the art:
Synthesis and characterization of PHA grafted with ascorbic acid: 10.1016/j.ijbiomac.2018.11.052; International Journal of Biological Macromolecules, vol. 123: 7 (2019);
Preparation of PHB-b-PHO copolymers by polycondensation with divinyl adipate catalysed with a lipase: 10.1021/bm9011634, Biomacromolecules, vol. 10, 12, 3176-3181 (2009);
Synthesis of PHB-b-PHO copolymers coupled via a diisocyanate junction: 10.1021/ma012223v; Macromolecules, vol. 35, 13, 4946-4950 (2002);
Preparation of PHO oligomers on chitosan by condensation between the carboxylic acid end of the PHO and the amine functions of the chitosan: 10.1002/app.24276; Journal of Applied Polymer Science, vol. 103, 1, (2006);
Transesterification of PHAs with propargyl alcohol in order to produce PHA oligomers that are modifiable by “click” chemistry: 10.1016/j.reactfunctpolym.2011.12.005; Reactive and Functional Polymers, vol. 72, 2, 160-167 (2012);
Preparation of PHO-b-PCL copolymer: 10.1002/mabi.200400104; Macromolecular Bioscience, vol. 4, 11 (2004);
Preparation of PHO-b-PEG copolymer: 10.1002/macp.201000562; Macromolecular Chemistry and Physics; vol. 212, 3, (2010);
Epoxidation of chain-end unsaturation and chain-end grafting of acid: 10.14314/polimery.2017.317; Polimery, vol. 62, 4, 317-322 (2017);
Grafting of organosiloxane unit at chain end onto PHA: 10.1016/j.reactfunctpolym.2014.09.008; Reactive and Functional Polymers, vol. 84, 53-59 (2014).
   or from PHA with a hydrocarbon-based chain bearing a reactive group (i) which reacts with a nucleophile or an electrophile according to the following scheme 6 to lead to compound (j):

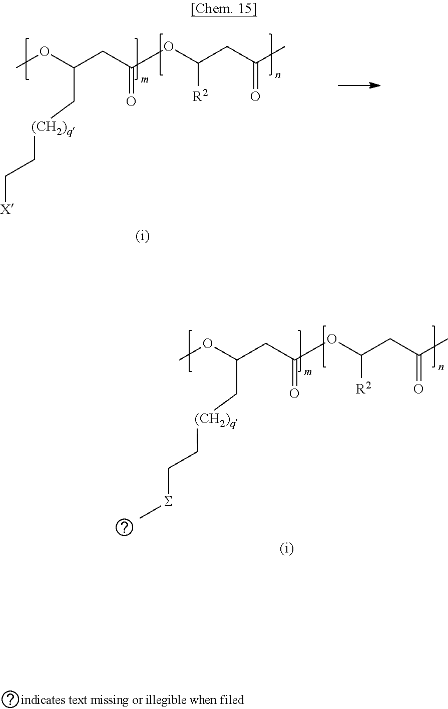

in which Scheme 6 R², m, n and Y are as defined previously, and
R′¹ represents a hydrocarbon-based chain chosen from i) linear or branched (C₁-C₂₀)alkyl, ii) linear or branched (C₂-C₂₀)alkenyl, iii) linear or branched (C₂-C₂₀)alkynyl, iii) preferably the hydrocarbon-based group is linear; said hydrocarbon-based chain being substituted with one or more groups chosen from A) as defined previously, 1) as defined previously and Y as defined previously;
X′ represents a reactive atom or group that is capable of reacting with an electrophilic [symbol] or nucleophilic [symbol] atom or group to create a Σ covalent bond; if X′ is an electrophilic or nucleofugal group, then it can react with a reagent R′¹- [symbol] ; if X′ is a nucleophilic group [symbol] , then it can react with R′¹- [symbol] to create a Σ covalent bond;

By way of example, the Σ covalent bonds or bonding group that may be generated are listed in the table below, from condensation of electrophiles with nucleophiles:

TABLE 1

| Electropliles [symbol] | Nucleophiles [symbol] | Covalent bonds Σ |
|---|---|---|
| Activated esters* | Amines | Carboxamides |
| Acyl azides ** | Amines | Carboxamides |
| Acyl halides | Amines | Carboxamides |
| Acyl halides | Alcohols | Esters |
| Acyl cyanides | Alcohols | Esters |
| Acyl cyanides | Amines | Carboxamides |
| Alkyl halides | Amines | Alkylamines |
| Alkyl halides | Carboxylic acids | Esters |
| Alkyl halides | Thiols | Thioesters |
| Alkyl halides | Alcohols | Ethers |
| Sulfonic acids and salts thereof | Thiols | Thioethers |
| Sulfonic acids and salts thereof | Carboxylic acids | Esters |
| Sulfonic acids and salts thereof | Alcohols | Ethers |
| Anhydrides | Alcohols | Esters |
| Anhydrides | Amines | Carboxamides |
| Aryl halides | Thiols | Thioethers |
| Aryl halides | Amines | Arylamines |
| Aziridines | Thiols | Thioethers |
| Carboxylic acids | Amines | Carboxamides |
| Carboxylic acids | Alcohols | Esters |
| Carbodiimides | Carboxylic acids | N-acylureas |
| Diazoalkanes | Carboxylic acids | Esters |
| Epoxides | Thiols | Thioethers |
| Haloacetamides | Thiols | Thioethers |
| Imide esters | Amines | Amidines |
| Isocyanates | Amines | Ureas |
| Isocyanates | Alcohols | Urethanes |
| Isothiocyanates | Amines | Thioureas |
| Maleimides | Thiols | Thioethers |
| Sulfonic esters | Amines | Alkylamines |
| Sulfonic esters | Thiols | Thioethers |
| Sulfonic esters | Carboxylic acids | Esters |
| Sulfonic esters | Alcohols | Ethers |
| Sulfonyl halides | Amines | Sulfonamides |

*activated esters of general formula —CO—LG with LG representing a leaving group such as oxysuccinimidyl, oxybenzotriazolyl, optionally substituted aryloxy:
**acyl azides can rearrange to give isocyanates

It is also possible, starting with a PHA functionalized on a side chain, to perform chain-end grafting in a second stage as described in Scheme 7. The reverse is also true, in which the chain-end grafting may be performed in a first stage, followed by performing functionalization of a functionalizable side chain in a second stage.
or from PHA (j) which reacts at the end of the chain with a reagent HR′¹ according to the following scheme 7 to lead to compound (k):

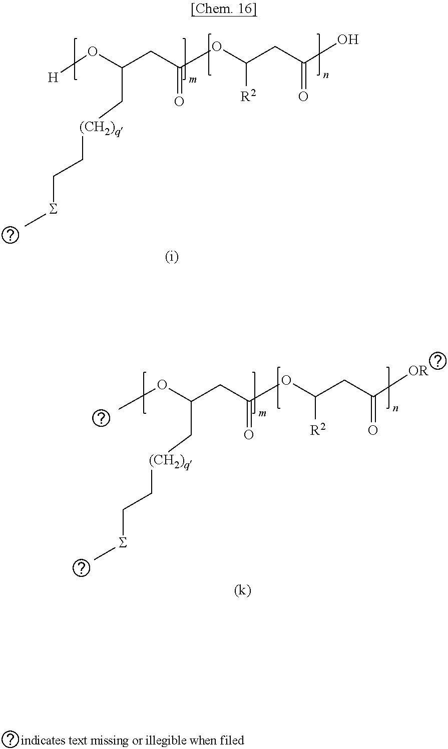

in which Scheme 7 R², m, n and Σ are as previously defined, and R′¹ represents a hydrocarbon-based chain chosen from i) linear or branched (C₁-C₂₀)alkyl, ii) linear or branched (C₂-C₂₀)alkenyl, iii) linear or branched (C₂-C₂₀)alkynyl, iii) preferably, the hydrocarbon-based group is linear; said hydrocarbon-based chain being substituted with one or more groups chosen from A) as defined previously, B) as defined previously and Y as defined previously.

All these chemical reactions are known to those skilled in the art. Mention may be made, for example, of the following documents:
Synthesis and preparation of PHAs modified with thiol-ene followed by reaction on the new grafted function: 10.1021/ma0304426; Macromolecules, vol. 37, 2, 385-389 (2004);
Grafting of PEG and of PLA onto PHAs functionalized with acids: 10.1002/marc.200900803 and 10.1002/mabi.200390033;
Synthesis and preparation of PHAs modified with polyethylene glycol dithiol: 10.1021/acs.biomac.9b00479.

According to a particular embodiment, the PHAs according to the invention are obtained by chemical modification in one or more steps of PHAs (I) which contain at least one unit A′ and optionally at least one unit B′ as defined below:

—[—O—CH(R¹)—CH₂—C(O)—]— unit (A′)

—[—O—CH(R²)—CH₂—C(O)—]— unit (B′)

in which:
R′¹ represents a) a linear or branched, preferably linear, saturated hydrocarbon-based chain terminated with an OH radical, or with an epoxy radical or with a halogen atom or b) an unsaturated linear or branched, preferably saturated, more preferentially ethylenic hydrocarbon-based chain whose double bond is terminal;
R′² preferably represents a linear or branched alkyl radical comprising from 3 to 30 carbon atoms, preferably from 3 to 20 carbon atoms, and more preferentially from 3 to 10 carbon atoms;

According to one embodiment, the radicals R¹ of the PHAs of the invention are obtained by chemical modification of radicals R′¹ of PHAs of formula (I).

According to one embodiment, the reactions performed to give the PHAs of the invention from the PHAs (I) described above are preferably chosen from metathesis, O-alkylation, transesterification, nucleophilic addition, nucleophilic substitution and thiolene reactions.

According to one embodiment, the reagents used to transform the radicals R′¹ into radicals R¹ are chosen from esters R—O—C(O)—O—C(O)—CH₃ with R representing a C₁-C₆ alkyl such as t-butyl, halogenated compounds such as Hal-CH₂—C(O)—CH₂—C(O)—OR′ with Hal representing a halogen such as Cl, and R′ representing a C₁-C₄ alkyl such as ethyl, succinyl acetone CH₃—C(O)—CH₂—C(O)—CH₂—CH₂—C(O)—OH, diketones CH₃—C(O)—CH₂—C(O)—R″ with R″ representing a C₁-C₆ alkyl radical.

According to a preferred embodiment of the invention, the PHAs are obtained by chemical reaction of PHAs of formula (I′) such that the radicals R′² denote C₃-C₃₀ alkyl radicals, preferably C₃-C₂₀, more preferentially C₃-C₁₀.

According to one embodiment, the precursor PHAs of formula (I) are obtained from several carbon sources, preferably from two carbon sources, said several carbon sources being preferably chosen from saturated C₃-C₁₆ carboxylic acids such as nonanoic acid and unsaturated C₃-C₁₆ carboxylic acids such as undecylenic acid.

### The Composition:

Another subject of the invention is a composition, preferably a cosmetic composition, comprising a) one or more PHA bearing acetoacetate group(s) and derivative(s) as defined previously.

According to a particular embodiment of the invention, the composition also comprises b) one or more crosslinking agents.

### b) The Crosslinking Agents

The composition of the invention may comprise one or more crosslinking agents.

For the purposes of the invention, the term “crosslinking agent” means a compound that is capable of establishing with at least one acetoacetate function of the (co)polymer under consideration according to the invention:
at least one covalent bond,
at least one donor-acceptor (dative) bond, and/or
at least one coordination bond and thus of crosslinking this (co)polymer.

Preferably, the term “crosslinking agent” denotes a compound that is capable of establishing at least one covalent bond with a function A or B of the PHA according to the invention and thus of crosslinking said PHA.

For the purposes of the present invention, it is understood that the terms “crosslinking agent” and “crosslinker” are equivalent.

According to a particular embodiment, the crosslinking agent(s) b) are chosen from compounds bearing amine, thiol, alcohol, acrylate, and/or carbonyl functions such as a ketone or aldehyde function.

A crosslinking agent may also denote a metal alkoxide or a metal salt or a rare-earth metal derivative.

Thus, according to a particular embodiment, the crosslinking agent is chosen from (poly)amino, (poly)thiolated and/or (poly)hydroxylated, (poly)carbonylated, (poly)acrylate compounds and mixtures thereof, and preferably chosen from (poly)amino and (poly)thiolated compounds, notably as detailed below. Thus, according to a particular embodiment, the crosslinking agent is chosen from (poly)amino, (poly)thiolated and/or (poly)hydroxylated, (poly)carbonylated, (poly)acrylate, and/or metal alkoxide, metal (poly)(hydroxy)(C₁-C₆)alkylcarboxylate, rare-earth metal derivatives and mixtures thereof, and preferably chosen from (poly)amino and (poly)thiolated compounds, notably as detailed below.

The term “(poly)amine, (poly)thiolated and/or (poly)hydroxylated, (poly)carbonyl and (poly)acrylate compounds” means that the compounds include at least one amine, thiol and/or hydroxyl, carbonyl such as a ketone or aldehyde function, or acrylate function, respectively.

The metal alkoxide compounds, metal (poly)(hydroxy)(C₁-C₆)alkylcarboxylates and rare-earth metal derivatives are defined below.

### (Poly)amine Crosslinking Agents

According to a preferred embodiment, the crosslinking agent(s) are chosen from (poly)amine compounds.

The (poly)amine compound may be chosen in particular from polyamine compounds bearing several primary and/or secondary amine groups or from amino alkoxysilanes, and more particularly from amino alkoxysilane compounds, diamine compounds, triamine compounds, and mixtures thereof.

The amine compound may also be chosen from the compounds of formula (Z) below:

(NH₂)ᵣW′(OH)ₛ(SH)ₜ

in which:
s and t, which may be identical or different, represent an integer, it being understood that s and t cannot simultaneously be zero and that (r+s) or (r+t) is greater than or equal to 2;
W denotes a) a polymeric radical, preferably carbon-based or silicon-based, optionally interrupted with one or more heteroatoms or groups chosen from O, S, N, Si, C(X), and combinations thereof such as —O—, —O—C(X)—, —N(R)—C(X)—, —Si(R_{c})(R_{d})—O— with R representing a hydrogen atom or a (C₁-C₆)alkyl group such as methyl and X denoting 0 or S; and/or optionally substituted with one or more halogen atoms, or a group chosen from Rₐ(R_{b})N— and —(X′)ₐ—C(X)—(X″)_{b}—Rₐ;
or b) W denotes a linear or branched, saturated or unsaturated, or (hetero)cyclic, saturated or unsaturated, multivalent (at least divalent) group, in particular comprising between 1 and 500 carbon and/or silicon atoms, more particularly between 2 and 40 carbon and/or silicon atoms, even more particularly between 3 and 30 carbon and/or silicon atoms, preferably between 6 and 20 carbon atoms;
W being optionally interrupted with one or more heteroatoms or groups chosen from 0, S, N, Si, C(X), and combinations thereof such as —O—, —O—C(X)—, —N(R)—C(X)—, —Si(Rc)(Rd)-O— with R representing a hydrogen atom or a (C₁-C₆)alkyl group such as methyl; and/or
W being optionally substituted with one or more halogen atoms, or a group chosen from Ra(Rb)N— and —(X′)ₐ—C(X)—(X″)_{b}—Ra;
Rc and Rd, which may be identical or different, represent a (C₁-C₆)alkyl, aryl(C₁-C₄)alkyl or (C₁-C₆)alkoxy group.
X, X′ and X″, which may be identical or different, represent an oxygen or sulfur atom, or a group N(R_{b});
Rₐ and R_{b}, which may be identical or different, represent a hydrogen atom or a (C₁-C₆)alkyl or aryl(C₁-C₄)alkyl group such as benzyl; preferably, Rₐ and R_{b} represent a hydrogen atom; and
R_{c} and R_{d}, which may be identical or different, represent a (C₁-C₆)alkyl, aryl(C₁-C₄)alkyl or (C₁-C₁₀)alkoxy group.

As examples of compounds of formula (Z), mention may be made of 3-aminopropanol (list to be completed)

The (poly)amine compound may be a compound comprising from 2 to 20 carbon atoms, notably a non-polymeric compound.

The term “non-polymeric compound” means a compound which is not directly obtained via a monomer polymerization reaction.

(Poly)amine compounds that may be mentioned in particular include N-methyl-1,3-diaminopropane, N-propyl-1,3-diaminopropane, N-isopropyl-1,3-diaminopropane, N-cyclohexyl-1,3-diaminopropane, 2-(3-aminopropylamino)ethanol, 3-(2-aminoethyl)aminopropylamine, bis(3-aminopropyl)amine, methylbis(3-aminopropyl)amine, N-(3-aminopropyl)-1,4-diaminobutane, N,N-dimethyldipropylenetriamine, 1,2-bis(3-aminopropylamino)ethane, N,N′-bis(3-aminopropyl)-1,3-propanediamine, ethylenediamine, 1,3-propylenediamine, 1,4-butylenediamine, lysine, cystamine, xylenediamine, tris(2-aminoethyl)amine and spermidine.

The amine compound may also be chosen from amino alkoxysilanes, such as those of formula R′₁Si(OR′₂)_{z}(R′₃)ₓ in which:
R′₁ is a linear or branched, saturated or unsaturated, cyclic or acyclic C₁-C₆ hydrocarbon-based chain substituted with a group chosen from the amine groups NH₂ or NHR with R representing a C₁-C₄ alkyl, an aryl or an aryloxy substituted with an amino group or with a C₁-C₄ aminoalkyl group; R′₁ may be interrupted in its chain with a heteroatom (O, S, NH) or a carbonyl group (CO), R′₁ being linked to the silicon atom directly via a carbon atom,
R′₂ and R′₃, which may be identical or different, represent a linear or branched alkyl group comprising from 1 to 6 carbon atoms,
z denotes an integer ranging from 1 to 3, and
x denotes an integer ranging from 0 to 2, with z+x=3;

In particular, R′₁ is an acyclic chain. Preferably, R′₁ is a linear or branched, saturated or unsaturated C₁-C₆ hydrocarbon-based chain substituted with an amine NH₂ or NHR group, with R representing a C₁-C₆ alkyl group, C₃-C₆ cycloalkyl, or C₆ aromatic group. More preferentially, R′₁ is a saturated linear C₁-C₆ hydrocarbon-based chain substituted with an amine group NH₂. Even more preferentially, R′₁ is a saturated linear C₂-C₄ hydrocarbon-based chain substituted with an amine group NH₂.

In particular, R′₂ represents an alkyl group comprising from 1 to 4 carbon atoms; preferably, R′₂ represents a linear alkyl group comprising from 1 to 4 carbon atoms and more preferentially R′_{z} represents an ethyl group.

In particular, R′₃ represents an alkyl group comprising from 1 to 4 carbon atoms; preferably, R′₃ represents a linear alkyl group comprising from 1 to 4 carbon atoms and more preferentially R′₃ represents methyl or ethyl groups.

Preferably, z is equal to 3.

According to a particular embodiment, the amino alkoxysilane is chosen from 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane and N-(2-aminoethylaminomethyl)phenethyltrimethoxysilane.

Preferably, the amino alkoxysilane is chosen from 3-aminopropyltriethoxysilane (APTES), 3-aminoethyltriethoxysilane (AETES), 3-aminopropylmethyldiethoxysilane and N-(2-aminoethyl)-3-aminopropyltriethoxysilane, and more preferentially, the amino alkoxysilane is 3-aminopropyltriethoxysilane (APTES).

According to a preferred embodiment, the (poly)amine compound is chosen from 3-aminopropyltriethoxysilane (APTES), N-methyl-1,3-diaminopropane, N-propyl-1,3-diaminopropane, N-isopropyl-1,3-diaminopropane, N-cyclohexyl-1,3-diaminopropane, 2-(3-aminopropylamino)ethanol, 3-(2-aminoethyl)aminopropylamine, bis(3-aminopropyl)amine, methylbis(3-aminopropyl)amine, N-(3-aminopropyl)-1,4-diaminobutane, N,N-dimethyldipropylenetriamine, 1,2-bis(3-aminopropylamino)ethane, N,N′-bis(3-aminopropyl)-1,3-propanediamine, ethylenediamine, 1,3-propylenediamine, 1,4-butylenediamine and lysine.

Preferentially, the (poly)amine compound is chosen from ethylenediamine, 1,3-propylenediamine, 1,4-butylenediamine and 3-aminopropyltriethoxysilane (APTES), and more preferentially, the (poly)amine compound is ethylenediamine or 3-aminopropyltriethoxysilane (APTES).

The (poly)amine compound may also be chosen from amino polymers, notably having a weight-average molecular weight ranging from 500 g·mol⁻¹ to 1 000 000 g·mol⁻¹, preferably ranging from 500 g·mol⁻¹ to 500 000 g·mol⁻¹, and preferentially ranging from 500 g·mol⁻¹ to 100 000 g·mol⁻¹.

As amine polymers, mention may be made in particular of:
i) poly((C₂-C₅)alkyleneimines), and preferably polyethyleneimines and polypropyleneimines, notably poly(ethyleneimine), in particular the product sold under the reference 46,852-3 by the company Aldrich Chemical;
ii) poly(allylamine), in particular the product sold under the reference 47.913-6 by the company Aldrich Chemical:
iii) polyvinylamines and copolymers thereof, notably with vinylamides; in particular vinylamine/vinylformamide copolymers, such as those sold under the name Lupamin®9030 by the company BASF;
iv) polyamino acids containing NH₂ groups such as polylysine, in particular the product sold by the company JNC Corporation (formerly Chisso); v) aminodextran, in particular the product sold by the company CarboMer Inc.;
vi) amino polyvinyl alcohol, in particular the product sold by the company CarboMer Inc.; vii) copolymers based on acrylamidopropylamine;
viii) chitosans such as poly(D-glucosamine) sold under the reference Kionutrime CSG® by the company Kytozyme;
ix) polydimethylsiloxanes comprising primary amine groups at the end of the chain or on side chains, for example terminal or lateral aminopropyl groups, such as those chosen from (Ib), (IIb), (IIIb) and (III′b):

H₂N—[CH₂]₃—Si(R)₂—O—[Si(R)₂—O]ₙ—Si(R)₂—[CH₂]₃—NH₂ (Ib)

in which formula (IIb):
R, which may be identical or different, preferably identical, represents a (C₁-C₄)alkyl group such as methyl;
the value of n is such that the weight-average molecular weight of the polydimethylsiloxane ranges from 500 g·mol⁻¹ to 55 000 g·mol⁻¹.

(R)₃Si—O—[Si(R)₂—O]ₘ—[Si(R)(CH₂—CH₂—CH₂—NH₂)—O]ₙ—Si(R)₃ (IIb)

in which formula (IIb):
R, which may be identical or different, preferably identical, represents a (C₁-C₄)alkyl group such as methyl;
n and m are such that the weight-average molecular weight of the polydimethylsiloxane ranges from 1000 g·mol⁻¹ to 55 000 g·mol⁻¹.

H₂N—CH₂—CH₂—CH₂—Si(R)₂—O—[Si(R)₂—O]ₙ—Si(R)₂—R′ (IIIb)

in which formula (IIIb):
R, which may be identical or different, preferably identical, represents a (C₁-C₄)alkyl group such as methyl;
R′ represents a (C₂-C₆)alkyl group such as n-butyl, i-butyl or t-butyl, notably n-butyl;
the value of n is such that the weight-average molecular weight of the polydimethylsiloxane ranges from 500 to 3000 g·mol⁻¹.

As examples of compounds of formula (IIb), mention may be made of those sold under the names DMS-A11, DMS-A12, DMS-A15, DMS-A21, DMS-A31, DMS-A32 and DMS-A35 by the company Gelest.

As examples of polydimethylsiloxanes of formula (IIb), mention may be made of those sold under the names AMS-132, AMS-152, AMS-162, AMS-163, AMS-191 and AMS-1203 by the company Gelest.

As examples of polydimethylsiloxanes (IIIb), mention may be made of the products sold under the names MCR-A11 and MCR-A12 by the company Gelest. x) amine polymers, mention may also be made of the amodimethicones of formula (III′b):

R^{a}—Si(R)₂—[O—Si(R)₂]ₙ—[O—Si(R^{b})(A-NH—CH₂—CH₂—NH₂)ₘ—O—Si(R)₂—R^{c} (III′b)

in which formula (III′b):
R^{a}, R^{b} and R^{c}, which may be identical or different, represent a hydroxyl or C₁-C₄ alkyl group such as methyl;
R, which may be identical or different, preferably identical, represents a (C₁-C₄)alkyl group such as methyl;
A represents a (C₁-C₄)alkylene group, notably C₃, and m and n are such that the weight-average molecular mass of the compound of formula (III′b) ranges from 5000 g·mol⁻¹ to 500 000 g·mol⁻¹.

As amine polymers, mention may also be made of polyether amines, in particular those known under the reference Jeffamine from the company Hunstman; and notably the polyethylene glycol and/or polypropylene glycol α,ω-diamines (with an amine function at the end of the chain) such as those sold under the names Jeffamine D-230, D-400, D-2000, D-4000, ED-600, ED-9000, ED-2003.

As amine polymers, mention may also be made of α,ω-diamine polytetrahydrofuran (or polytetramethylene glycol), α,ω-diamine polybutadienes and amine-terminated polyamidoamine (PAMAM) dendrimers.

As amine polymers, mention may also be made of poly(meth)acrylates or poly(meth)acrylamides with primary or secondary side amine functions, such as poly(3-aminopropyl)methacrylamide, poly(2-aminoethyl)methacrylate.

As amine polymers, use is preferably made of chitosans, polydimethylsiloxanes comprising primary amine groups at the chain end or on side chains.

Preferably, the polyamine compounds are chosen from chitosans, polydimethylsiloxanes comprising primary amine groups at the chain end or on side chains, and APTES.

Thus, according to a preferred embodiment, the dispersion of the invention comprises a crosslinking agent chosen from (poly)amine compounds, in particular chosen from chitosans, aminoalkoxysilanes, polydimethylsiloxanes comprising primary amine groups at the end of the chain or on side chains, amodimethicones, polyglucosamines, and mixtures thereof.

More preferentially, the dispersion of the invention comprises a crosslinking agent chosen from chitosans, aminoalkoxysilanes and polydimethylsiloxanes comprising primary amine groups at the end of the chain or on side chains, and even more preferentially chosen from poly(D-glucosamine), 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane and polydimethylsiloxanes comprising terminal aminopropyl groups at the end of the chain, and even more preferentially 3-aminopropyltriethoxysilane (APTES).

(Poly)Thiolated and/or (Poly)Hydroxylated Crosslinking Agents

According to a preferred embodiment, the crosslinking agent is chosen from (poly)thiolated and/or (poly)hydroxylated compounds.

The (poly)thiolated and/or (poly)hydroxylated compound may in particular be organic or inorganic, preferably organic.

The (poly)thiolated and/or (poly)hydroxylated compound of the invention is chosen from liposoluble or non-liposoluble compounds; preferably, the (poly)thiolated and/or (poly)hydroxylated compound is liposoluble.

The term “liposoluble compound” means a compound that is soluble or miscible to at least 1% by weight in isododecane at 25° C.

In a preferred embodiment, the (poly)thiolated and/or (poly)hydroxylated compound is siliconized, i.e. it includes one or more hydroxyl groups, or one or more thiol groups or one or more hydroxyl groups and one or more thiol groups, and it also includes at least one siloxane chain.

In a particular embodiment, the (poly)thiolated and/or (poly)hydroxylated compound is inorganic. Mention may be made, for example, of polythiol silicones and polythiol silicas.

The (poly)thiolated and/or (poly)hydroxylated compound used in a composition according to the invention may in particular be chosen from non-polymeric (poly)thiolated and/or (poly)hydroxylated compounds.

For the purposes of the present invention, the term “non-polymeric compounds” means compounds which are not directly obtained via a monomer polymerization reaction.

According to one embodiment of the invention, the (poly)thiolated and/or (poly)hydroxylated compound is organic, non-polymeric and of formula (IVb) below and also the solvates thereof such as hydrates:

(HO)ₚL(SH)_{q} (IVb)

in which formula (IVb):
p and q, which may be identical or different, represent an integer, it being understood that the sum p+q is greater than or equal to 2; preferably the sum p+q is between 2 and 10, preferably between 2 and 5 inclusive;
L denotes a saturated or unsaturated linear or branched, or saturated or unsaturated (hetero)cyclic, multivalent (at least divalent) group, in particular comprising between 1 and 500 carbon and/or silicon atoms, more particularly between 2 and 40 carbon and/or silicon atoms, even more particularly between 3 and 30 carbon and/or silicon atoms, preferably between 6 and 20 carbon atoms;
L being optionally interrupted and/or terminated with one or more heteroatoms or groups chosen from O, S, N, Si, C(X), and combinations thereof such as —O—, —O—C(X)—, —N(R)—C(X)—, —Si(Rc)(Rd)-O— with R representing a hydrogen atom or a (C₁-C₆)alkyl group such as methyl; and/or
L being optionally substituted with one or more halogen atoms, or a group chosen from Rₐ(Rb)N— and —(X′)a-C(X)—(X″)_{b}—Ra;
X, X′ and X″, which may be identical or different, represent an oxygen or sulfur atom, or a group N(Rb);
a and b are equal to 0 or 1; preferably, the sum of a+b is 1;
Ra and Rb, which may be identical or different, represent a hydrogen atom or a (C₁-C₆)alkyl or aryl(C₁-C₄)alkyl group such as benzyl; preferably, Ra and Rb represent a hydrogen atom;
Rc and Rd, which may be identical or different, represent a (C₁-C₆)alkyl, aryl(C₁-C₄)alkyl or (C₁-C₆)alkoxy group.

According to one embodiment of the invention, in the above formula (IVb), q is equal to 0 and p is an integer greater than or equal to 2, preferably p is an integer from 2 to 10, preferably from 2 to 5.

According to a particular embodiment of the invention, the (poly)thiolated and/or (poly)hydroxylated compound is chosen from polyhydroxylated compounds, in particular polyhydroxylated compounds comprising from 2 to 20 carbon atoms, in particular non-polymeric polyhydroxylated compound(s).

The (poly)thiolated and/or (poly)hydroxylated compound used according to the invention may be chosen from hydroxyalkoxysiloxane or thioalkoxysiloxane compounds, it being understood that these compounds may also comprise one or more primary or secondary amine groups.

A polyhydroxylated compound used according to the invention is an organic compound comprising at least two hydroxyl functions. This compound may comprise other unreactive chemical functions such as ester, amide, ketone or urethane functions. It is possible to use a mixture of different polyhydroxylated compounds.

According to another variant, the polyhydroxylated compound used according to the invention is an inorganic compound comprising at least two hydroxyl functions. This compound may comprise other unreactive chemical functions such as ester, amide, ketone or urethane functions. It is possible to use a mixture of different polyhydroxylated compounds such as a mixture of organic and mineral polyhydroxylated compounds.

According to another embodiment, the thiolated and/or hydroxylated compound is chosen from polyhydroxylated, polythiolated, and polymeric polyhydroxylated and polythiolated compounds.

According to one embodiment of the invention, the polyhydroxylated compound is a non-polymeric organic compound of formula (Vb) below:

L(OH)ₚ (Vb)

in which formula (Vb):
p denotes an integer greater than or equal to 2, preferably between 2 and 10, preferably between 2 and 5 inclusive;
L is as defined previously; preferably, L denotes a saturated or unsaturated linear or branched, or saturated or unsaturated (hetero)cyclic, multivalent (at least divalent) radical comprising between 8 and 30 carbon and/or silicon atoms, preferably between 10 and 20 carbon and/or silicon atoms, L also possibly being interrupted with one or more oxygen atoms, and/or comprising one or more functions chosen from amino, ether, thio ether, ester, thio ester, ketone, thio ketone, amide and thio amide functions.

The polyhydroxylated compound is preferably a diol compound.

Preferably, L denotes a C₈-C₁₈ multivalent radical, which is notably linear.

Preferentially, the polyhydroxylated compound is a liposoluble polyol, in particular a C₈-C₁₈ diol, which is notably linear. Advantageously, the C₈-C₁₈ chain is a hydrocarbon-based chain, i.e. formed from carbon and hydrogen.

In particular, the liposoluble polyol is a linear C₈-C₁₆ and notably C₁₀-C₁₄ diol.

As polyol of formula (Vb), mention may be made of 1,8-octanediol, 1,10-decanediol, 1,12-dodecanediol, 1,14-tetradecanediol, 1,16-hexadecanediol and 1,18-octadecanediol.

Use is preferably made of 1,10-decanediol, 1,12-dodecanediol or 1,14-tetradecanediol. 1,12-Dodecanediol is preferentially used.

According to a particular embodiment of the invention, the thiolated and/or hydroxylated compound is chosen from polythiolated compounds also known as “polymercapto” compounds.

The polythiolated compounds used according to the invention may be water-soluble or liposoluble, and are preferably liposoluble.

According to a particular embodiment of the invention, the thiolated and/or hydroxylated compound is chosen from polythiolated compounds, notably polythiolated compounds comprising from 2 to 20 carbon atoms.

According to a preferred embodiment, the thiolated and/or hydroxylated compound is thiolated, non-polymeric and of formula (Vb) defined above, in which p is 0 and q is an integer greater than or equal to 2, preferably p is an integer between 2 and 10, preferably between 2 and 5 inclusive.

In particular, a polythiolated compound that is suitable for use in the invention is a non-polymeric organic compound of formula (VIb) below: L(SH)q (VIb)

in which formula (VIb) L is as defined in formula (Vb) above and q represents an integer greater than or equal to 2, preferably between 2 and 10, preferably between 2 and 5 inclusive.

The polythiolated compounds that are suitable for use in the invention are preferably dithiol compounds.

Preferably, L denotes a C₈-C₁₈ multivalent radical, which is notably linear. Preferentially, the liposoluble polythiol is a notably linear C₈-C₁₈ dithiol. Advantageously, the C₈-C₁₈ chain is a hydrocarbon-based chain, i.e. formed from carbon and hydrogen. In particular, the liposoluble polythiol is a linear C₈-C₁₆ and notably C₁₀-C₁₄ dithiol.

As polyol of formula (VIb), mention may be made of 1,8-octanedithiol, 1,10-decanedithiol, 1,12-dodecanedithiol, 1,14-tetradecanedithiol, 1,16-hexadecanedithiol and 1,18-octadecanedithiol.

Use is preferably made of 1,10-decanedithiol, 1,12-dodecanedithiol or 1,14-tetradecanedithiol. 1,12-Dodecanedithiol is preferentially used.

According to another embodiment of the invention, the thiolated and/or hydroxylated compound is thiolated and hydroxylated, non-polymeric and of formula (VIb) as defined above, in which q and p are integers greater than or equal to 1; preferably, the sum p+q is an integer of from 2 to 10, preferably from 2 to 5, and preferably q>p.

According to yet another particular embodiment of the invention, the thiolated and/or hydroxylated compound is chosen from (poly)hydroxylated and (poly)thiolated compounds, notably (poly)hydroxylated and (poly)thiolated compounds comprising from 2 to 20 carbon atoms, and notably non-polymeric (poly)hydroxylated and (poly)thiolated compounds.

According to another particular embodiment of the invention, the thiolated and/or hydroxylated compound is chosen from hydroxylated and/or thiolated alkoxysiloxanes, such as those of formula (VIIb) below: R′₁—Si(OR′₂)_{z}(R′₃)ₓ (VIIb) in which formula (VIIb):
R′₁ is a linear or branched, saturated or unsaturated, cyclic or acyclic C₁-C₁₂ hydrocarbon-based chain substituted with one or more groups chosen from the following groups:
hydroxyl or thiol, preferably thiol,
aryl, aryloxy, arylthio or arylamino, the aryl group being substituted with one or more hydroxyl, thiol, hydroxy(C₁-C₆)alkyl or thio(C₁-C₆)alkyl groups, preferably thio(C₁-C₆)alkyl, and
R′₁ is optionally interrupted in its hydrocarbon-based chain with one or more heteroatoms such as O, S, N, a carbonyl group (CO), or a combination thereof such as ester —C(O)—O—, or amide —C(O)—N(H)—, R′₁ being bonded to the silicon atom directly via a carbon atom,
R′₂ and R′₃, which may be identical or different, represent a linear or branched alkyl group comprising from 1 to 6 carbon atoms, preferably from 1 to 4 carbon atom(s) such as methyl,
z denotes an integer ranging from 1 to 3, and
x denotes an integer ranging from 0 to 2, with z+x=3.

In particular, R′₂ represents an alkyl group comprising from 1 to 4 carbon atoms. Preferably, R′₂ represents a linear alkyl group comprising from 1 to 4 carbon atoms. More preferentially, R′₂ represents an ethyl group.

In particular, R′₃ represents an alkyl group comprising from 1 to 4 carbon atoms. Preferably, R′₃ represents a linear alkyl group comprising from 1 to 4 carbon atoms. More preferentially, R′₃ represents a methyl or ethyl group.

In particular, R′₁ is an acyclic chain. More particularly, R′₁ is a linear or branched, saturated or unsaturated C₁-C₆ hydrocarbon-based chain, substituted with one or more hydroxyl or thiol groups, preferably thiol. Preferentially, R′₁ is a saturated linear C₁-C₆ hydrocarbon-based chain substituted with a hydroxyl or thiol group, preferably thiol.

More preferentially, R′₁ is a saturated linear C₂-C₄ hydrocarbon-based chain substituted with a hydroxyl or thiol group, preferably thiol.

Preferably, R′₁ is a saturated linear C₁-C₆ hydrocarbon-based chain substituted with a hydroxyl or thiol group, preferably thiol, R′₂ represents an alkyl group comprising from 1 to 4 carbon atoms,

R′₃ represents an alkyl group comprising from 1 to 4 carbon atoms.

Preferably, z is equal to 3.

According to an even more particular embodiment of the invention, the alkoxysiloxanes (VIIb) are chosen from those of formula (VII′b) below:

(R¹O)(R²)(R³)Si—CH₂—[N(R⁴)-L¹]ₚ-X—H (VII′b)

in which formula (VII′b):
p is 0 or 1;
X represents an oxygen or sulfur atom, preferably a sulfur atom;
R¹ represents a (C₁-C₆)alkyl radical;
R₂ and R₃, which may be identical or different, preferably identical, are chosen from:
   a (C₁-C₆)alkoxy group, in particular C₁-C₄;
   a (C₁-C₆)alkyl group;
R⁴ represents a hydrogen atom, or a (C₁-C₆)alkyl group such as methyl;
L¹ represents a divalent, saturated, linear or branched C₁-C₂₀ hydrocarbon-based radical.

According to an even more particular embodiment of the invention, the alkoxysiloxane compounds of formula (VII′b) are chosen from the compounds of formula (VII″b) below:

(R′¹O)(R′²)(R′³)Si—CH(R⁴)—CH(R⁵)-(L²)_{q}-X—H (VII″b)

in which formula (VII″b):
q is 0 or 1;
X represents an oxygen or sulfur atom, preferably a sulfur atom;
R′¹ denotes a (C₁-C₆)alkyl radical;
R′₂ and R′₃, which may be identical or different, preferably identical, are chosen from:
a (C₁-C₆)alkoxy group, in particular C₁-C₄;
a (C₁-C₆)alkyl radical;
R⁵ represents a hydrogen atom or a C₁-C₄ alkyl group optionally substituted with an amino, thiol or hydroxyl group;
R⁴ represents a hydrogen atom or a C₁-C₄ alkyl group, in particular methyl;
L² represents a divalent, linear or branched, saturated C₁-C₂₀ hydrocarbon-based group, optionally interrupted with a heteroatom such as —NH—, optionally substituted with one or more hydroxyl, thiol or amino groups.

Preferably, the alkoxysilane of formula (VIIb) is chosen from 4-(trimethoxysilyl)-1-butanol, 3-(trimethoxysilyl)-1-propanol, 3-(triethoxysilyl)-1-propanol, 11-(trimethoxysilyl)-1-undecanethiol, 4-(trimethoxysilyl)-2-butanethiol, 2-(triethoxysilyl)ethanethiol, 3-(triethoxysilyl)-1-propanethiol, 2-(trimethoxysilyl)ethanethiol, 3-(trimethoxysilyl)-1-propanethiol and 3-(dimethoxymethylsilyl)-1-propanethiol.

More preferentially, the alkoxysilane of formula (VIIb) is chosen from 2-(triethoxysilyl)ethanethiol (18236-15-2) and 3-(triethoxysilyl)-1-propanethiol (14814-09-6).

According to another embodiment, the thiol compound is a particle bearing at least two thiol groups. It is, for example, a silica functionalized with radicals, notably of the alkyl type, substituted with thiol functions.

According to this embodiment, the thiol compound may refer to the particles sold under the name SiliaMetS® Thiol by the company Silicycle or under the name SP-THIO-SILICA by the company Suprasciences.

The thiol compound may be chosen from the polythiol silicas prepared according to the procedure described in J. Mater. Chem., 2007, 17, 3726-3732; J. Am. Chem. Soc., 2005, 127 (23), pp 8492-8498; 10.1109/ICBBE.2010.5517542; Chemical Engineering Journal, 2011, 171 (3), 1004-1011; Minerals Engineering, 2012, 35, 20-26; Colloids and Surfaces A: Physicochemical and Engineering Aspects, 380, (1-3), 229-233), Advanced Science, Engineering and Medicine, Volume 5, Number 9, September 2013, pages 984-987(4).

The particle bearing at least two thiol groups may also be obtained by reacting a silica particle such as the Sunsphere® particles sold by Asahi Glass with mercaptoalkylalkoxysilanes such as 3-mercaptopropyltriethoxysilane.

Preferably, the thiol compound is a particle bearing at least two thiol groups obtained by reacting a Sunsphere® silica particle sold by Asahi Glass, preferably a 5 μm Sunsphere particle, with 3-mercaptopropyltriethoxysilane according to the publication, Journal of Chromatography A, Volume 1217, Issue 47, 19-11-2010, pages 7448-7454.

According to a second embodiment, the polythiolated particles may be present in the form of latices bearing thiol groups such as those described in Colloids and Surfaces A: Physiochemical and Engineering Aspects 153, 1999, 421-427.

According to another embodiment, the thiol compound is a particle coated with compound(s) bearing at least two thiol functions.

The (poly)thiolated and/or (poly)hydroxylated compound used in a composition according to the invention may in particular be chosen from polymeric (poly)thiolated and/or (poly)hydroxylated compounds.

The polymeric (poly)thiolated and/or (poly)hydroxylated compounds may be homopolymers, copolymers, star, comb, brush and dendritic compounds bearing hydroxyl and/or thiol units. The polymers may be of natural origin such as polysaccharides or polypeptides, or of synthetic origin such as acrylic polymers, polyesters or polyglycols. The hydroxyl and thiol units may be present as terminal or side groups.

Examples that may be mentioned include the polymers described in the following scientific articles, patent applications and patents: Polymers containing groups of biological activity, C. G. Overberger et al., Polytechnic Institute of Brooklyn, http://pac.iupac.org/publications/pac/pdf/l962/pdf/0402x0521.pdf; EP 1 247 515 A2; U.S. Pat. No. 3,676,440; and EP 1 572 778.

The thiolated and/or hydroxylated polymers that may be suitable for use in the invention are preferably organic or silicone-based, more preferentially of formula (VIIIb):

(HO)ₚPOLY(SH)_{q} (VIIIb)

in which formula (VIIIb):
p and q, which may be identical or different, represent an integer, it being understood that the sum p+q is greater than or equal to 3;
POLY denotes a polymeric radical which is preferably carbon-based or silicon-based;
POLY being optionally interrupted with one or more heteroatoms or groups chosen from O, S, N, Si and C(X), and combinations thereof such as —O—, —O—C(X)—, —N(R)—C(X)— or —Si(R_{c})(R_{d})—O— with R representing a hydrogen atom or a (C₁-C₆)alkyl group such as methyl; and/or
   POLY being optionally substituted with one or more halogen atoms, or a group chosen from Rₐ(R_{b})N— and —(X′)ₐ—C(X)—(X″)_{b}—Rₐ;
X, X′ and X″, which may be identical or different, represent an oxygen or sulfur atom, or a group N(R_{b});
a and b being equal to 0 or 1; preferably, the sum a+b is equal to 1;
Rₐ and R_{b}, which may be identical or different, represent a hydrogen atom or a (C₁-C₁₀)alkyl or aryl(C₁-C₄)alkyl group such as benzyl; preferably, Rₐ and R_{b} represent a hydrogen atom; and
R_{c} and R_{d}, which may be identical or different, represent a (C₁-C₁₀)alkyl, aryl(C₁-C₄)alkyl or (C₁-C₁₀)alkoxy group.

According to a particular embodiment, the thiolated and/or hydroxylated compound is chosen from (poly)hydroxylated polymers, also known as “polyol”.

According to another embodiment of the invention, the (poly)hydroxylated polymer(s) are chosen from those of formula (VIII′b) below:

POLY(OH)ₚ (VIII′b)

in which formula (VIII′b):
p denotes an integer greater than or equal to 2, and
POLY denotes a carbon-based or silicone-based polymeric radical, POLY also possibly containing one or more heteroatoms such as O, N or S, and/or one or more functions chosen from amino, (thio)-ester, (thio)-ketone, (thio)-amide, (thio)-urea and (thio)carbamate functions, and/or possibly being substituted with one or more linear or branched (C₁-C₁₀)alkyl or linear or branched (C₁-C₁₀)alkoxy groups, it being understood that when POLY is substituted, the hydroxyl groups may be borne by the substituent(s).

The weight-average molecular weight of the polyol polymer compounds, such as those of formula (VIII′b), is generally between 500 and 400 000 g·mol⁻¹, preferably between 500 and 150 000 g·mol⁻¹.

Preferably, the polyhydroxylated polymers (VIII′b) may be (di)ol polymers, notably polyolefin (poly)ols, polydi(C₁-C₆)alkylsiloxane (poly)ols or polyester (poly)ols; more preferentially, the (poly)ols are diols.

The polyolefin (poly)ols may be polydienes bearing hydroxyl end groups, for instance those described in FR-A-2 782 723. They may be chosen from (poly)ols derived from homopolymers and copolymers of polybutadiene, of polyisoprene and of poly(1,3-pentadiene). They preferably have a number-average molecular mass (Mn) of less than 7000 g·mol⁻¹, preferably between 1000 and 5000 g·mol⁻¹. Mention will be made in particular of the hydroxylated polybutadienes sold by the company Cray Valley under the brand names Poly BD R45HTLO, Poly BD R45V and Poly BD R-20 LM, which will preferably be used hydrogenated; and also (poly)hydroxylated hydrogenated (1,2-polybutadienes), such as G13000 of Mn=3100, G12000 (Mn=2100) and G11000 (Mn=1500) sold by the company Nisso.

More particularly, the compounds of formula (VIII′b) are chosen from the polyolefin (poly)ols of formula (IXb) below:

HO-[ALK⁴]ₘ-[ALK⁵]ₙ—XH (IXb)

in which formula (IXb):
ALK⁴ and ALK⁵, which may be identical or different, preferably different, represent a linear or branched (C₁-C₆)alkylene group, optionally substituted with one or more hydroxyl, thiol or amino groups, preferably ALK⁴ represents a linear (C₁-C₆)alkylene group such as n-butylene, and ALK⁵ represents a branched (C₃-C₆)alkylene group such as i-butylene;
X represents an oxygen or sulfur atom or a group N(Ra) with Ra representing a hydrogen atom or a (C₁-C₄)alkyl group; preferably, X represents a hydroxyl or thiol group, more preferably hydroxyl; and
n and m, which may be identical or different, represent an integer, with n+m representing an integer greater than or equal to 1.

The (poly)ols of formula (IXb) may in particular be chosen from polyolefins bearing hydroxyl end groups.

Among the polyolefins bearing hydroxyl end groups, mention may be made preferentially of polyolefin homopolymers or copolymers bearing α,ω-hydroxy end groups, such as polyisobutylenes bearing α,ω-hydroxy end groups and the copolymers of formula (IX′b):

HO—[(CH₂)₄]ₘ—[CH₂—CH(R_{d})]ₙ—OH (IX′b)

in which formula (IX′) R_{d} represents a hydrogen atom, a (C₁-C₄)alkyl group, preferably (C₁-C₄) such as ethyl, and m and n are as defined previously. The (poly)ols of the invention are notably those sold by Mitsubishi under the brand name Polytail. Hydrogenated polybutadiene diols are preferably used.

The (poly)ols of formula (IXb) may in particular be chosen from polydialkylsiloxane (di)ols, particularly from those of formula (Xb) below:

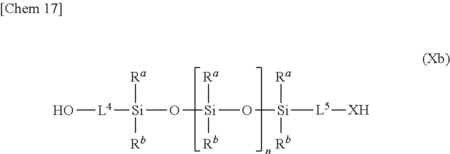

in which formula (Xb):
R^{a} and R^{b}, which may be identical or different, preferably identical, represent a group from among: (C₁-C₆)alkyl optionally substituted with one or more hydroxyl, amino or thiol groups; (C₁-C₆)alkoxy such as methoxy; aryl such as phenyl; aryloxy such as phenoxy; aryl(C₁-C₄)alkyl such as benzyl; or aryl(C₁-C₄)alkoxy such as benzoxy; preferably (C₁-C₄)alkyl such as methyl;
n represents an integer greater than or equal to 1 and more particularly the value of n is such that the weight-average molecular weight of the silicone is between 500 and 55 000 g·mol⁻¹; in particular, n is an integer from 1 to 100, preferably from 5 to 50 and preferentially from 10 to 30; and
L⁴ and L⁵, which may be identical or different, represent a covalent bond or a saturated or unsaturated, linear or branched, optionally cyclic hydrocarbon-based chain comprising from 1 to 100 carbon atoms, optionally interrupted with one or more heteroatoms such as oxygen, sulfur or nitrogen, in particular oxygen, more preferentially a (C₁-C₆)alkylene, (C₁-C₆)alkylenoxy, oxy(C₁-C₆)alkylene, (C₁-C₆)alkylenoxy(C₁-C₆)alkylene, (C₁-C₆)alkylenoxy(C₁-C₆)alkylenoxy or oxy(C₁-C₆)alkylenoxy(C₁-C₆)alkylene group;
X represents an oxygen or sulfur atom, preferably oxygen.

Preferentially, the polydialkylsiloxane (di)ols of formula (Xb) are chosen from polydimethylsiloxanes, notably the polydiols of formula (XIb) below:

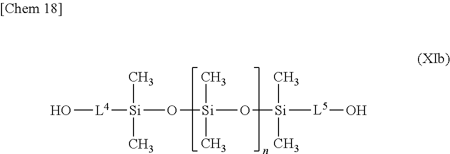

in which formula (XIb):
L⁴ and L⁵ are as defined previously, and preferably represent a divalent group chosen from —R₂—, —O—R₂—, —R₂—O— and —R₂—O—R′₂—, preferably —R₂—O—R′₂—, with R₂ and R′₂, which may be identical or different, representing a linear or branched (C₂-C₆)alkylene group, such as ethylene or propylene; and
n represents an integer between 1 and 100 inclusive, preferably between 5 and 50 and preferentially between 10 and 30.

Polydimethylsiloxanes diols that may be used include those sold under the names KF-6000, KF-6001, KF-6002 and KF-6003 by the company Shin-Etsu Chemicals. Use is preferably made of the polydimethylsiloxane diol of formula (XIIb) below:

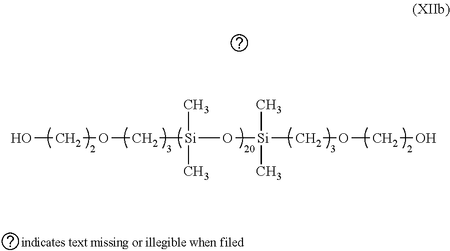

Use may also be made of dimethiconols, which are polydimethylsiloxanes bearing OH terminal functions. Mention may be made, for example, of the product sold under the name Xiameter PMX-1502 Fluid by the company Dow Corning.

According to a particular form of the invention, the (poly)ols of formula (XIIb) are chosen from the polyhydroxylated compounds of formula (XIIIb) below:

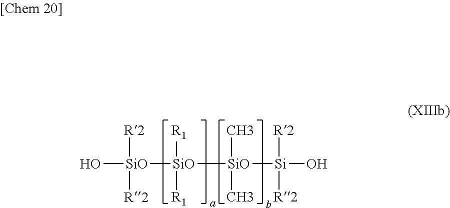

in which formula (XIIIb):
R₁, which may be identical or different, independently represents a hydroxyl group; an alkyl group containing from 1 to 10 carbon atoms, preferably 1 to 4 carbon atoms, notably 1 to 2 carbon atoms such as a methyl; an alkoxy group containing from 1 to 2 carbon atoms; or a group —(CH₂)ₛ—Si(R₄)₃ in which s denotes an integer ranging from 1 to 4 such as 2 and R₄ independently denotes an alkoxy radical containing from 1 to 2 carbon atoms;
R′₂ and R″₂ independently represent an alkyl group containing from 1 to 10 carbon atoms, preferably from 1 to 4 carbon atoms, notably 1 to 2 carbon atoms such as a methyl;
a denotes an integer ranging from 0 to 10, b denotes an integer ranging from 0 to 500 with a+b≥4.

Among the compounds of formula (XIIIb), mention may be made of polydimethylsiloxanes (PDMS) bearing hydroxyl terminal functions, such as the compounds sold by the company Shin-Etsu under the name KF-9701 or X-21-5841, or those sold by the company Sigma-Aldrich under the reference 481939 (Mn˜550 g·mol⁻¹, ˜25 cSt), 481955 (˜65 cSt) or 481963 (˜750 cSt). Mention may also be made of the compounds sold by the company Gelest under the name DMS-S12 (16-32 cSt), DMS-S15 (45-85 cSt), DMS-S21 (90-120 cSt), DMS-S27 (700-800 cSt) or DMS-S31 (˜1000 cSt).

According to a preferred embodiment, the silicone(s) of formula (XIIIb) used in the context of the invention are chosen from the compounds of formula (XIIIb) in which:
R₁ independently represents an alkyl group containing from 1 to 10 carbon atoms, preferably from 1 to 4 carbon atoms and more particularly from 1 to 2 carbon atoms, such as a methyl;
R′₂ and R″₂ independently represent an alkyl group containing from 1 to 10 carbon atoms, preferably an alkyl group containing from 1 to 4 carbon atoms and more particularly from 1 to 2 carbon atoms such as methyl;
b denotes an integer ranging from 0 to 10, a denotes an integer ranging from 0 to 5 with a+b≥4.

According to another embodiment of the invention, the polythiol compound is a thiolated polymer compound.

The methods for preparing the thiolated polymers used according to the invention are known to those skilled in the art; several methods are reported hereinbelow in a non-limiting manner.

The thiolated polymers used according to the invention may be obtained by polymerization or polycondensation of monomer units bearing thiol or protected thiol functions, optionally as a copolymerization or co-polycondensation of monomer units free of thiol or protected thiol functions. Alternatively, the thiolated polymers used according to the invention may be obtained by addition of hydrogen sulfide, of salts thereof such as sodium hydrogen sulfide or potassium sulfide or alternatively a group that is capable of forming a carbon-sulfur bond such as thiourea derivatives or thiosulfate, on a polymer bearing at least one double bond. The thiolated polymers used according to the invention may also be obtained by nucleophilic substitution of a leaving group present on a polymer chain (for example a halogen such as chlorine or bromine, or a sulfonic ester such as mesylate or tosylate) with a compound including at least one sulfur atom such as those mentioned previously.

The thiolated polymers used according to the invention may also be obtained by reaction of polymers including nucleophilic groups such as amines on electrophilic compounds including a sulfur atom, such as 2-oxo-4-thiazolidinecarboxylic acid, also known as procysteine:

TABLE 2

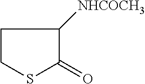

N-acetyl homocysteine thiolactone

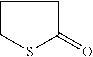

γ-thiobutyrolactone

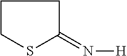

iminothiolane

According to one embodiment of the invention, the thiolated polymers used according to the invention are polymers which are soluble in cosmetic media, particularly in aqueous or aqueous-alcoholic media. They are more preferentially obtained from amino polymers and their ammonium salts or from polyhydroxylated polymers.

According to another embodiment of the invention, the thiolated polymers used according to the invention are polymers that are soluble in lipophilic media.

According to one embodiment of the invention, the polythiol compound is a polymeric compound of formula (XIVb) below:

POLY(SH)_{q} (XIVb)

in which formula (XIVb):
q denotes an integer greater than or equal to 2, and
POLY denotes a carbon-based and/or silicone-based, preferably silicone-based, polymeric radical, POLY also possibly containing one or more heteroatoms such as O, N or S, and/or one or more functions chosen from (thio)ester, (thio)ketone, (thio)amide, (thio)urea and (thio)carbamate functions, and/or possibly being substituted with one or more linear or branched (C₁-C₁₀)alkyl or linear or branched (C₁-C₁₀)alkoxy groups, it being understood that when POLY is substituted, the thiol functions may be borne by the substituent(s).

The weight-average molecular weight of the polythiol polymer compounds, such as those of formula (XIVb), is generally between 500 and 400 000 g·mol⁻¹, preferably between 500 and 150 000 g·mol⁻¹.

According to a particular embodiment of the invention, the polythiolated compounds are chosen from the polyorganosiloxanes of formula (XIV′b) below:

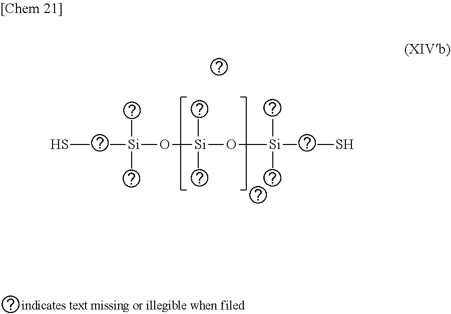

in which formula (XIV′b):
R^{a} and R^{b}, which may be identical or different, preferably identical, represent a group from among: (C₁-C₄)alkyl such as methyl, (C₁-C₄)alkoxy such as methoxy, aryl such as phenyl, aryloxy such as phenoxy, aryl(C₁-C₄)alkyl such as benzyl, or aryl(C₁-C₄)alkoxy such as benzoxy, preferably (C₁-C₄)alkyl such as methyl,
n represents an integer greater than or equal to 1 and more particularly the value of n is such that the weight-average molecular weight of the silicone ranges from 500 to 55 000 g·mol⁻¹; in particular, n is an integer ranging from 1 to 100, preferably ranging from 5 to 50 and preferentially ranging from 10 to 30; and
L⁴ and L⁵ are as defined above in formula (XII), in particular represent a covalent bond or a (C₁-C₆)alkylene, (C₁-C₆)alkylene-oxy, oxy-(C₁-C₆)alkylene, (C₁-C₆)alkylene-oxy(C₁-C₆)alkylene, (C₁-C₆)alkylene-oxy(C₁-C₆)alkylenoxy or oxy(C₁-C₆)alkylene-oxy(C₁-C₆)alkylene group, preferably a (C₁-C₆)alkylene, (C₁-C₆)alkylene-oxy, oxy-(C₁-C₆)alkylene or (C₁-C₆)alkylene-oxy(C₁-C₆)alkylene group.

Preferentially, the polydimethylsiloxane thiols are chosen from those of formula (XIV″b):

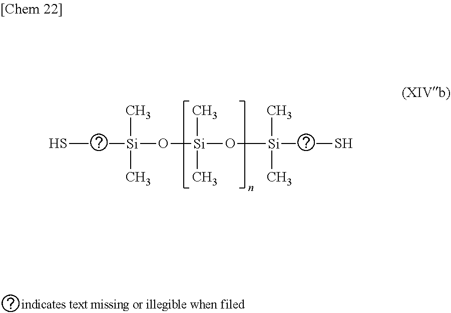

in which formula (XIV″b):
L⁴ and L⁵ represent a linear or branched, optionally cyclic, saturated or unsaturated hydrocarbon-based chain comprising from 1 to 100 carbon atoms, optionally interrupted with one or more heteroatoms such as oxygen, sulfur or nitrogen, in particular oxygen; preferably L⁴ and L⁵ represent a (C₁-C₆)alkylene, (C₁-C₆)alkylenoxy, oxy(C₁-C₆)alkylene or (C₁-C₆)alkylenoxy(C₁-C₆)alkylene group, more preferentially a divalent group chosen from —R₂—, —O—R₂—, —R₂—O— and —R₂—O—R₂—, preferably —R₂—O—R₂—, with R₂ representing a linear or branched, preferably linear, (C₂-C₆)alkylene group, such as ethylene or n-propylene;
n represents an integer from 1 to 100, preferably from 5 to 50, and preferentially from 10 to 30.

As thiolated poly(C₁-C₄)alkylsiloxanes, mention may be made of mercaptosiloxanes or thiolated siloxanes in which the thiol functions are at the chain ends, sold by the company Shin-Etsu under the reference X-22-167B, and mercaptosiloxane in which the mercapto functions are pendent, sold by the company Shin-Etsu under the reference KF-2001, or polydimethylsiloxanes in which the thiol functions are at the chain ends, via thio-n-propyl, 80-120 groups, sold by the company Gelest under the name DMS-SM 21, of formula (XIV′″b):

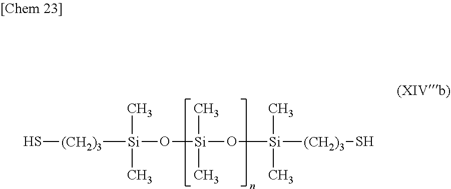

Preferentially, the polythiolated compounds are chosen from those of formula (XVb) below:

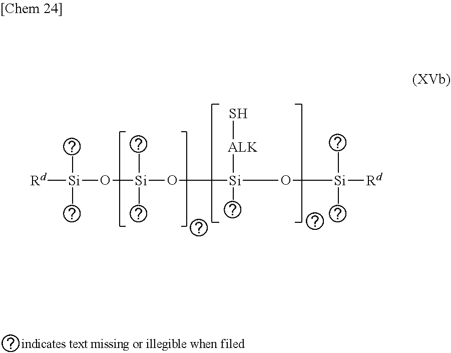

in which formula (XVb):
R^{a}, R^{b} and R^{d}, which may be identical or different, preferably identical, represent a group from among: (C₁-C₆)alkyl optionally substituted with a hydroxyl or amino group, preferably (C₁-C₄)alkyl such as methyl; (C₁-C₄)alkoxy such as methoxy; aryl such as phenyl; aryloxy such as phenoxy; aryl(C₁-C₄)alkyl such as benzyl; or aryl(C₁-C₄)alkoxy such as benzoxy; preferably (C₁-C₄)alkyl such as methyl;
   R^{d} may also represent a (C₁-C₆)alkyl group substituted with a (C₁-C₄)alkylamino or amino or thiol group, preferably (C₁-C₄)alkyl such as methyl;
ALK represents a linear or branched, optionally cyclic, saturated or unsaturated hydrocarbon-based chain comprising from 1 to 100 carbon atoms, optionally interrupted with one or more heteroatoms such as oxygen, sulfur or nitrogen (in particular O), a (thio)carbonyl group C(X) with X representing O or S, or combinations thereof such as —O—, —O—C(O)— or —C(O)—O—; preferably, ALK represents a (C₁-C₆)alkylene and more preferentially (C₁-C₄)alkylene group such as propylene;
n and m, which may be identical or different, representing an integer greater than 2 and more particularly the values of m and n are such that the weight-average molecular weight of the silicone is between 1000 and 55 000 g·mol⁻¹.

Preferentially, the polydi(C₁-C₄)alkylsiloxanes of formula (XVb) are of formula (XV′b) below:

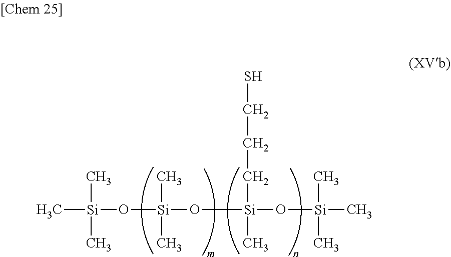

in which formula (XV′b) the values of n and m are such that the weight-average molecular weight of the silicone is between 1000 and 55 000 g·mol⁻¹.

As examples of silicones of formula (XV′), mention may be made of those sold by the company Genesee Polymers under the names GP-367, GP-71-SS, GP-800 and GP-710. Preferably, the silicone of formula (XV′) is the compound GP-367 sold by the company Genesee Polymers.

The polythiol silicones are notably polydimethylsiloxanes containing at least two thiol groups, for instance the products SMS-022, SMS-042 and SMS-992 sold by the company Gelest in https://www.gpcsilicones.com/products/silicone-fluids/mercapto-functional, https://www.shinetsusilicone-global.com/products/type/oil/detail/search/deg07.shtml, and 1053_Reactive Silicones_Silanes/Silicones-Gelest.

According to another embodiment, the thiolated compound is a particle bearing at least two thiol groups, such as a silica functionalized with radicals, for example of the alkyl type, substituted with thiol functions, or a particle covered with compound(s) bearing at least two thiol functions (coating), said particle possibly being a sphere, a fibre, a rod or an amorphous structure. According to a particular embodiment of the invention, the hydroxylated and/or thiolated compound is chosen from polymeric compounds such as hyperbranched polymers and dendrimers.

“Hyperbranched polymers” are molecular constructions having a branched structure, generally around a core. Their structure is generally free of symmetry. Specifically, the base units or monomers which served for the construction of the hyperbranched polymer may be of different nature and their distribution is irregular. The branches of the polymer may be of different nature and lengths. The number of base units, or monomers, may be different according to the different branchings. While being asymmetric, hyperbranched polymers may have an extremely branched structure, around a core; successive generations or layers of branching; a layer of terminal chains.

Hyperbranched polymers are generally derived from the polycondensation of one or more monomers ABx, A and B being reactive groups that are capable of reacting together, x being an integer greater than or equal to 2, but other preparation processes may be envisaged.

Hyperbranched polymers are characterized by their degree of polymerization DP=1-b, b being the percentage of non-terminal functions of B which have not reacted with a group A.

Since the condensation is not systematic, unlike for the synthesis of dendrimers (see hereinbelow), the degree of polymerization is less than 100%. A terminal group T on the hyperbranched polymer can be made to react to obtain a particular function at the end of chains.

Several hyperbranched polymers can be combined together, by covalent bonding or another type of bonding, by means of their terminal groups. Such polymers, which are said to be bridged, are included in the definition of the hyperbranched polymers according to the present invention.

Numerous hyperbranched polymers and dendrimers have already been described. Reference may be made, for example, to: D. A. Tomalia et al., Angew. Chem. Int. Engl. 29, 138-175 (1990); N. Ardoin and D. Astruc, Bull. Soc. Chim. Fr. 132, 875-909 (1995); B. I. Voit, Acta Polymer, 46, 87-99 (1995).

Such polymers are described in particular in B. I. Voit, Acta Polymer., 46, 87-99 (1995); EP-682 059; WO—96/14346; WO—96/14345; WO—96/12754. Several hyperbranched polymers can be combined together, by covalent bonding or another type of bonding, by means of their terminal groups.

Such polymers, which are said to be bridged, are included in the definition of the hyperbranched polymers according to the present invention.

“Dendrimers” are macromolecules consisting of monomers which associate by means of an arborescent process around a multifunctional central core.

Dendrimers thus have a fractal (or fractal molecule) structure, consisting of a core, a given number of generations of branches (or wedges), of internal cavities originating from said branches of the molecule, and of terminal functions.

Dendrimers are, structurally, highly branched polymers and oligomers having a well-defined chemical structure.

Dendrimers may be in the form of an assembly of molecules of the same generation, the assembly being referred to as “monodisperse”; they may also be in the form of assemblies of different generations, which are referred to as being “polydisperse”. The definition of dendrimers according to the present invention includes monodisperse dendrimer assemblies as well as polydisperse dendrimer assemblies.

The generations of branches consist of structural units, which are identical for the same generation of branches and which may be identical or different for different generations of branches. All of the junction points of branches of the same generation are located an equal distance from the core; this corresponds to a generation.

The generations of branches extend radially in a geometrical progression from the core. The terminal groups of an n^{th} generation dendrimer are the terminal functional groups of the branches of the n^{th} generation, referred to as the terminal generation.

The definition of dendrimers given above includes molecules bearing symmetrical branching; it also includes molecules bearing non-symmetrical branching, for instance dendrimers in which the branches are lysine groups, in which the branching of one generation of wedges on the preceding generation takes place on the α and ε amines of lysine, which leads to a difference in the length of the wedges of the various branches.

Dendrimers also known as “dense star polymers” or “starburst polymers” or “rod-shaped dendrimers” are included in the present definition of dendrimers. The molecules known as “arborols” and “cascade molecules” are also included in the definition of dendrimers according to the present invention.

Moreover, several dendrimers may be combined together, via a covalent bond or another type of bonding, by means of their terminal groups to give species known as “bridged dendrimers” or “dendrimer aggregates”. Such species are included in the definition of dendrimers according to the present invention.

Dendrimers may be in the form of an assembly of molecules of the same generation, the assembly being referred to as “monodisperse”; they may also be in the form of assemblies of different generations, which are referred to as being “polydisperse”. The definition of dendrimers according to the present invention includes monodisperse dendrimer assemblies as well as polydisperse dendrimer assemblies.

According to another variant of the invention, the thiolated compound used according to the invention denotes a non-polymeric organic compound and may be represented by formula (XVIb):

W(SH)ₙ (XVIb)

in which formula (XVIb):
n denotes an integer greater than or equal to 2, preferably between 2 and 10, preferably between 2 and 5, and
W denotes a linear or branched or (hetero)cyclic, saturated C₂ to C₈₀ polyvalent (at least divalent) radical, an aromatic radical, or a heteroaromatic cyclic radical, W also possibly containing one or more heteroatoms such as O, N or S and/or one or more functions chosen from ester, ketone, amide and urea functions, preferably ester or ketone functions, and/or possibly being substituted with one or more linear or branched C₁-C₁₀ alkyl or linear or branched C₁-C₁₀ alkoxy groups, it being understood that when the radical W is substituted, the thiol functions may be borne by the substituent(s).

The term “cyclic radical” means a hydrocarbon-based or heterocyclic saturated monocyclic radical, a saturated or aromatic polycyclic radical, for example biphenyl, or fused rings, for instance a naphthyl radical.

The molar mass of the compounds of formula (XVIb) is generally between 90 and 1500 g·mol⁻¹.

According to a first embodiment, the compound bearing a thiol unit of formula (XVIb) is such that n=2 and W denotes a linear or branched C₂-C₂₀, preferably C₂-C₁₂ saturated divalent hydrocarbon-based radical.

According to this embodiment, the compound bearing a thiol unit denotes, for example, 1,2-ethanedithiol, 1,2-propanedithiol, 1,3-propanedithiol, 1,4-butanedithiol, 1,6-hexanedithiol, 1,7-heptanedithiol, 1,8-octanedithiol, 1,9-nonanedithiol, 1,10-decanedithiol, 1,12-dodecanedithiol, 2,2-dimethyl-1,3-propanedithiol, 3-methyl-1,5-pentanedithiol, or 2-methyl-1,8-octanedithiol.

According to another embodiment, the compound bearing a thiol unit of formula (XVIb) is such that n=3 and W denotes a linear or branched C₃-C₂₀, preferably linear or branched C₂-C₁₂ saturated trivalent hydrocarbon-based radical.

According to this other embodiment, the compound bearing a thiol unit may be chosen, for example, from 1,1,1-tris(mercaptomethyl)ethane, 2-ethyl-2-mercaptomethyl-1,3-propanedithiol, and 1,2,3-propanetrithiol.

According to one embodiment, the compound bearing a thiol unit of formula (XVIb) is such that n=2 or 3 and W denotes a linear or branched C₃-C₂₀, preferably linear or branched C₂-C₁₂, saturated divalent or trivalent hydrocarbon-based radical, said radical containing one or more non-adjacent heteroatoms chosen from O and S.

According to this third embodiment, the compound bearing a thiol unit may be chosen, for example, from: C₂-C₁₂ bis(mercaptoalkyl) ethers and sulfides such as bis(2-mercaptoethyl) ether, bis(2-mercaptoethyl) sulfide, bis(2-mercaptoethylthio-3-mercaptopropane) sulfide, (C₁-C₅)bis(2-mercapto(C₁-C₃)alkylthio)alkanes or (C₁-C₅)bis(2-mercapto(C₁-C₃)alkylthio)mercaptoalkanes, for instance bis(2-mercaptoethylthio)methane, 1,2-bis(2-mercaptoethylthio)ethane, 1,3-bis(2-mercaptoethylthio)propane, 1,2-bis(2-mercaptoethylthio)propanethiol, 1,2-bis(2-mercaptoethyl)thio-3-mercaptopropane and 1,2,3-tris(2-mercaptoethylthio)propane.

Preferably, according to this embodiment, compound (XVIb) is chosen from 1,2-bis(2-mercaptoethylthio)propanethiol, 1,2,3-tris(2-mercaptoethylthio)propane and tetrakis(2-mercaptoethylthiomethyl)methane.

According to one embodiment, the compound bearing a thiol unit of formula (XVIb) is such that n denotes an integer greater than or equal to 2 and W denotes a linear or branched C₃-C₂₀, preferably linear or branched C₂-C₁₂, hydrocarbon-based saturated multivalent (at least divalent) radical, said radical containing at least one ester function.

According to this embodiment, the compound bearing a thiol unit may be chosen from: esters of polyols (glycols, triols, tetraols, pentaols, hexaols) and of C₁-C₆ mercaptocarboxylic acid, such as ethylene glycol bis(2-mercaptoacetate), ethylene glycol bis(3-mercaptopropionate), ethylene glycol bis(thioglycolate), trimethylolpropane tris(thioglycolate), trimethylolpropane tris(β-mercaptopropionate), pentaerythrityl tetrakis(thioglycolate), pentaerythrityl tetrakis(β-mercaptopropionate), dipentaerylthrityl hexakis(β-mercaptoproprionate), trimethylolpropane tris(2-mercaptoacetate), trimethylolpropane tris(3-mercaptopropionate), pentaerythrityl tetrakis(2-mercaptoacetate), pentaerythrityl tetrakis(3-mercaptopropionate), pentaerythrityl tetrakis(3-mercaptobutanate), and dipentaerythrityl hex-3-mercaptopropionate.

Preferably, according to this embodiment, the compound bearing a thiol unit is chosen from trimethylolpropane tris(2-mercaptoacetate), trimethylolpropane tris(3-mercaptopropionate), pentaerythritol tetrakis(2-mercaptoacetate), pentaerythritol tetrakis(3-mercaptopropionate), pentaerythritol tetrakis(3-mercaptobutanate) and dipentaerythritol hex-3-mercaptopropionate.

Particularly preferably, the compound bearing a thiol unit is pentaerythritol tetrakis(3-mercaptopropionate).

According to one embodiment, the compound bearing a thiol unit, of formula (XVIb), is such that n=4 and W denotes a branched C₄-C₂₀, preferably C₈-C₁₄, hydrocarbon-based saturated tetravalent radical interrupted with one or more non-adjacent sulfur atoms.

According to this embodiment, the compound bearing a thiol unit may be chosen from tetrakis(2-mercaptoethylthiomethyl)methane and bis(2-mercaptoethylthio-3-mercaptopropane) sulfide.

According to one embodiment, the compound bearing a thiol unit, of formula (XVIb), is such that n=2 and W denotes a hydrocarbon-based cyclic divalent radical optionally containing one or more non-adjacent sulfur atoms, optionally substituted with one or more linear or branched C₁-C₁₀ alkyl radicals.

According to this embodiment, the compound bearing a thiol unit may be chosen, for example, from 1,4-cyclohexanedithiol, 1,4-bis(mercaptomethyl)cyclohexane, 1,1-cyclohexanedithiol, 1,2-cyclohexanedithiol, 1,1-bis(mercaptomethyl)cyclohexane, and 2,5-dimercapto-1,4-dithiane.

According to one embodiment, the compound bearing a thiol unit, of formula (XVIb), is such that n=3 and W denotes a substituted isocyanurate-type cyclic radical.

According to this embodiment, the compound bearing a thiol unit may be chosen from polythiols of the isocyanurate class, described in U.S. Pat. Nos. 3,676,440 and 2,011,023 0585, such as tris((mercaptopropionyloxy)ethyl) isocyanurate.

According to this embodiment, the compound bearing a thiol unit is preferably tris((mercaptopropionyloxy)ethyl) isocyanurate.

According to one embodiment, the compound bearing a thiol unit, of formula (XVIb), is such that n=2 or 3 or 4 and W denotes an aromatic radical optionally substituted with one or more identical or different radicals of C₁-C₁₀ alkyl or C₁-C₁₀ alkoxy type, it being understood that, when the radical W is substituted, the thiol functions may be borne by the substituent(s).

According to this embodiment, the compound bearing a thiol unit may be chosen, for example, from the following compounds: 1,2-dimercaptobenzene, 1,3-dimercaptobenzene, 1,4-dimercaptobenzene, 1,2-bis(mercaptomethyl)benzene, 1,3-bis(mercaptomethyl)benzene, 1,4-bis(mercaptomethyl)benzene, 1,2-bis(2-mercaptoethyl)benzene, 1,3-bis(2-mercaptoethyl)benzene, 1,4-bis(2-mercaptoethyl)benzene, 1,2-bis(2-mercaptoethyleneoxy)benzene, 1,3-bis(2-mercaptoethyleneoxy)benzene, 1,4-bis(2-mercaptoethyleneoxy)benzene, 1,2,3-trimercaptobenzene, 1,2,4-trimercaptobenzene, 1,3,5-trimercaptobenzene, 1,2,3-tris(mercaptomethyl)benzene, 1,2,4-tris(mercaptomethyl)benzene, 1,3,5-tris(mercaptomethyl)benzene, 1,2,3-tris(2-mercaptoethyl)benzene, 1,2,4-tris(2-mercaptoethyl)benzene, 1,3,5-tris(2-mercaptoethyl)benzene, 1,2,3-tris(2-mercaptoethyleneoxy)benzene, 1,2,4-tris(2-mercaptoethyleneoxy)benzene, 1,3,5-tris(2-mercaptoethyleneoxy)benzene, 1,2,3,4-tetramercaptobenzene, 1,2,3,5-tetramercaptobenzene, 1,2,4,5-tetramercaptobenzene, 1,2,3,4-tetrakis(mercaptomethyl)benzene, 1,2,3,5-tetrakis(mercaptomethyl)benzene, 1,2,4,5-tetrakis(mercaptomethyl)benzene, 1,2,3,4-tetrakis(2-mercaptoethyl)benzene, 1,2,3,5-tetrakis(2-mercaptoethyl)benzene, 1,2,4,5-tetrakis(2-mercaptoethyl)benzene, 1,2,3,4-tetrakis(2-mercaptoethyleneoxy)benzene, 1,2,3,5-tetrakis(2-mercaptoethyleneoxy)benzene, 1,2,4,5-tetrakis(2-mercaptoethyleneoxy)benzene, 2,2′-dimercaptobiphenyl, 4,4′-dimercaptobiphenyl, 4,4′-dimercaptobibenzyl, 2,5-toluenedithiol, 3,4-toluenedithiol, 1,4-naphthalenedithiol, 1,5-naphthalenedithiol, 2,6-naphthalenedithiol, 2,7-naphthalenedithiol, 2,4-dimethylbenzene-1,3-dithiol, 4,5-dimethylbenzene-1,3-dithiol, 9,10-anthracenedimethanethiol, 1,3-bis(2-mercaptoethylthio)benzene, 1,4-bis(2-mercaptoethylthio)benzene, 1,2-bis(2-mercaptoethylthiomethyl)benzene, 1,3-bis(2-mercaptoethylthiomethyl)benzene, 1,4-bis(2-mercaptoethylthiomethyl)benzene, 1,2,3-tris(2-mercaptoethylthio)benzene, 1,2,4-tris(2-mercaptoethylthio)benzene, 1,3,5-tris(2-mercaptoethylthio)benzene, 1,2,3,4-tetrakis(2-mercaptoethylthio)benzene, 1,2,3,5-tetrakis(2-mercaptoethylthio)benzene, 1,2,4,5-tetrakis(2-mercaptoethylthio)benzene and 3,4-thiophenedithiol.

According to this embodiment, compound (XVI) is chosen from 1,2,3-trimercaptobenzene, 1,2,4-trimercaptobenzene, 1,3,5-trimercaptobenzene, 1,2,3-tris(mercaptomethyl)benzene, 1,2,4-tris(mercaptomethyl)benzene, 1,3,5-tris(mercaptomethyl)benzene, 1,2,3-tris(2-mercaptoethyl)benzene, 1,2,4-tris(2-mercaptoethyl)benzene, 1,3,5-tris(2-mercaptoethyl)benzene, 1,2,3-tris(2-mercaptoethyleneoxy)benzene, 1,2,4-tris(2-mercaptoethyleneoxy)benzene, 1,3,5-tris(2-mercaptoethyleneoxy)benzene, 1,2,3,4-tetramercaptobenzene, 1,2,3,5-tetramercaptobenzene, 1,2,4,5-tetramercaptobenzene, 1,2,3,4-tetrakis(mercaptomethyl)benzene, 1,2,3,5-tetrakis(mercaptomethyl)benzene, 1,2,4,5-tetrakis(mercaptomethyl)benzene, 1,2,3,4-tetrakis(2-mercaptoethyl)benzene, 1,2,3,5-tetrakis(2-mercaptoethyl)benzene, 1,2,4,5-tetrakis(2-mercaptoethyl)benzene, 1,2,3,4-tetrakis(2-mercaptoethyleneoxy)benzene, 1,2,3,5-tetrakis(2-mercaptoethyleneoxy)benzene, 1,2,4,5-tetrakis(2-mercaptoethyleneoxy)benzene, 1,2,3-tris(2-mercaptoethylthio)benzene, 1,2,4-tris(2-mercaptoethylthio)benzene, 1,3,5-tris(2-mercaptoethylthio)benzene, 1,2,3,4-tetrakis(2-mercaptoethylthio)benzene, 1,2,3,5-tetrakis(2-mercaptoethylthio benzene, 1,2,4,5-tetrakis(2-mercaptoethylthio)benzene and 3,4-thiophenedithiol.

According to a ninth embodiment, the compound bearing a thiol unit, of formula (XVIb), is such that n=2 or 3 or 4 and W denotes a triglyceride of a fatty acid or a plant oil, which are optionally substituted, it being understood that when the radical W is substituted, the thiol functions may be borne by the substituent(s).

According to another particular embodiment of the invention, the thiolated and/or hydroxylated compound is chosen from thiolated hydroxylated and/or thiolated fatty acid triglyceride derivatives, such as those of formula (XVIIb):

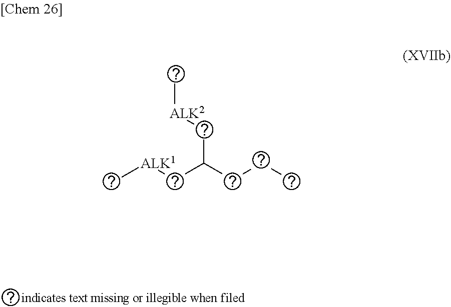

in which formula (XVIIb):
R¹, R² and R³, which may be identical or different, represent a hydrogen atom or a hydroxyl or thiol group, preferably a thiol group;
ALK¹, ALK² and ALK³, which may be identical or different, represent a (C₁-C₃₀)alkylene group optionally substituted with one or more hydroxyl or thiol groups, preferably thiol groups;
X¹, X² and X³, which may be identical or different, preferably identical, represent a group —C(Y)—Y′— or —Y′—C(Y)— with Y and Y′, which may be identical or different, preferably identical, representing a heteroatom such as O, S and N, preferably O.

Preferably, the compounds of formula (XVIIb) are such that:
R¹, R² and R³ represent a hydrogen atom;
ALK¹ represents a (C₁₀-C₂₄)alkylene and particularly (C₁₄-C₂₀)alkylene group, which is preferably linear;
ALK² represents a (C₁₀-C₂₄)alkylene and particularly (C₁₄-C₂₀)alkylene group, which is preferably linear, substituted with one or more thiol groups;
ALK³ represents a (C₁₀-C₂₄)alkylene and particularly (C₁₄-C₂₀)alkylene group, which is preferably linear, substituted with one or more thiol groups, preferably two thiol groups; and/or
X¹, X² and X³, which are identical, represent a —C(O)—O— or —O—C(O)— group.

According to this embodiment, the compound bearing a thiol unit may be chosen, for example, from: fatty acid triglycerides or plant oils modified with thiol groups by chemical reaction, for instance thiolated soybean oils and hydroxylated and thiolated soybean oils, notably the Polymercaptan® products from the company Chevron Phillips, such as Polymercaptan 407 (mercapto hydroxy soybean oil) and Polymercaptan 358 (mercaptanized soybean oil) of formula (XVIIIb) below:

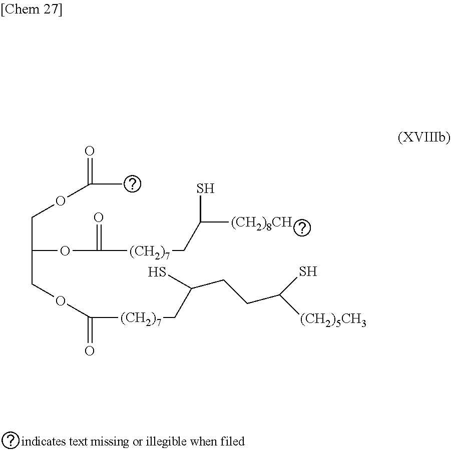

More preferentially, the thiolated hydroxylated and/or thiolated fatty acid triglyceride derivatives of formula (XVIIb) are such as those of formula (XVIIIb) above.

According to a particular embodiment of the invention, the thiolated and/or hydroxylated compound is chosen from polyhydroxylated, polythiolated, or (poly)hydroxylated and (poly)thiolated compounds containing several hydroxyl and/or thiol groups, and having a weight-average molecular weight ranging from 500 to 1 000 000 g·mol⁻¹, preferably ranging from 500 to 500 000 g·mol⁻¹, and preferentially ranging from 500 to 100 000 g·mol⁻¹.

According to this variant, preference will be given to the compounds of formula (XVIb) for which n denotes an integer greater than or equal to 3, between 3 and 10 and more preferentially between 3 and 5.

Preferably, according to this variant, the compounds of formula (XVIb) are chosen from compounds of the second embodiment, or from compounds of the third embodiment; or from compounds of the fourth embodiment, in particular such as trimethylolpropane tris(2-mercaptoacetate), trimethylolpropane tris(3-mercaptopropionate), pentaerythrityl tetrakis(2-mercaptoacetate), pentaerythrityl tetrakis(3-mercaptopropionate), pentaerythrityl tetrakis(3-mercaptobutanate) or dipentaerythrityl hex-3-mercaptopropionate, or from compounds of the fifth embodiment, or from compounds of the seventh embodiment, in particular such as tris((mercaptopropionyloxy)ethyl) isocyanurate.

Particularly preferably, according to this variant, the compounds of formula (XVI) are chosen from trimethylolpropane tris(2-mercaptoacetate), trimethylolpropane tris(3-mercaptopropionate), pentaerythrityl tetrakis(2-mercaptoacetate), pentaerythrityl tetrakis(3-mercaptopropionate), pentaerythrityl tetrakis(3-mercaptobutanate), dipentaerythrityl hex-3-mercaptopropionate or tris((mercaptopropionyloxy)ethyl) isocyanurate.

According to another variant, the compound bearing a thiol unit according to the invention denotes a polymeric compound and may be represented by formula (XIXb):

POL(SH)ₙ (XIXb)

in which formula (XIXb):
n denotes an integer greater than or equal to 5, preferably between 5 and 5000, preferably from 5 to 1000, and
POL denotes a carbon-based or silicone-based multivalent (at least pentavalent) polymeric radical, POL also possibly containing one or more heteroatoms such as O, N or S, and/or one or more functions chosen from ester, ketone, amide, urea and carbamate functions, and/or possibly being substituted with one or more linear or branched C₁-C₁₀ alkyl or linear or branched C₁-C₁₀ alkoxy groups, it being understood that when POL is substituted, the thiol functions may be borne by the substituent(s).

The molar mass of the compounds of formula (XIXb) is generally between 500 and 400 000 g·mol⁻¹ and preferably between 500 and 150 000 g·mol⁻¹.

In a particular embodiment, POL denotes a multivalent homopolymer or copolymer radical.

In a particular embodiment, POL denotes a polymeric radical of star, comb, brush or dendritic type.

The POL radical may be of natural origin (such as polysaccharides, peptides) or synthetic origin (such as acrylic polymers, polyesters, polyglycols).

The thiol functions (—SH) may be terminal and/or side groups.

According to a first embodiment, the thiolated compound of formula (XIXb) is such that POL denotes a hydrocarbon-based polymeric radical.

Examples that may be mentioned include the polymers described in the following articles: Polymers containing groups of biological activity, C. G. Overberger et al., Polytechnic Institute of Brooklyn, http://pac.iupac.org/publications/pac/pdf/1962/pdf/0402x0521.pdf and Mercaptan-containing polymers, Advances in Polymer Science, volume 15, 1974, pages 61-90.

In particular, mention may be made of the compounds bearing a thiol unit, of formula (XIXb), such as poly(vinylmercaptan), poly(4-mercaptostyrene), poly(vinylbenzylmercaptan), poly(4-mercaptostyrene)-co-poly(methyl methacrylate), and also polymers containing amide functions, such as poly(thiolated hexamethylene adipamide).

The compounds of formula (XIXb) also denote proteins and peptides with thiol units, for instance the structures represented in the following table:

TABLE 3

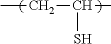

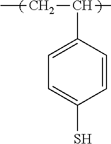

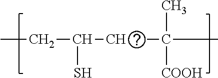

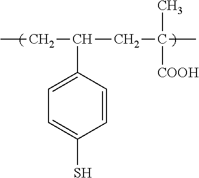

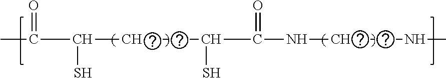

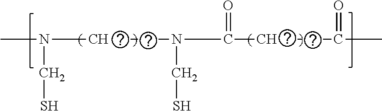

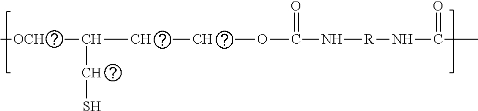

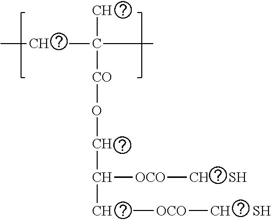

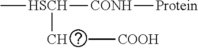

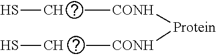

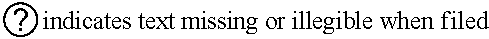

The thiolated compounds of formula (XIXb) also denote compounds such that POL denotes a radical termed dendrimer or polymer which is branched or hyperbranched, and the thiol groups are end groups. As examples, mention may be made of the polymers described in the article Progress in Organic Coatings, volume 63, issue 1, July 2008, pages 100-109.

As an example of a synthesis of such polymers, mention may be made of the synthesis described in said article in which the polymer Boltorn H40 is transformed into a thiolated polymer of formula (XIXb) according to the scheme below:

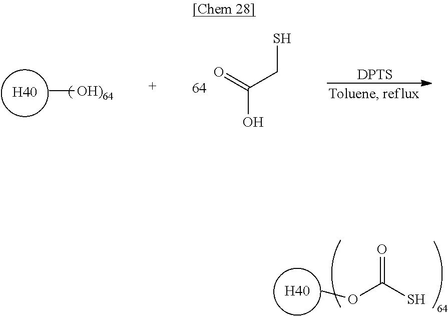

The structure of the thiolated polymer (XIXb) obtained is given below:

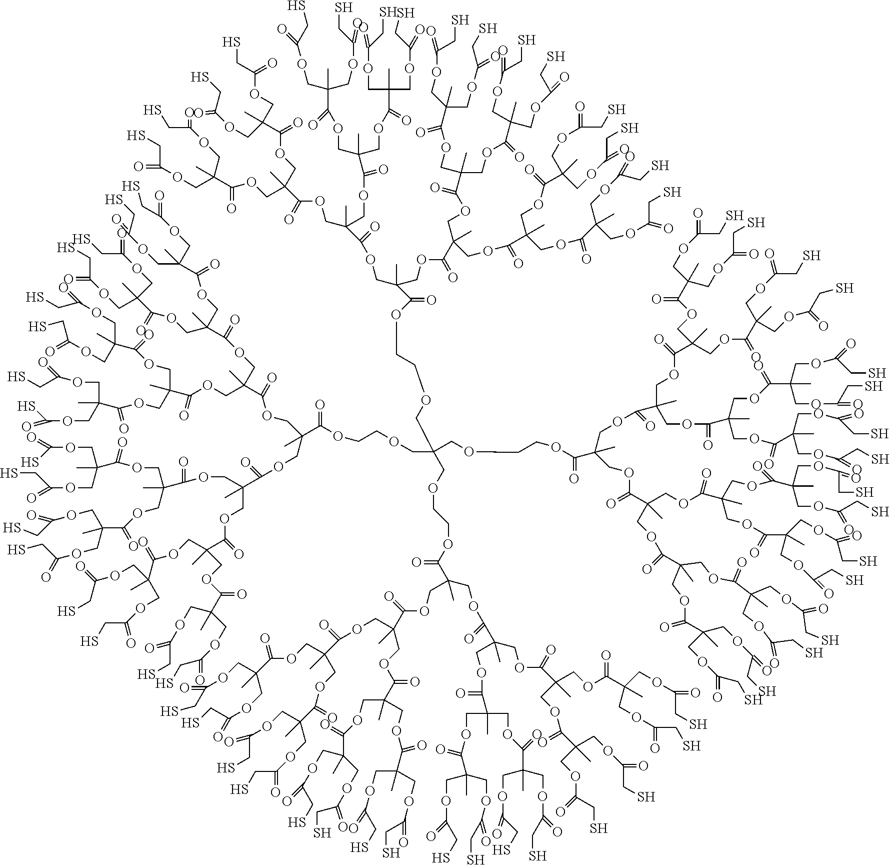

The compound bearing a thiol unit, of formula (XIX), may also denote a hyperbranched or dendritic polymer modified with thiol functions, as described in patent application FR 2 761 691.

As examples of hyperbranched polymers and dendrimers, mention may be made of the compounds including thiol functional groups of formula (XXb) below:

HS-A-C(Y)—X— (XXb)

in which formula (XXb):
Y represents an oxygen or sulfur atom or a group NR′;
X represents i) an oxygen atom or ii) a group —N(R′)— in which R′ is chosen from a) a hydrogen atom, b) a linear or branched, saturated or unsaturated C₁-C₆ alkyl group, c) a linear or branched, saturated or unsaturated C₁-C₆ monohydroxyalkyl or polyhydroxyalkyl group, d) a C₁-C₆ aminoalkyl group or a polyalkyleneimine group; preferably, X represents —N(R′)— with R′ representing a hydrogen atom or a (C₁-C₄)alkyl group such as methyl; and
A represents a linear, branched or cyclic, saturated or unsaturated (C₁-C₁₂)alkylene group; this group being optionally interrupted with one or more heteroatoms such as 0, S or N and/or optionally substituted with one or more groups chosen from amino (—NH₂), acylamino (—N(H)—C(O)—R) or aminoacyl (RN(H)—C(O)—) in which R represents a linear, branched or cyclic, saturated or unsaturated C₁-C₁₀ alkyl, carboxyl (—C(O)OH) or ester (—C(O)—OR) group in which R represents a linear, branched or cyclic, saturated or unsaturated C₁-C₁₀ alkyl group.

Preferably, the thiolated polymers according to the invention are chosen from hyperbranched polymers, and notably polyethyleneimine including at least one group chosen from the groups of formula (XXb) as defined previously.

Preferably, Y represents an oxygen atom. Preferably, the heteroatoms are chosen from oxygen and nitrogen (O and N).

Preferably, A is a methylene, ethylene, propylene, methylpropylene, ethylpropylene, tetramethylene, pentamethylene, hexamethylene or phenylene group.

Advantageously, A represents a radical corresponding to one of the formulae (a) to (d) below:

### [Chem 30]

—CHR¹—CHR²—CHR³— (a)

—CHR′¹—CHR′—CHR′³—CHR′⁴— (b)

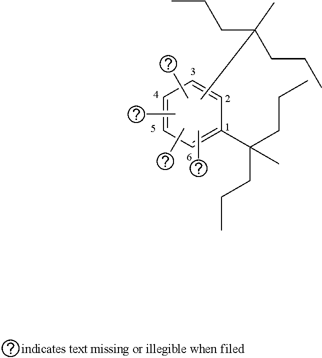

(c)

—(CHR′″¹)ₖ—(CHR′″²—CH(CO₂H)—NH— (d)

in which formulae (a), (b), (c) and (d):
R¹, R², R³, R′¹, R′², R′³ and R′⁴, R′″¹ and R′², which may be identical or different, represent: a hydrogen atom; a linear, branched or cyclic, saturated or unsaturated C₁-C₆ alkyl radical; an amino radical (—NH₂); a carboxylic acid radical (—C(O)OH); a C₁-C₁₀ alkylamino radical; a C₁-C₁₀ acylamino radical;
R″¹, R″², R″³ and R″⁴, which may be identical or different, represent a hydrogen atom or a linear or branched, saturated or unsaturated C₁-C₄ alkyl group; the arrows indicating the positions of the substitutions; and
k is an integer, preferentially 0 or 1;

### [Chem 31]

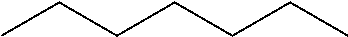

represents the point of attachment to the rest of the molecule on the phenylene group in position 1-2, or 1-3, or 1-4; it being understood that the radicals R″₁, R″₂, R″₃ and R″₄ are then positioned on the carbon atoms 3, 4, 5, 6, or 2, 4, 5 or 6 or 2, 3, 5, 6, respectively.

According to a preferred embodiment of the invention, the thiolated polymers are hyperbranched polymers and dendrimers including functional groups of formula (XXb) such that A is chosen from:
—CH₂—CH(CO₂H)—NH— and Y represents an oxygen atom;
—(CH₂)₂—(CH₃CONH)CH— and Y represents an oxygen atom;
—(CH₂)₃— and Y represents an oxygen atom or an NH group.

In particular, A is the propylene group —CH₂—CH₂—CH₂— and Y represents an oxygen atom, the group of formula (XXb) then corresponding to formula (XXIb) below:

HS—CH₂—CH₂—CH₂—C(O)—X— (XXIb)

in which formula (XXIb) X is as defined in formula (XXb); preferably, X represents —N(R′)— with R′ representing a hydrogen atom or a (C₁-C₄)alkyl group such as methyl.

Preferentially, in formulae (XXb) and (XXIb), X is chosen from an oxygen atom and an NH group.

According to one of the preferred embodiments of the invention, the thiolated polymers are as described in FR 2 853 533, that is to say poly-N-α- and N—ε-lysine and ornithine of formula I, bearing a thiol function, which may be obtained from poly-N-α- and N—ε-lysine and ornithine by reaction with a thiolactone, for instance thiobutyrolactone (dihydrothiophen-2(3H)-one).

According to a preferred embodiment of the invention, the hyperbranched polymers and dendrimers that are useful in the invention include functional groups corresponding to formula (XXIIb):

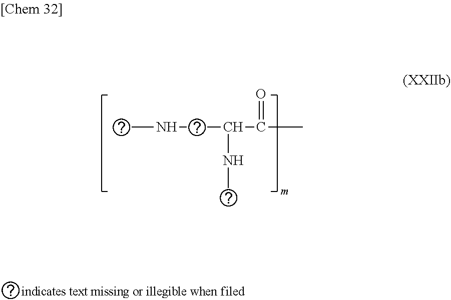

in which formula (XXIIb): p is different from p′ and p, p′ are equal to 0 or 1;
n is equal to 3 or 4;
if p′ is equal to 0, then the neighbouring NH is engaged in an N—ε polymerization;
if p is equal to 0, then the neighbouring NH is engaged in an N-α polymerization;
if p or p′ is equal to 1, then R or R′ represents —B—SH, with B representing a saturated or unsaturated, linear or branched C₁-C₃₀ hydrocarbon-based chain which may be interrupted with one or more heteroatoms or groups, alone or in combination, such as: —N(R¹)—, —O—, —S(O)ᵣ, —C(O)—, —C(S)— or —C(NR¹)—, with r being equal to 0, 1 or 2, and/or with one or more 5-, 6- or 7-membered aryl, heteroaryl, cycloalkyl or heterocycloalkyl which may be substituted with one or more halogen atoms or groups from among: hydroxyl, amino, carboxyl, (di)(C₁-C₈)alkylamino, (C₁-C₈)acylamino, (C₁-C₈)acyloxy, (C₁-C₈)alkyloxycarbonylamino, (C₁-C₈)alkylaminocarbonyloxy and (C₁-C₈)alkylaminocarbonyl; given that R or R′ may, in part only, also represent a hydrogen atom, and/or —C(NH)— and salts thereof, and/or —C(NH)—N(H)—C(NH)—NH₂ and salts thereof,
R₁ represents a hydrogen atom or a (C₁-C₈)alkyl, (C₁-C₈)acyl, (C₁-C₈)alkyloxycarbonyl, (C₁-C₈)alkylaminocarbonyl or halo group;
B may also represent an optionally substituted 5-, 6- or 7-membered aryl, heteroaryl, cycloalkyl or heterocycloalkyl group;
m represents an integer ranging from 3 to 10 000.

Preferably, the degree of thiol function grafting is greater than or equal to 1%.

Advantageously, the poly N-α- and N—ε-lysine and ornithine corresponding to formula (XXIIb) have: 5<m<1000.

The term “theoretical degree of thiol function grafting” represents the theoretical percentage of lysine or ornithine units bearing the thiol function in the compound of formula (XXIIb). Examples of hyperbranched polymers that may be mentioned most particularly include hyperbranched thiolated polyethyleneimines, such as those described in patent application EP 103 759 with a molecular molar mass ranging from 30×10⁴ to 50×10⁴ g·mol⁻¹. These polymers are prepared according to methods that are conventional to those skilled in the art, such as the methods described in French patent application FR 2 761 691 and EP 1 037 938. According to a particular embodiment of the invention, the dendrimer(s) and branched or hyperbranched polymer(s) bear thiol terminal groups, such as the Boltorn™ dendritic polythiols from the company BASF esterified with compounds such as thioglycolic acid and described in the literature. Polymers such as polypropylene ether glycol bis(β-mercaptopropionate) may also be used according to the invention. They are prepared via the methods known to those skilled in the art. Mention may be made, for example, of the preparation method by esterification reaction of polypropylene ether glycol such as Pluracol P201 sold by the company Wyandotte Chemical Corp. and β-mercaptopropionic acid.

According to a particular embodiment of the invention, the hydroxylated and/or thiolated polymers are polyethoxylated of formula (XXIIIb), and also the optical isomers thereof, the acid or base salts thereof, and the solvates thereof such as hydrates:

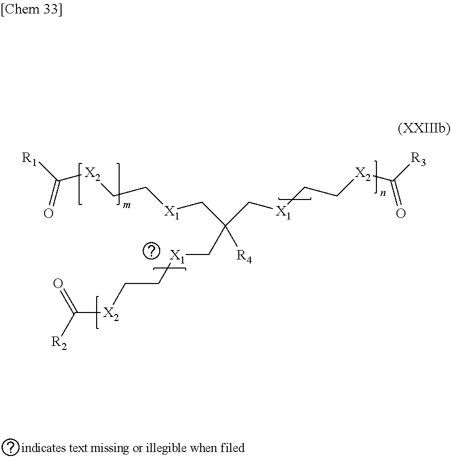

in which formula (XXIIIb):
R₁, R₂ and R₃, which may be identical or different, preferably identical, represent a hydroxy(C₁-C₆)alkyl or thio(C₁-C₆)alkyl group, preferably a thio(C₁-C₆)alkyl group;
R₄ represents a hydrogen atom or a group from among: hydroxyl, thiol, amino or (C₁-C₆)alkyl, preferably (C₁-C₄)alkyl such as ethyl;
X₁ and X₂, which may be identical or different, preferably identical, represent an oxygen or sulfur atom, or amino, preferably oxygen;
m, n and l, which may be identical or different, represent an integer greater than or equal to 1.

The thiolated polymer compounds of formula (XXIIIb) are commercially available. Mention may be made, for example, of the products Thiocure® from the company Bruno Brock, Thiocure® ETTMP 1300 (Ethoxylated-Trimethylolpropane Tri-3-Mercaptopropionate (CAS #345352-19-4) and Thiocure® ETTMP 700 (Ethoxylated-Trimethylolpropane Tri-3-Mercaptopropionate (CAS #345352-19-4).

According to a preferred embodiment, the crosslinking agent R is a (poly)thiolated and/or (poly)hydroxylated compound, in particular chosen from non-polymeric (poly)thiolated and/or (poly)hydroxylated compounds such as polyhydroxylated compounds (liposoluble polyol), polythiolated compounds (dithiol compounds), hydroxylated and/or thiolated alkoxysiloxanes, silicas functionalized with radicals, notably of the alkyl type, substituted with thiol functions, latices bearing thiol groups, particles coated with compound(s) bearing at least two thiol functions and polymeric (poly)thiolated and/or (poly)hydroxylated compounds such as homopolymers, copolymers, star, comb, brush and dendritic compounds with hydroxyl and/or thiol units, which are preferably organic or silicon-based.

Preferably, the polymeric (poly)thiolated and/or (poly)hydroxylated compounds are chosen from (di)ol polymers, in particular polyolefin (poly)ols, polydi(C₁-C₆)alkylsiloxane (poly)ols, polyester (poly)ols, hydroxylated thiolated and/or thiolated fatty acid triglyceride derivatives, amine thiols derived from dendrimers or polyethyleneimines (PEI) and silicone thiols.

Preferably, the polymeric (poly)thiolated and/or (poly)hydroxylated compounds are chosen from polydimethylsiloxane diols such as hydroxy-terminated polydimethylsiloxanes; thiolated poly(C₁-C₄)alkylsiloxanes such as polydimethylsiloxanes having at least two thiol groups; and fatty acid triglycerides or plant oils modified with thiol groups by chemical reaction.

Preferably, the (poly)thiolated and/or (poly)hydroxylated compounds used according to the invention are chosen from polydimethylsiloxanes including at least two thiol groups.

### (Poly)Carbonyl Crosslinking Agents

According to a particular embodiment, the crosslinking agent R is a (poly)carbonyl compound.

In particular, the (poly)carbonyl compound is chosen from terephthalaldehyde, 5,5-dimethyl-1,3-cyclohexanedione, phenylglyoxal, isophthalaldehyde, 4-acetylbenzaldehyde, 4,4-diformyltriphenylamine, 2-acetylbenzaldehyde, 3-(2-furoyl)quinoline-2-carboxaldehyde 3-(2-furoyl)quinoline-2-carboxaldehyde, 3-acetylbenzaldehyde, 9-(2-ethylhexyl)carbazole-3,6-dicarboxaldehyde, phthaldialdehyde, 1,3-cyclohexanedione, 4,4′-biphenyldicarboxaldehyde, benzene-1,3,5-tricarboxaldehyde, and oxidized inulin.

In particular, the (poly)carbonyl compound is chosen from terephthalaldehyde, 5,5-dimethyl-1,3-cyclohexanedione, phenylglyoxal, isophthalaldehyde, 4-acetylbenzaldehyde, 4,4-diformyltriphenylamine, 2-acetylbenzaldehyde, 3-(2-furoyl)quinoline-2-carboxaldehyde, 3-(2-furoyl)quinoline-2-carboxaldehyde, 3-acetylbenzaldehyde, 9-(2-ethylhexyl)carbazole-3,6-dicarboxaldehyde, phthaldialdehyde, 1,3-cyclohexanedione, 4,4′-biphenyldicarboxaldehyde, benzene-1,3,5-tricarboxaldehyde, oxidized inulin, and terephthalaldehyde, preferably terephthalaledhyde.

According to this embodiment, the (poly)carbonyl compound is associated in its implementation with an amine catalyst as described, for example, in the articles Progress in coating 129, 21-25 (2019) and Progress in coating 135, 510-516 (2019); preferably, the amine catalyst(s) are chosen from piperidine, DMAP (dimethylaminopyridine), DBU (1,8-diazabicyclo[5.4.0]undec-7-ene), DABCO (1,4-diazabicyclo[2.2.2]octane) and DBN (1,5-diazabicyclo[4.3.0]non-5-ene), more preferentially chosen from DBU (1,8-diazabicyclo[5.4.0]undec-7-ene), DABCO (1,4-diazabicyclo[2.2.2]octane) and DBN (1,5-diazabicyclo[4.3.0]non-5-ene), and in particular the catalyst is DBU (1,8-diazabicyclo[5.4.0]undec-7-ene).

### (Poly)Acrylate Crosslinking Agents

According to a particular embodiment, the crosslinking agent is a (poly)acrylate compound.

More preferably the crosslinker is selected from the (poly)acrylate, especially for keratin fibers, and particularly be chosen from 1,3-butanediol diacrylate, 1,4-butanediol diacrylate, bis(trimethylolpropane) tetraacrylate, glyceryl 1,3-diglycerolate diacrylate, glyceryl propoxylate (1PO/OH) triacrylate, 1,6-hexanediol diacrylate, 1,6-hexanediol ethoxylate diacrylate, hydroxypivalyl hydroxypivalate, neopentyl glycol diacrylate, neopentyl glycol propoxylate (1PO/OH) diacrylate, pentaerythrityl tetraacrylate, pentaerythrityl triacrylate, poly(propylene glycol) diacrylate, tricyclo[5.2.1.02,6]decanedimethanol diacrylate, trimethylolpropane ethoxylate (1EO/OH) methyl ether diacrylate, trimethylolpropane propoxylate triacrylate, trimethylolpropane triacrylate, trimethylolpropane trimethacrylate, tri(propylene glycol) diacrylate, trimethylolpropane triacrylate, and tris[2-(acryloyloxy)ethyl]isocyanurate such as trimethylpropane triacrylate.

More particularly, the (poly)acrylate compound may be chosen from 1,3-butanediol diacrylate, 1,4-butanediol diacrylate, bis(trimethylolpropane) tetraacrylate, glyceryl 1,3-diglycerolate diacrylate, glyceryl propoxylate (1PO/OH) triacrylate, 1,6-hexanediol diacrylate, 1,6-hexanediol ethoxylate diacrylate, hydroxypivalyl hydroxypivalate, neopentyl glycol diacrylate, neopentyl glycol propoxylate (1PO/OH) diacrylate, pentaerythrityl tetraacrylate, pentaerythrityl triacrylate, poly(propylene glycol) diacrylate, tricyclo[5.2.1.02,6]decanedimethanol diacrylate, trimethylolpropane ethoxylate (1EO/OH) methyl ether diacrylate, trimethylolpropane propoxylate triacrylate, trimethylolpropane triacrylate, trimethylolpropane trimethacrylate, tri(propylene glycol) diacrylate, trimethylolpropane triacrylate, and tris[2-(acryloyloxy)ethyl]isocyanurate.

More particularly the (poly)acrylate compound is trimethylolpropane triacrylate.

The (poly)acrylate compound may also be chosen from N,N′-methylenebisacrylamide.

According to this embodiment, the (poly)acrylate compound is associated in its implementation with an amine catalyst as described, for example, in the articles Progress in coating 129, 21-25 (2019) and Progress in coating 135, 510-516 (2019); preferably, the amine catalyst(s) are chosen from piperidine, DMAP (dimethylaminopyridine), DBU (1,8-diazabicyclo[5.4.0]undec-7-ene), DABCO (1,4-diazabicyclo[2.2.2]octane) and DBN (1,5-diazabicyclo[4.3.0]non-5-ene), more preferentially chosen from DBU (1,8-diazabicyclo[5.4.0]undec-7-ene), DABCO (1,4-diazabicyclo[2.2.2]octane) and DBN (1,5-diazabicyclo[4.3.0]non-5-ene), and in particular the catalyst is DBU (1,8-diazabicyclo[5.4.0]undec-7-ene).

### Metal Salt Crosslinking Agents:

According to another particular embodiment, the crosslinking agent R is a metal salt chosen from alkali metal salts, alkaline-earth metal salts such as magnesium salts, transition metal salts, post-transition metal salts such as aluminium or tin salts, metalloid salts such as boron salts, hydrates thereof and mixtures thereof.

Preferably, the metal salt(s) are chosen from post-transition metal salts such as aluminium salts, hydrates thereof and mixtures thereof.

The term “metal salt” means a salt resulting notably from the action of an acid on a metal, in particular a transition metal, post-transition metal, metalloid or alkali or alkaline-earth metal.

The metal salt(s) may be in the form of hydrates.

The metal salt(s) may be organic or inorganic.

The term “organic metal salt” means a salt resulting notably from the action of an organic acid on a metal, in particular transition metals, post-transition metals, metalloids, or alkaline or alkaline-earth metals, preferably resulting from the action of a carboxylic acid on a metal.

Preferably the metal salt(s) are chosen from organic metal salts, hydrates thereof and mixtures thereof.

The term “inorganic metal salt” means a salt resulting notably from the action of an inorganic acid on a metal, in particular a transition metal, a post-transition metal, a metalloid or an alkali or alkaline-earth metal.

The term “inorganic acid” means an acid which does not include any carbon atoms, apart from carbonic acid.

According to a particular embodiment of the invention, the inorganic metal salt(s) may be chosen from halides such as chlorides, fluorides, iodides and bromides, carbonates, sulfates, phosphates, nitrates, perchlorates, hydrates thereof, and mixtures thereof.

Metal (Poly)(Hydroxy)(C₁-C₆)Alkyl Carboxylate Crosslinking Agents

According to a more particular embodiment, the crosslinking agent R is an organic metal salt derived from a carboxylic acid.

More particularly, the crosslinking agent R is an organic metal salt chosen from metal (poly)(hydroxy)(C₁-C₆)alkylcarboxylates of alkali salts, alkaline-earth salts, transition metals, and post-transition metals such as aluminium.

It is understood that the metal (poly)(hydroxy)(C₁-C₆)alkylcarboxylate means that the (C₁-C₆)alkyl group is optionally substituted with one or more hydroxyl groups and one or more carboxyl or carboxylate groups. Preferably, the metal (poly)(hydroxy)(C₁-C₆)alkylcarboxylate represents R^{a}—C(O)—OM with M representing a transition metal such as titanium (Ti), or else a post-transition metal such as aluminium (Al), and R^{a} represents a linear or branched (C₁-C₆)alkyl group optionally substituted with at least one hydroxyl group.

According to a preferred embodiment of the invention, the metal salt(s) are organic, preferably chosen from citrates, lactates, glycolates, gluconates, acetates, propionates, fumarates, oxalates, glycinates and tartrates, hydrates thereof, and mixtures thereof, more preferentially acetates, lactates or mixtures thereof such as aluminium acetate or aluminium lactate.

According to a preferred embodiment, the metal salt(s) are chosen from basic aluminium acetate, aluminium oxalate, hydrated or non-hydrated aluminium citrate, aluminium lactate and aluminium glycinate, and mixtures thereof.

According to an even more preferred embodiment, the metal salt is basic aluminium acetate.

### Metal Alkoxide Crosslinking Agents

According to another particular embodiment, the crosslinking agent R is a compound chosen from the metal alkoxides of formulae (XXIVₐ), (XXIV_{b}), (XXIV_{c}) and (XXIV_{d}) below and mixtures thereof:

M-(OR₁)ₙ (XXIVₐ)

R-M-(OR₁)ₙ₋₁ (XXIV_{b})

(R₁O)ₙ₋₁-M-R″-M′-(OR₁′)_{n′-1} (XXIV_{c})

R-M(R′)—(OR₁)ₙ₋₂ (XXIV_{d})

in which formulae (XXIVₐ), (XXIV_{b}), (XXIV_{c}) and (XXIVₐ):
M and M′, which may be identical or different, represent an atom chosen from alkaline-earth metals, transition metals, metals of the lanthanide family, post-transition metals such as aluminium or tin and metalloids such as boron; preferably transition metals such as Ti and post-transition metals such as aluminium;
n and n′ respectively represent the valencies of the atoms represented by M and M′;
R₁ and R₁′, which may be identical or different, represent a linear or branched, saturated or unsaturated hydrocarbon-based group containing from 1 to 30 carbon atoms, preferably from 1 to 6 carbon atoms, said hydrocarbon-based group being optionally interrupted with 1 to 20 heteroatoms chosen from O, N, S and P, notably O or N; and/or said hydrocarbon-based group being optionally substituted with one or more hydroxyl or carbonyl groups;
R and R′, which may be identical or different, represent a hydrogen atom or a linear, branched or cyclic, saturated or unsaturated hydrocarbon-based group containing from 1 to 30 carbon atoms, preferably from 2 to 20 carbon atoms, optionally interrupted with 1 to 20 heteroatoms chosen from O, N, S and/or P, notably O or N, and/or said hydrocarbon-based group being optionally substituted with one or more hydroxyl or carbonyl groups;
R″ represents —O—, —NR₂—, —S— or a linear, cyclic or branched, saturated or unsaturated divalent hydrocarbon-based group containing from 1 to 30 carbon atoms, preferably from 2 to 20 carbon atoms, optionally interrupted with 1 to 20 heteroatoms chosen from O, N, S and P, notably O or N, with R₂ representing a linear, cyclic or branched, saturated or unsaturated hydrocarbon-based group containing from 1 to 30 carbon atoms, preferably from 2 to 20 carbon atoms;

Preferably, M and M′, which may be identical or different, represent an atom chosen from transition metals such as titanium or zirconium or alkaline-earth metals such as magnesium, more preferentially chosen from transition metals such as titanium or zirconium, even more preferentially titanium.

Preferably, the organometallic compound(s) are chosen from the alkoxides of formula (XXIVₐ) as defined previously.

According to a preferred embodiment, the organometallic compound(s) are chosen from the alkoxides of formula (XXIVₐ), in which:
M represents an atom chosen from transition metals, metals of the lanthanide family, post-transition metals such as aluminium, tin, metalloids such as boron, or alkaline-earth metals such as magnesium or calcium;
n represents the valency of the atom represented by M;
R₁ represents a linear or branched saturated hydrocarbon-based group containing from 1 to 30 carbon atoms, preferably from 1 to 6 carbon atoms.

According to a more preferred embodiment, the organometallic compounds are chosen from the alkoxides of formula (XXIVₐ) in which:
M represents an atom chosen from transition metals such as zirconium or titanium, metals of the lanthanide family, post-transition metals such as aluminium, tin, metalloids such as boron, and alkaline-earth metals such as magnesium, preferably M represents a titanium atom;
n represents the valency of the atom represented by M, notably 1, 2, 3 or 4, in particular 4;
R₁ represents a methyl, ethyl, 2-ethylhexyl, propyl, isopropyl, n-butyl, isobutyl or t-butyl group.

According to an even more preferred embodiment, the organometallic compound(s) are chosen from zirconium ethoxide (Zr(OC₂H₅)₄), zirconium propoxide (Zr(OCH₂CH₂CH₃)₄), zirconium isopropoxide (Zr(OCH(CH₃)₂)₄), zirconium butoxide Zr(OCH₂CH₂CH₂CH₃)₄, zirconium tert-butoxide (Zr(OC(CH₃)₃)₄), titanium ethoxide (Ti(OC₂H₅)₄), titanium propoxide (Ti(OCH₂CH₂CH₃)₄), titanium isopropoxide (Ti(OCH(CH₃)₂)₄), titanium butoxide (Ti(OCH₂CH₂CH₂CH₃)₄), titanium tert-butoxide (Ti(OC(CH₃)₃)₄), titanium 2-ethylhexyloxide (Ti(OCH₂CH(C₂H₅)(CH₂)₃CH₃)₄), and mixtures thereof.

Particularly preferably, the organometallic compound(s) are chosen from zirconium propoxide (Zr(OCH₂CH₂CH₃)₄), titanium propoxide (Ti(OCH₂CH₂CH₃)₄), titanium butoxide (Ti(OCH₂CH₂CH₂CH₃)₄) and mixtures thereof.

More preferably, the crosslinking agent R is a compound of formula (XXIVₐ) preferably in which M represents an atom chosen from transition metals, notably titanium such as titanium butoxide.

### Crosslinking Agents Composed of a Metal Belonging to the Group of the Rare-Earth Metals

According to another particular embodiment, the crosslinking agent R is a compound of a metal belonging to the group of the rare-earth metals M″, and notably a salt of a metal belonging to the group of the rare-earth metals.

The term “salt of a metal belonging to the group of the rare-earth metals” means a salt notably derived from the action of an acid on a metal belonging to the group of the rare-earth metals.

The compound(s) of a metal belonging to the group of the rare-earth metals may be in the form of hydrates.

The compound(s) of a metal belonging to the group of the rare-earth metals may be organic or mineral. They may or may not be in salt form.

The term “organic salt of a metal belonging to the rare-earth metal group” means a salt notably derived from the action of an organic acid (notably a carboxylic acid) on a metal belonging to the group of the rare-earth metals.

The term “mineral salt of a metal belonging to the group of the rare-earth metals” means a salt notably derived from the action of a mineral acid on a metal belonging to the group of the rare-earth metals.

The term “inorganic acid” means an acid which does not include any carbon atoms, apart from carbonic acid.

As examples of metals belonging to the group of the rare-earth metals M″, mention may be made of scandium, yttrium, lanthanum, cerium, praseodymium, neodymium, promethium, samarium, europium, gadolinium, terbium, dysprosium, holmium, erbium, thulium, ytterbium and lutetium. Preferably, the metal(s) belonging to the group of the rare-earth metals are chosen from cerium, yttrium, ytterbium, lanthanum and europium; more preferentially, the metal(s) belonging to the group of the rare-earth metals M″ are chosen from cerium and yttrium.

Preferably, the metal belonging to the group of the rare-earth metals M″ is chosen from cerium, yttrium, ytterbium, lanthanum and europium, and mixtures thereof. More preferentially, the metal belonging to the group of the rare-earth metals is chosen from cerium and yttrium, and mixtures thereof.

Preferably, the metal belonging to the group of the rare-earth metals M″ is in the oxidation state +III.

According to the invention, the compound of a metal belonging to the group of the rare-earth metals is chosen from rare-earth metal salts and rare-earth metal complexes.

The term “rare-earth metal complex” refers to the combination of the metal M″ with one or more ligands.

In the text hereinbelow, the term “ligand” refers to an ion or a molecule bearing a group which combines, via an ionic bond and/or a coordination bond, with the metal M″. The same ligand may bear several groups which combine via an ionic bond and/or a coordination bond.

A definition of rare-earth metal salts or complexes may be found in: Progress in the Science and Technology of the Rare Earths, Volume 1, edited by Leroy Eyring in 1964, published by Macmillan Company and written by F. Gaume-Mahn, page 259 etseq.

The rare-earth metal salts and complexes according to the invention are characterized in that they contain at least one metal atom M″ belonging to the group of the rare-earth metals and that said atom is in the +III oxidation state.

The metal belonging to the group of the rare-earth metals M″ may then be associated, via its electron shell, with n1 anionic groups forming an ionic bond with M″ and/or with n2 groups forming a coordination bond with M″. The groups forming a coordination bond are, for example, groups with a donor doublet, such as carbonyl or amine.

If n2=0, the compound of the metal belonging to the group of the rare-earth metals forms a salt and, in this case, the metal M″ belonging to the group of the rare-earth metals is associated with three anionic groups.

If n2>0, the compound of the metal belonging to the group of the rare-earth metals forms a complex and, in this case, the number of anionic groups n1 may range from 0 to 3.

The metal M″ belonging to the group of the rare-earth metals is associated with one or more anionic groups and/or one or more groups forming a coordination bond.

The ligands associated with the metals belonging to the group of the rare-earth metals M″ to form a corresponding rare-earth metal complex are as described below.

a) Typically, the ligand may be a monoanionic ion, which may or may not be monoatomic, such as a nitrate, or a hydroxyl (OH—) or a halide (typically chloride or bromide). By way of example, the resulting rare-earth metal compound may then be M″Cl₃, M″(OH)₃ or M″(NO₃)₃, and in particular CeNO₃, YNO₃, LaNO₃, CeCl₃, YCl₃, LaCl₃, more preferentially rare-earth metal halides, notably Ce and Y halides such as CeCl₃ and YCl₃.

b) The ligand may be a dianionic or trianionic ion, such as phosphate or sulfate. By way of example, mention may be made of rare-earth metal compounds such as MPO₄, or M₂(SO₄)₃ and in particular CePO₄, YPO₄, LaPO₄, Ce₂(SO₄)₃, Y₂(SO₄)₃ and La₂(SO₄)₃.

c) The ligand may contain one or more groups forming a coordination bond and a function forming an ionic bond.

Thus, the ligand may be a monocarboxylate or polycarboxylate molecule, such as acetate or succinate. In this case, it is considered that the carboxylate function acts as an anionic group, by means of the hydroxyl of the carboxylic group, and acts as a group forming a coordination bond by means of the lone pair of the oxygen of the carbonyl function. Thus, the resulting rare-earth metal compound may be M″(R—(COO)ₙ)_{3/n}. In addition to bearing one or more carboxylates, the ligand may include other functions, such as hydroxyls or amines. Thus, the ligand may consist totally or partially of hydroxycarboxylic acids or aminocarboxylic acids. As monocarboxylic or polycarboxylic compound bearing additional functions, mention may be made of tartrate, citrate, glycolate or ethylenediaminetetraacetate (EDTA) ions.

The ligand may bear a non-localized anionic charge, for instance acetylacetonate. The rare-earth metal compound will then be M″(acetylacetonate)₃ or M″(acetylacetonate)₃·7H₂O in which each acetonate bonds to the metal M″ via its two carbonyl functions, one acting as an anionic group, the other as a group bonding by coordination.

The ligand may also be of the aromatic type, such as a phenol, a cyclopentadiene (Progress in the Science and Technology of the Rare Earths, published by Leroy Eyring and written by F. Gaume-Mahn, page 296), or a pyridine.

d) The rare-earth metal compound may include one or more ligands forming a coordination bond and one or more ligands forming an ionic bond. Thus, the rare-earth metal compound may be yttrium dihydroxyacetate Y((OH)₂acetate) (Synthesis and Properties of Yttrium Hydroxyacetate Sols by S. S. Balabanov, E. M. Gavrishchuk, and D. A. Permin, Inorganic Materials, 2012, Vol. 48, No. 5, pages 500-503).

e) The rare-earth metal compound may be a mixed salt in which one of the cations M′″ represents a cation other than a rare-earth metal cation, such as an alkali or alkaline-earth or a cationic organic cation, notably a quaternary amine (or ammonium), for example mono/di/tri/tetra(C₁-C₄)alkylammonium, or mono/di(C₁-C₄)alkyl imidazolium, (C₁-C₄)alkylpyridinium; more particularly, the mixed salt rare-earth metal compound is Li,Ce(SO₄)₂.

The compounds belonging to the group of the rare-earth metals, which are often highly hygroscopic, may be in the form of hydrates, for instance CeCl₃·7H₂O, YCl₃·6H₂O, LaCl₃·7H₂O or Ce(acetonate)₃·xH₂O.

According to a particular embodiment of the invention, the compound(s) belonging to the group of the rare-earth metals are chosen from the salts of organic acids such as citrates, lactates, glycolates, gluconates, acetates, propionates, fumarates, oxalates, tartrates, mesylates and methosulfates, notably gluconates, hydrates thereof, and mixtures thereof.

According to a preferred embodiment, the salt(s) of a metal belonging to the group of the rare-earth metals are mineral salts.

Preferably, the mineral salt(s) of a metal belonging to the group of the rare-earth metals are chosen from halides such as chlorides, fluorides, iodides and bromides, carbonates, sulfates, phosphates, nitrates and perchlorates, hydrates thereof, and mixtures thereof.

More preferentially, the mineral salt(s) of a metal belonging to the group of the rare-earth metals are chosen from halides such as chlorides, fluorides, iodides and bromides, and nitrates, hydrates thereof, and mixtures thereof.

Even more preferentially, the mineral salt(s) of a metal belonging to the group of the rare-earth metals are chosen from chlorides and nitrates, hydrates thereof, and mixtures thereof.

According to a particularly preferred embodiment, the compound(s) belonging to the group of the rare-earth metals are chosen from Ce(NO₃)₃, Y(NO₃)₃, La(NO₃)₃, CeCl₃, YCl₃ and LaCl₃, and mixtures thereof.

According to an even more preferred embodiment, the compound(s) belonging to the group of the rare-earth metals are chosen from CeCl₃ and YCl₃, and mixtures thereof.

According to a preferred embodiment, the crosslinking agent is chosen from (poly)amino, (poly)thiolated and/or (poly)hydroxylated, (poly)carbonyl and (poly)acrylate compounds, and mixtures thereof, and preferably from (poly)amino and (poly)thiolated compounds, notably with said (poly)amino being chosen from chitosans, aminoalkoxysilanes and polydimethylsiloxanes comprising primary amine groups at the end of the chain or on side chains, and even more preferentially chosen from poly(D-glucosamine), 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane and polydimethylsiloxanes comprising aminopropyl end groups at the end of the chain, and even more preferentially 3-aminopropyltriethoxysilane (APTES), and notably said (poly)thiolated compound being chosen from polydiorganosiloxanes bearing thiol functions, aminosilicones bearing thiol functions, alkoxysilanes bearing thiol functions, organic polythiols, natural products modified with thiol groups, amino thiols derived from dendrimers or polyethyleneimines (PEI) and silicone thiols, the (poly)thiolated compound preferably being chosen from polydimethylsiloxanes terminated with mercaptopropyl groups and dimethicone/mercaptopropyl methicone copolymers.

According to a preferred embodiment, the crosslinking agent is chosen from i) (poly)amine compounds, ii) (poly)thiolated and/or (poly)hydroxylated compounds, iii) (poly)carbonylated compounds such as terephthaldehyde, iv) (poly)acrylates such as trimethylolpropane triacrylate, v) metal salts chosen from vₐ) metal alkoxides such as titanium butoxide, v_{b}) metal (poly)(hydroxy)(C₁-C₆)alkylcarboxylates, notably of transition metals or of post-transition metals, notably aluminium, such as aluminium acetate or aluminium lactate and v_{c}) salts of metals belonging to the group of the rare-earth metals, in particular Ce or Y halides such as CeCl₃ and YCl₃, and vi) mixtures thereof, and preferably chosen from (poly)amino and (poly)thiolated compounds, with said (poly)amino notably being chosen from chitosans, aminoalkoxysilanes and polydimethylsiloxanes comprising primary amine groups at the end of the chain or on the side chains, and even more preferentially chosen from poly(D-glucosamine), 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane and polydimethylsiloxanes containing aminopropyl end groups at the end of the chain, and even more preferably 3-aminopropyltriethoxysilane (APTES), and notably said (poly)thiolated compound being chosen from polydiorganosiloxanes bearing thiol functions, aminosilicones bearing thiol functions, alkoxysilanes bearing thiol functions, organic polythiols, natural products modified with thiol groups, amino thiols derived from dendrimers or polyethyleneimines (PEI) and silicone thiols, the (poly)thiolated compound preferably being chosen from polydimethylsiloxanes terminated with mercaptopropyl groups and dimethicone/mercaptopropyl methicone copolymers.

The mixtures vi) may refer to mixtures of compounds of the same type, for instance mixtures of (poly)amines i) of different structures, or mixtures of (poly)thiols ii) of different structures. The mixtures vi) may also refer to mixtures of compounds of different types, for instance mixtures consisting of one or more polyamines i) with one or more (poly)thiols ii).

According to another particular embodiment, the crosslinking agent R is a compound chosen from vₐ) a (C₁-C₆)alkoxide of a transition metal, notably of titanium, such as titanium butoxide, and v_{b}) a (poly)(hydroxy)(C₁-C₆)alkylcarboxylate of a transition metal, notably of aluminium, such as aluminium acetate, or aluminium lactate.

According to another particular embodiment, the crosslinking agent R is a compound chosen from metal alkoxides of formula (XXIVₐ) as defined previously, preferably in which the metal M is a transition metal, notably of titanium such as titanium butoxide.

According to another particular embodiment, the crosslinking agent R is a compound chosen from iii) (poly)carbonyls such as terephthaldehyde or trimethylolpropane triacrylate.

More preferentially, the crosslinking agents of the invention are chosen from amine polymers, thiolated polymers and (poly)acrylate, preferably silicone polymers such as amodimethicones of formula (III′b) as defined previously; and

the polythiolated compounds are chosen from those of formula (XVb) as defined previously and more preferentially of formula (XVb′) as defined previously.

According to a particular embodiment of the invention, the composition comprises c) one or more fatty substances.

### c) The Fatty Substances

The composition may also comprise one or more fatty substances.

The term “fatty substance” means an organic compound that is insoluble in water at ordinary room temperature (25° C.) and at atmospheric pressure (760 mmHg) (solubility of less than 5%, preferably 1% and even more preferentially 0.1%). They bear in their structure at least one hydrocarbon-based chain including at least 6 carbon atoms or a sequence of at least two siloxane groups. In addition, the fatty substances are generally soluble in organic solvents under the same temperature and pressure conditions, for instance chloroform, ethanol, benzene, liquid petroleum jelly or decamethylcyclopentasiloxane.

The fatty substance(s) of the invention are of natural or synthetic origin, preferably natural, more preferentially of plant origin. These fatty substances are preferably neither polyoxyethylenated nor polyglycerolated. They are different from fatty acids since salified fatty acids constitute soaps which are generally soluble in aqueous media.

According to a particular embodiment of the invention, the composition comprises one or more fatty substances that are not liquid at 25° C. and at atmospheric pressure.

### The Wax(es)

According to a particular embodiment, the composition of the invention comprises one or more waxes.

The term “wax” means a lipophilic compound that is solid at room temperature (25° C.), with a reversible solid/liquid change of state, having a melting point of greater than or equal to 30° C., which may be up to 200° C. and notably up to 120° C.

In particular, the wax(es) that are suitable for use in the invention may have a melting point of greater than or equal to 45° C. and in particular of greater than or equal to 55° C.

The composition according to the invention preferably comprises a content of wax(es) ranging from 3% to 20% by weight relative to the total weight of the composition, in particular from 5% to 15% and more particularly from 6% to 15%.

According to a particular form of the invention, the composition of the invention is solid, in particular anhydrous. It may then be in stick form; use will be made of polyethylene microwaxes in the form of crystallites with an aspect ratio at least equal to 2, and with a melting point ranging from 70 to 110° C. and preferably from 70 to 100° C., so as to reduce or even eliminate the presence of strata in the solid composition. These crystallites in needle form and notably the dimensions thereof may be characterized visually according to the following method.

### The Pasty Compound(s)

According to a particular embodiment, the composition of the invention comprises one or more pasty compounds.

For the purposes of the present invention, the term “pasty compound” means a lipophilic fatty compound that undergoes a reversible solid/liquid change of state, having anisotropic crystal organization in the solid state, and including, at a temperature of 23° C., a liquid fraction and a solid fraction.

According to a preferred embodiment of the invention, the composition comprises one or more fatty substances that are liquid at 25° C. and at atmospheric pressure, which are particularly hydrocarbon-based.

The hydrocarbon-based liquid fatty substance(s) are notably chosen from C₆-C₁₆ hydrocarbons or hydrocarbons comprising more than 16 carbon atoms and up to 60 carbon atoms, preferably between C₆ and C₁₆, and in particular alkanes, oils of animal origin, oils of plant origin, glycerides or fluoro oils of synthetic origin, fatty alcohols, fatty acid and/or fatty alcohol esters, and silicones. In particular, the liquid fatty substance(s) are chosen from non-silicone oils.

It is recalled that, for the purposes of the invention, the fatty alcohols, fatty esters and fatty acids more particularly contain one or more linear or branched, saturated or unsaturated hydrocarbon-based groups comprising 6 to 60 carbon atoms, which are optionally substituted, in particular with one or more hydroxyl groups OH (in particular from 1 to 4 hydroxyl groups). If they are unsaturated, these compounds may comprise one to three unsaturations, preferably from one to three conjugated or unconjugated carbon-carbon double bonds.

As regards the C₆-C₁₆ alkanes, these compounds are linear or branched, and optionally cyclic; preferably, the fatty substance(s) c) of the invention are chosen from linear or branched C₈-C₁₄, more preferentially C₉-C₁₃ and even more preferentially C₉-C₁₂ alkanes. Examples that may be mentioned include hexane, undecane, dodecane, tridecane, and isoparaffins, for instance isohexadecane, isododecane or isodecane. The linear or branched hydrocarbons containing more than 16 carbon atoms may be chosen from liquid paraffins, liquid petroleum jelly, polydecenes, and hydrogenated polyisobutene such as Parleam®.

Among the hydrocarbon-based liquid fatty substances c) having an overall solubility parameter according to the Hansen solubility space of less than or equal to 20 (MPa)¹¹², mention may be made of oils, which may be chosen from natural or synthetic, hydrocarbon-based oils, which are optionally fluorinated and optionally branched, alone or as a mixture.

According to a very advantageous embodiment, the composition of the invention comprises one or more fatty substances which are one or more hydrocarbon-based oils. The hydrocarbon-based oil(s) may be volatile or non-volatile.

According to a preferred embodiment of the invention, the fatty substance(s) c) are linear or branched hydrocarbon-based oils, which are volatile, notably chosen from undecane, dodecane, isododecane, tridecane, and a mixture of various volatile oils thereof preferably comprising isododecane in the mixture, or a mixture of undecane and tridecane.

According to another particular embodiment, the liquid fatty substance(s) c) are a mixture of a volatile hydrocarbon-based oil and a non-volatile hydrocarbon-based oil, the mixture of which preferentially comprises dodecane or isododecane as volatile oil.

In particular, the fatty substance(s) c) of the invention are a mixture of C₉-C₁₂ alkanes, preferably of natural origin, the chains of which comprise from 9 to 12 carbon atoms, preferably linear or branched C₉-C₁₂ alkanes. This mixture is notably known under the INCI name C₉-C₁₂ Alkane E511470, CAS 68608-12-8, Vegelight Silk® sold by BioSynthls. This biodegradable mixture of volatile oils is obtained from coconut oil (the viscosity is 0.9-1.1 cSt (40° C.) and it has a flash point of 65° C.).

Volatile silicone oils that may be mentioned include volatile linear or cyclic silicone oils, notably those with a viscosity of less than or equal to 8 centistokes (cSt) (8×10⁻⁶ m²/s), and notably containing from 2 to 10 silicon atoms and in particular from 2 to 7 silicon atoms, these silicones optionally including alkyl or alkoxy groups containing from 1 to 10 carbon atoms. As volatile silicone oils that may be used in the invention, mention may notably be made of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

As non-volatile silicone oils, mention may be made of linear or cyclic non-volatile polydimethylsiloxanes (PDMSs); polydimethylsiloxanes including alkyl, alkoxy and/or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes, 2-phenylethyl trimethylsiloxysilicates and pentaphenyl silicone oils.

The hydrocarbon-based oil may be chosen from:
hydrocarbon-based oils containing from 8 to 14 carbon atoms, and notably:
branched C₈-C₁₄ alkanes, for instance C₈-C₁₄ isoalkanes of petroleum origin (also known as isoparaffins), for instance isododecane (also known as 2,2,4,4,6-pentamethylheptane), isodecane and, for example, the oils sold under the trade names Isopar or Permethyl,
linear alkanes, for instance n-dodecane (C12) and n-tetradecane (C14) sold by Sasol under the respective references Parafol 12-97 and Parafol 14-97, and also mixtures thereof, the undecane-tridecane mixture, the mixtures of n-undecane (C11) and of n-tridecane (C13) obtained in examples 1 and 2 of patent application WO 2008/155059 from the company Cognis, and mixtures thereof, and also mixtures of n-undecane (C11) and of n-tridecane (C13) Cetiol Ultimate® from the company BASF,
short-chain esters (containing from 3 to 8 carbon atoms in total) such as ethyl acetate, methyl acetate, propyl acetate or n-butyl acetate,
hydrocarbon-based oils of plant origin such as triglycerides consisting of fatty acid esters of glycerol, the fatty acids of which may have various chain lengths ranging from C₄ to C₂₄, these chains possibly being linear or branched, and saturated or unsaturated; these oils are notably heptanoic acid or octanoic acid triglycerides, or alternatively wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cotton oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion flower oil, musk rose oil or coconut oil; shea butter; or else caprylic/capric acid triglycerides, for instance those sold by the company Stearinerie Dubois or those sold under the names Miglyol 810*, 812* and 818* by the company Dynamit Nobel,
synthetic ethers containing from 10 to 40 carbon atoms,
linear or branched hydrocarbons of mineral or synthetic origin, such as petroleum jelly, polydecenes, hydrogenated polyisobutene such as Parleam®, squalane and liquid paraffins, and mixtures thereof,
esters such as the oils of formula R¹C(O)—O—R² in which R¹ represents a linear or branched fatty acid residue including from 1 to 40 carbon atoms and R² represents a, notably branched, hydrocarbon-based chain containing from 1 to 40 carbon atoms, on condition that R¹+R²≥10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, 2-hexyldecyl laurate, 2-octyldecyl palmitate, 2-octyldodecyl myristate, alcohol or polyalcohol heptanoates, octanoates, decanoates or ricinoleates such as propylene glycol dioctanoate; hydroxylated esters such as isostearyl lactate, diisostearyl malate and 2-octyldodecyl lactate; polyol esters and pentaerythritol esters, more preferentially esters of a linear or branched C₈-C₁₀ fatty acid and of a linear or branched C₁₂-C₁₈ fatty alcohol alone or as a mixture with alkanes derived from the complete hydrogenation/reduction of fatty acids obtained from Cocos nucifera (coconut) oil, particularly dodecane or mixtures of cocoyl caprylate/caprate with dodecane; mention may be made of those having the INCI name Coconut alkanes (and) cococaprylate/caprate sold under the name Vegelight 1212LC® by Grant Industries,
fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2-undecylpentadecanol.

In addition to the hydrocarbon-based liquid fatty substance, the composition of the invention may comprise a silicone oil. If silicone oil is in the composition of the invention, it is preferably in an amount which does not exceed 10% by weight relative to the weight of the composition, more particularly in an amount of less than 5% and more preferentially less than 2% by weight relative to the total weight of the composition.

In particular, the composition comprises at least one hydrocarbon-based liquid fatty substance c) chosen from:
plant oils formed by fatty acid esters of polyols, in particular triglycerides, such as sunflower oil, sesame oil, rapeseed oil, macadamia oil, soybean oil, sweet almond oil, beauty-leaf oil, palm oil, grapeseed oil, corn oil, arara oil, cottonseed oil, apricot oil, avocado oil, jojoba oil, olive oil, coconut oil or cereal germ oil;
linear, branched or cyclic esters containing more than 6 carbon atoms, notably 6 to 30 carbon atoms; and notably isononyl isononanoate;
   and more particularly esters of formula R^{d}—C(O)—O—R^{e} in which R^{d} represents a higher fatty acid residue including from 7 to 19 carbon atoms and R^{e} represents a hydrocarbon-based chain including from 3 to 20 carbon atoms, such as palmitates, adipates, myristates and benzoates, notably diisopropyl adipate and isopropyl myristate; more preferentially esters of formula R^{d}—C(O)—O—R^{e} in which R^{d} represents a higher fatty acid residue including from 8 to 10 carbon atoms and R^{e} represents a hydrocarbon-based chain including from 12 to 18 carbon atoms:
hydrocarbons and notably volatile or non-volatile linear, branched and/or cyclic alkanes, such as optionally volatile C₅-C₆₀ isoparaffins, such as undecane, dodecane, isododecane, tridecane, Parleam (hydrogenated polyisobutene), isohexadecane, cyclohexane, or Isopars, and mixtures thereof; or alkanes resulting from the complete hydrogenation/reduction of mixtures of fatty acids derived from Cocos nucifera (coconut) oil, such as dodecane, the mixture of C₉-C₁₂ alkanes, the chains of which comprise from 9 to 12 carbon atoms, preferably linear or branched C₉-C₁₂ alkanes, in particular comprising dodecane, or else liquid paraffin, liquid petroleum jelly, or hydrogenated polyisobutylene;
ethers containing 6 to 30 carbon atoms;
ketones containing 6 to 30 carbon atoms;
aliphatic fatty monoalcohols containing 6 to 30 carbon atoms, the hydrocarbon-based chain not including any substitution groups, such as oleyl alcohol, decanol, dodecanol, octadecanol, octyldodecanol and linoleyl alcohol;
polyols containing 6 to 30 carbon atoms, such as hexylene glycol; and
mixtures thereof, such as mixtures of esters of linear or branched C₈-C₁₀ fatty acid and C₁₂-C₁₈ fatty alcohol and alkanes resulting from the complete hydrogenation/reduction of mixtures of fatty acids from Cocos nucifera (coconut) oil, in particular dodecane, such as mixtures of cococaprylate/caprate and dodecane; mention may be made of those having the INCI name Coconut alkanes (and) coco-caprylate/caprate sold under the name Vegelight 1212LC® by Grant Industries; or mixtures of C₉-C₁₂ alkanes, the chains of which comprise from 9 to 12 carbon atoms, preferably linear or branched C₉-C₁₂ alkanes, in particular comprising dodecane; mention may be made of the oil mixture having the INCI name C₉₋₁₂ Alkane, Vegelight Silk® sold by BioSynthls.

Preferably, the composition of the invention comprises at least one hydrocarbon-based liquid fatty substance c) chosen from:
plant oils formed by fatty acid esters of polyols, in particular triglycerides,
esters of formula R^{d}—C(O)—O—R^{e} in which R^{d} represents a higher fatty acid residue including from 7 to 19 carbon atoms and R^{e} represents a hydrocarbon-based chain including from 3 to 20 carbon atoms, more preferentially esters of formula R^{d}—C(O)—O—R^{e} in which R^{d} represents a higher fatty acid residue including from 8 to 10 carbon atoms and R^{e} represents a hydrocarbon-based chain including from 12 to 18 carbon atoms;
volatile or non-volatile, linear or branched C₈-C₆₀ alkanes, such as isododecane and alkanes resulting from the complete hydrogenation/reduction of mixtures of fatty acids obtained from Cocos nucifera (coconut) oil, in particular dodecane;
volatile or non-volatile, non-aromatic cyclic C₅-C₁₂ alkanes;
ethers containing 7 to 30 carbon atoms;
ketones containing 8 to 30 carbon atoms;
aliphatic fatty monoalcohols containing 12 to 30 carbon atoms, the hydrocarbon-based chain not including any substitution groups, and
mixtures thereof.

Advantageously, the fatty substance(s) c) of the invention, which are notably liquid, are apolar, i.e. formed solely of carbon and hydrogen atoms.

The hydrocarbon-based liquid fatty substance(s) are preferably chosen from hydrocarbon-based oils containing from 8 to 14 carbon atoms, which are in particular volatile, more particularly the apolar oils described previously.

Preferentially, the fatty substance(s) c) of the invention, which are notably liquid, are chosen from alkanes such as dodecane, isododecane, fatty alcohols such as octyldodecanol, esters such as isononyl isononanoate, cocoyl caprylate/caprate and mixtures thereof, more preferentially alkanes.

More particularly, the fatty substance(s) c) of the invention, which are notably liquid, are chosen from linear or branched C₆-C₁₆, preferably C₈-C₁₄, more preferentially C₉-C₁₃ and even more preferentially C₉-C₁₂ alkanes, and even more preferentially the alkanes are volatile. More particularly, the liquid fatty substance(s) c) of the invention are volatile and are chosen from undecane, dodecane, isododecane, tridecane, and a mixture thereof notably comprising dodecane, isododecane or a mixture of undecane and tridecane.

Preferentially, the liquid fatty substance(s) c) of the invention, which are notably liquid, are isododecane.

According to another advantageous embodiment of the invention, the fatty substance(s) c) of the invention, which are notably liquid, are a mixture of non-volatile oil(s) and volatile oil(s); preferably, the mixture comprises, as volatile oil, undecane, dodecane, isododecane or tridecane, more preferentially isododecane. A mixture of volatile oil and non-volatile oil that may be mentioned is the mixture of isododecane and of isononyl isononanoate.

More preferentially, when the fatty substance(s) are a mixture of volatile oil and of non-volatile oil, the amount of volatile oil is greater than the amount of non-volatile oil.

In particular, in the mixture, the non-volatile oil is a phenyl silicone oil, preferably chosen from pentaphenyl silicone oils.

Advantageously, the composition comprises one or more fatty substances, which are notably liquid at 25° C. and at atmospheric pressure, preferably one or more oils, of the fatty medium in a content ranging from 2% to 99.9% by weight, relative to the total weight of the composition, preferably ranging from 5% to 90% by weight, preferably ranging from 10% to 80% by weight, preferably ranging from 20% to 80% by weight.

### (d) Water

According to a particular embodiment of the invention, the composition also comprises water.

According to one embodiment, the composition of the invention predominantly comprises on a weight basis the weight amount of fatty substances versus the weight amount of water.

More particularly, the composition of the invention comprises an amount of water of less than or equal to 5% by weight relative to the total weight of the composition, preferentially less than or equal to 2% by weight, more preferentially less than 1% by weight, relative to the total weight of the composition; even more preferentially, the composition of the invention is anhydrous, i.e. it does not comprise any water.

e) Additional solvents

According to a particular embodiment of the invention, the composition comprises one or more solvents other than c), which are preferably polar and/or protic, other than water in the predominantly fatty medium.

The solvent(s), which are preferably polar and/or protic, other than water are present in the composition in a weight percentage of between 0% and 10% relative to the total weight of the solvent mixture, preferentially between 0.5% and 8%, more particularly between 1% and 5%, such as 2% by weight, relative to the total weight of the composition.

Preferably, the composition of the invention comprises one or more solvents, particularly polar protic solvents such as alkanols, more preferentially C₂-C₆ alkanols, such as ethanol.

### Form of the Composition:

According to one embodiment of the invention, the composition comprises an aqueous phase. The composition is notably formulated as aqueous lotions or as water-in-oil or oil-in-water emulsions or as multiple emulsions (oil-in-water-in-oil or water-in-oil-in-water triple emulsions (such emulsions are known and described, for example, by C. Fox in “Cosmetics and Toiletries”—November 1986—Vol. 101—pages 101-112)).

According to a particular embodiment of the invention, the composition is a direct emulsion, i.e. an emulsion of oil-in-water or O/W type. The weight amount of oil is preferably less than 40% in the inverse emulsion, preferably less than or equal to 35%, more particularly less than or equal to 30% by weight relative to the total weight of the composition.

More particularly, in the direct emulsion, the amount of water is greater than or equal to 40% by weight relative to the total weight of the composition, more particularly greater than or equal to 45%, preferentially greater than or equal to 50%.

According to another particular embodiment of the invention, the composition of the invention is an inverse emulsion, i.e. of water-in-oil or W/O type. The weight amount of oil is preferably greater than 30% in the inverse emulsion, preferably greater than 40%, more preferentially greater than or equal to 45% by weight relative to the total weight of the composition. More particularly, in the inverse emulsion, the amount of water is less than 40% by weight relative to the total weight of the composition, preferably less than or equal to 30% by weight, more preferably less than 20% by weight.

The aqueous phase of the composition contains water and in general other water-soluble or water-miscible solvents such as polar and protic solvents as defined below (see additional solvents).

The composition according to the invention preferably has a pH ranging from 3 to 9, depending on the support chosen.

According to a particular embodiment of the invention, the pH of the composition(s) is neutral or even slightly acidic. Preferably, the pH of the composition is between 6 and 7. The pH of these compositions may be adjusted to the desired value by means of acidifying or basifying agents usually used in cosmetics, or alternatively using standard buffer systems.

The term “basifying agent” or “base” means any agent for increasing the pH of the composition in which it is present. The basifying agent is a Brønsted, Lowry or Lewis base. It may be mineral or organic. Particularly, said agent is chosen from a) aqueous ammonia, b) (bi)carbonate, c) alkanolamines such as monoethanolamine, diethanolamine, triethanolamine and derivatives thereof, d) oxyethylenated and/or oxypropylenated ethylenediamines, e) organic amines, f) mineral or organic hydroxides, g) alkali metal silicates such as sodium metasilicates, h) amino acids, preferably basic amino acids such as arginine, lysine, ornithine, citrulline and histidine, and i) the compounds of formula (F) below:

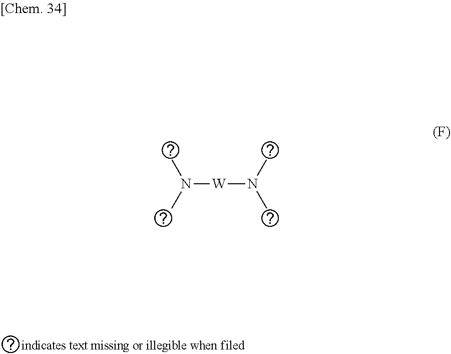

in which formula (F):
W is a divalent C₁-C₆ alkylene radical optionally substituted with one or more hydroxyl groups or a C₁-C₆ alkyl radical, and/or optionally interrupted with one or more heteroatoms such as O or NRᵤ;
Rₓ, R_{y}, R_{z}, Rₜ and Rᵤ, which may be identical or different, represent a hydrogen atom or a C₁-C₆ alkyl, C₁-C₆ hydroxyalkyl or C₁-C₆ aminoalkyl radical.

Examples of amines of formula (F) that may be mentioned include 1,3-diaminopropane, 1,3-diamino-2-propanol, spermine and spermidine.

The term “alkanolamine” means an organic amine comprising a primary, secondary or tertiary amine function, and one or more linear or branched C₁—C alkyl groups bearing one or more hydroxyl radicals.

Among the mineral or organic hydroxides, mention may be made of those chosen from a) hydroxides of an alkali metal, b) hydroxides of an alkaline-earth metal, for instance sodium hydroxide or potassium hydroxide, c) hydroxides of a transition metal, d) hydroxides of lanthanides or actinides, quaternary ammonium hydroxides and guanidinium hydroxide. The mineral or organic hydroxides a) and b) are preferred.

Among the acidifying agents for the compositions used in the invention, examples that may be mentioned include mineral or organic acids, for instance hydrochloric acid, orthophosphoric acid, sulfuric acid, carboxylic acids, for instance acetic acid, tartaric acid, citric acid or lactic acid, or sulfonic acids.

The basifying agents and the acidifying agents as defined previously preferably represent from 0.001% to 20% by weight relative to the weight of the composition containing them and more particularly from 0.005% to 8% by weight of the composition.

According to a preferred embodiment of the invention, the composition comprises an amount of water of less than or equal to 10% by weight relative to the total weight of the composition. Even more preferentially, the composition comprises an amount of water of less than or equal to 5%, better still less than 2%, even better still less than 0.5%, and is notably free of water. Where appropriate, such small amounts of water may notably be introduced by ingredients of the composition that may contain residual amounts thereof.

The composition according to the invention may be in the form of an anhydrous composition.

The term “anhydrous composition” means a composition containing less than 2% by weight of water, or even less than 0.5% of water, and is notably free of water.

According to a particular embodiment of the invention, the composition does not comprise any water.

Advantageously, the composition according to the invention comprises a physiologically acceptable medium. In particular, the composition is a cosmetic composition.

The term “physiologically acceptable medium” means a medium that is compatible with human keratin materials, for instance the skin, the lips, the nails, the eyelashes, the eyebrows or the hair.

The term “cosmetic composition” means a composition that is compatible with keratin materials, which has a pleasant colour, odour and feel and which does not cause any unacceptable discomfort (stinging, tautness or redness) liable to discourage the consumer from using it.

The term “keratin materials” means the skin (body, face, contour of the eyes, scalp), head hair, the eyelashes, the eyebrows, bodily hair, the nails or the lips.

### f) the Adjuvants

The composition according to the invention may comprise one or more cosmetic additives chosen from: i) fragrances, ii) preserving agents, iii) fillers, iv) colouring agents, v) UV-screening agents, vi) ionic or nonionic surfactants, vii) moisturizers, viii) vitamins, ix) ceramides, x) antioxidants, xi) free-radical scavengers, xii) polymers other than PHA a), xiii) thickeners. In particular, the composition according to the invention also comprises iv) one or more colouring agents chosen from pigments, direct dyes and mixtures thereof, preferably pigments; more preferentially, the pigment(s) of the invention are chosen from carbon black, iron oxides, notably black or red iron oxides, and micas coated with iron oxide, triarylmethane pigments, notably blue and violet triarylmethane pigments, such as Blue 1 Lake, azo pigments, notably red azo pigments, such as D&C Red 7, an alkali metal salt of lithol red, such as the calcium salt of lithol red B, even more preferentially red iron oxides.

The composition according to the invention may also comprise one or more fillers, notably in a content ranging from 0.01% to 30% by weight, relative to the total weight of the composition, preferably ranging from 0.01% to 20% by weight. The term “fillers” should be understood as meaning colourless or white, mineral or synthetic particles of any shape, which are insoluble in the medium of the composition, irrespective of the temperature at which the composition is manufactured. These fillers notably serve to modify the rheology or texture of the composition.

Advantageously, the composition according to the invention is a makeup composition, in particular a lip makeup composition, a mascara, an eyeliner, an eye shadow or a foundation.

### Cosmetic Process for Treating Keratin Materials:

According to one form of the invention, the process for treating keratin materials comprises the following steps i) and iii):
i) optionally applying to the keratin materials at least one colouring agent, preferably at least one pigment;
ii) applying to the keratin materials a composition comprising:
   a) at least one PHA as defined previously; and
   c) optionally at least one fatty substance which is preferably liquid at 25° C. at atmospheric pressure, preferably hydrocarbon-based; and/or
   e) optionally at least one additional solvent; and/or
   f) optionally at least one additive, preferably iv) at least one colouring agent, more preferentially at least one pigment; and
iii) applying to the keratin materials at least one crosslinking agent b); and
   e) optionally at least one additional solvent; and/or
   f) optionally at least one additive, preferably iv) at least one colouring agent, more preferentially at least one pigment;
      it being understood that steps i) to iii) are performed separately or simultaneously.

Steps i) to iii) may be performed in the order i) to iii) or in any order, preferably in the order i) to iii).

The process may also comprise one or more additional steps, preferably at least one waiting step and optionally a drying step.

More particularly, a subject of the invention is a non-therapeutic cosmetic process for treating keratin materials, in one or more successive steps, comprising the application to the keratin materials of a composition as defined previously. The treatment process is in particular a process for caring for or making up keratin materials.

### One-Action Application:

According to a particular embodiment of the invention, a) the PHA copolymer(s) bearing acetoacetate group(s) and derivative(s) as defined previously are applied to keratin materials, notably the skin or keratin fibres such as the hair. The PHA copolymer(s) bearing acetoacetate group(s) and derivative(s) as defined previously may be found in a preferably cosmetic composition as described previously, in which case said composition is applied to keratin materials.

According to a particular embodiment of the invention the applied composition comprises a) one or more PHA copolymer(s) bearing acetoacetate group(s) and derivative(s) as defined previously and b) one or more crosslinking agent(s) as defined previously. The ingredients a) and b) may be mixed together before application to keratin materials, notably the skin or keratin fibres such as the hair.

According to a particular embodiment of the invention, the applied composition comprises ingredients a) and b) as defined previously, and one or more fatty substances c) as defined previously, notably one or more oils. The ingredients a), b) and c) may be mixed together before application to keratin materials, notably the skin or keratin fibres such as the hair.

Preferably, the ingredients a) and b) are conveyed in the same solvent, preferably at least one volatile oil c). It has been found that the composition comprising a), b) and c) mixed just before application remains fluid long enough to be applied to keratin materials, notably the skin and keratin fibres such as the hair.

According to a particular embodiment of the invention, the composition comprising the ingredients a) and optionally b) and/or c) is applied to the keratin materials, and there is then a waiting period of between 10 seconds and 24 hours, particularly between 1 minute and 1 hour such as 30 minutes, at room temperature (25° C.). This step of evaporating ingredient c) or drying can be accelerated by subjecting the treated keratin material to heating after application, for example using a hair dryer or any other means suitable for heating treatments of keratin materials.

After evaporation of the solvents, notably c) the volatile oil(s) such as isododecane, the new material generated makes it possible to obtain “transfer-resistant”, non-tacky deposits that are resistant to edible oils and to water for skin application and resistant to shampoos and to water for hair application.

### Two-Action Application:

According to another particular embodiment of the invention, the process for treating keratin materials is performed in at least two successive steps:
in the first step (base coat):
   a) the PHA copolymer(s) according to the invention are applied to the keratin materials, notably the skin or keratin fibres such as the hair;
   preferentially, the PHA copolymer(s) according to the invention are found in a preferably cosmetic composition as described previously, in which case said composition is applied to the keratin materials; more preferentially, the composition applied during the first step comprises a) one or more PHA copolymer(s) according to the invention, and c) one or more fatty substance(s) as defined previously, which are preferably liquid, in particular isododecane, and optionally one or more colouring agents; and then
in a second step (top coat):
   b) the crosslinking agent(s) as defined previously are applied to the keratin materials, notably the skin or keratin fibres such as the hair.

According to a particular embodiment, the crosslinking agent(s) as defined previously are found in a composition, preferably a cosmetic composition as described previously, in which case said composition is applied to the keratin materials; more preferentially, the composition applied in the second step comprises b) one or more crosslinking agent(s) as defined previously, and optionally c) one or more fatty substance(s) as defined previously, which are preferably liquid, in particular isododecane.

According to a particular embodiment of the invention, between the first and second step, there is a waiting time of between 10 seconds and 24 hours, particularly between 1 minute and 1 hour such as 30 minutes, at room temperature (25° C.). This step of evaporating ingredient c) or drying can be accelerated by subjecting the treated keratin material to heating after application, for example using a hair dryer or any other means suitable for heating treatments of keratin materials. After the second step, the evaporation or drying step can be repeated.

The reaction of the crosslinking agent(s) b) on the A or B functions of the PHA copolymer(s) makes it possible to obtain, after evaporation or drying, transfer-resistant, non-tacky deposits that are resistant to food oils and to water for a skin application and resistant to shampoos and to water for a hair application.

### Three-Action Application:

According to a particular embodiment of the invention, the process for treating keratin materials involves at least three successive steps, comprising the following steps:

During the first step, a composition comprising one or more colouring agents is applied to the keratin materials; particularly, the composition applied during the first step comprises iv) one or more colouring agents chosen from pigments, direct dyes and mixtures thereof, preferably pigments, more preferentially chosen from carbon black, iron oxides, notably black or red iron oxide, and iron oxide-coated micas, triarylmethane pigments, notably blue and violet pigments such as Blue 1 Lake, azo pigments, notably red pigments such as D&C Red 7, the alkali metal salt of lithol red, such as the calcium salt of lithol red B, and even more preferentially red iron oxides.

Followed by a second (base coat) and a third step (top coat) corresponding to the first step and second step, respectively, of the previous two-step process.

Between the first and second step and/or between the second and third step, there may be a waiting time of between 10 seconds and 24 hours, particularly between 1 minute and 1 hour, such as 30 minutes, at room temperature (25° C.). These steps of evaporation of the ingredients c) or drying can be accelerated by subjecting the treated keratin material after application to heating by means of, for example, a hair dryer or any other means suitable for heating treatments of keratin materials. After the third step, the evaporation or drying step may be repeated.

The reaction of the crosslinking agent(s) b) with the functions A and/or B of the PHA copolymer(s) a) of the invention makes it possible to obtain, after evaporation of the volatile solvents or drying, transfer-resistant, non-tacky deposits that are resistant to food oils and to water for a skin application and resistant to shampoos and to water for a hair application.

In this system, it would be possible to trap non-volatile compounds to improve cosmeticity, provide sheen, volume, etc.

The non-volatile oils may preferentially be integrated into the composition of the base coat in the presence of the PHA polymer according to the invention during a two- or three-action application.

### EXAMPLES

The PHAs illustrated as reagents in the various examples were prepared in 3-litre chemostats and/or 5-litre Fernbach flasks depending on whether or not a μ-oxidation pathway inhibitor was used. The isolation of the PHAs is similar for all the examples obtained.

In a first step, the microorganism generates the PHAs which are stored in intracellular granules, the proportion of which varies as a function of the applied conditions such as the temperature or the nature of the culture medium. The generation of PHA granules may or may not be associated with the growth of the microorganism as a function of the nature of the microorganisms. During the second step, the biomass containing the PHAs is isolated, i.e. separated from the fermentation medium, and then dried. The PHAs are extracted from the biomass before being purified, if necessary.

A mixture of saturated and unsaturated carbon sources is, for certain examples, necessary for the stability of the PHA obtained.

TABLE 4

| Carbon source | CAS |
|---|---|
| Caprylic Acid (RADIACID 608) | 124-07-2 |
| Nonanoic acid | 112-05-0 |
| Undecylenic acid (10-Undecenoic acid) | 112-38-9 |

TABLE 5

| Carbon source | Genus and species | Source |
|---|---|---|
| Mixture of caprylic acid | Pseudomonas | ATCC ® 47054 ™ |
| and undecylenic acid | putida | |
| Mixture of nonanoic acid | Pseudomonas | ATCC ® 47054 ™ |
| and undecylenic acid | putida | |

Preparation of Reagent 1: PHA Bearing a Side Chain R¹ Representing a Linear 10% Unsaturated n-Octenyl Group and R² Representing an n-Pentyl Group

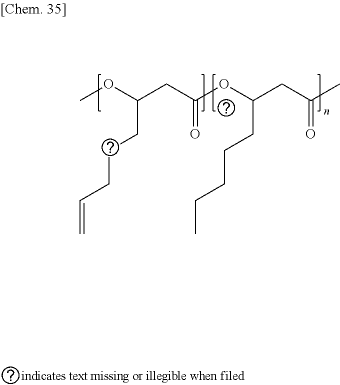

The process for synthesizing the compound of Example 1 is adapted from the article: Fed-batch production of unsaturated medium-chain-length polyhydroxyalkanoates with controlled composition by Pseudomonas putida KT2440, Z. Sun, J. A. Ramsay, M. Guay, B. A. Ramsay, Applied Microbiology Biotechnology, 82. 657-662, 2009.

The microorganism used is Pseudomonas putida KT2440 ATCC® 47054™ The culture method is performed under fed-batch growth axenic conditions with a maintenance solution containing a mixture of carbon source at a rate μ=0.15 h⁻¹ in a 3 L chemostat containing 2.5 L of culture medium.

The system is aerated with a flow of 0.5 vvm of air for a nominal dissolved oxygen (O_{D}) value at 30% of saturation. The pH is regulated with 15% aqueous ammonia solution. The temperature of the fermentation medium is regulated at 30° C.

### Assembly for the Fed-Batch Growth Fermentation Mode

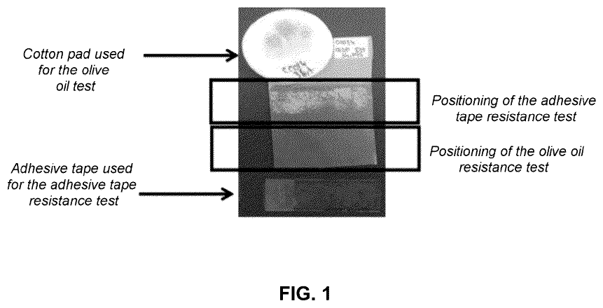

The fermentation medium is regulated in terms of temperature-pressure of dissolved oxygen and pH (not shown): see the attached FIG. 1.

The production process is performed using three different culture media. The first culture medium, defined CM1 “inoculum”, is used for the preparation of the preculture. The second culture medium, defined CM2 “batch”, is used for unfed batch growth of the microorganism with the primary carbon sources in the Fernbach flasks. The third culture medium, defined CM3 “maintenance”, is used for the fed-batch or maintenance fermentation mode with the carbon sources of interest at a flow rate calibrated as a function of the growth of the microorganism.

TABLE 6

| Ingredients in grams | CM1 | CM2 | CM3 |
|---|---|---|---|
| per litre | «inoculum» | «batch» | «maintenance» |
| (NH₄)₂SO₄ | 4.7 | 4.7 | |
| Na₂HPO₄; 7H₂O | 12 | 9 | |
| KH₂PO₄ | 2.7 | 2.03 | |
| MgSO₄; 7H₂O | 0.8 | 1.03 | |
| Nutrient Broth | 3 | / | |
| Caprylic acid | / | 0.9 | 900 |
| Undecylenic acid | / | 0.1 | 100 |
| Microelement solution | / | 10 | |
| Acrylic acid | / | / | |

| | |
|---|---|
| 2N NaOH | qs pH = 6.8 |
| MilliQ water | qs 1000 g |

The composition of the Nutrient Broth, as mass percentages, is 37.5% beef extract and 62.5% peptone. Reference 233000 DIFCO™.

TABLE 7

| Ingredients in grams per litre | Amount |
|---|---|
| FeSO₄•7H₂O | 10.0 g |
| CaCl₂•2H₂O | 3.0 g |
| ZnSO₄•7H₂O | 2.2 g |
| MnSO₄•4H₂O | 0.5 g |
| H₃BO₃ | 0.3 g |
| CoCl₂•6H₂O | 0.2 g |
| Na₂MoO₄•2H₂O | 0.15 g |
| NiCl₂•6H₂O | 0.02 g |
| CuSO₄•5H₂O | 1.00 g |
| MilliQ water (or 0.5N HCl) | qs 1000 g |

100 mL of preculture are prepared by suspending a cryotube containing 1 mL of the strain with 100 mL of “inoculum” culture medium at a pH adjusted to 6.8 with 2N NaOH in a 250 mL Fernbach flask and are then incubated at 30 C at 150 rpm for 24 hours. 1.9 L of CM2 “batch” culture medium placed in a presterilized 3 L chemostat are inoculated at O_{D}=0.1 with the 100 mL of preculture. After 4 hours at 30° C. at 850 rpm.

At the end of the introduction, the biomass is isolated by centrifugation and then washed three times with water. The biomass is dried by lyophilization before being extracted with ethyl acetate for 24 hours. The suspension is clarified by filtration on a GF/A filter (Whatman®). The filtrate, composed of PHA dissolved in the ethyl acetate, is concentrated by evaporation and then dried under high vacuum at 40° C. to constant mass.

The PHA may optionally be purified by successive dissolution and precipitation from an ethyl acetate/ethanol 70% methanol system, for example.

The PHA was fully characterized by spectroscopic and spectrometric methods and is in accordance with the expected chemical structure.

Preparation of Reagent 1′: PHA Copolymer Bearing a Side Chain R¹ Representing a 5% Unsaturated n-Octenyl Group and R² Representing an n-Hexyl Group

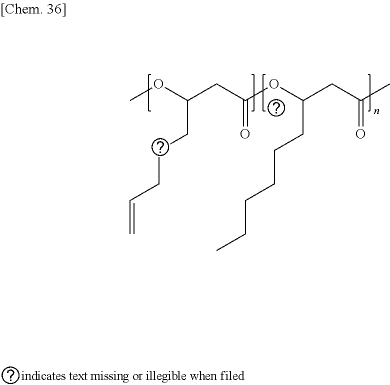

The copolymer of Example 1′ (5% unsaturation and R² chain representing n-hexyl) was prepared according to the procedure described for Example 1, with the same composition of the microelement solution as described in Example 1 and with the following culture medium compositions:

TABLE 6

| Ingredients | CM1 | CM2 | CM3 |
|---|---|---|---|
| in grams per litre | “inoculum” | “batch” | “maintenance” |
| (NH₄)₂SO₄ | 4.7 | 4.7 | / |
| Na₂HPO₄•7H₂O | 12 | 9 | / |
| KH₂PO₄ | 2.7 | 2.03 | / |
| MgSO₄•7H₂O | 0.8 | 1.03 | / |
| Nutrient Broth | 3 | / | / |
| Nonanoic acid | / | 0.95 | 950 |
| Undecylenic acid | / | 0.05 | 50 |
| Microelements Solution | / | 10 | / |

| | |
|---|---|
| 2N NaOH | qs pH = 6.8 |
| MilliQ water | qs 1000 g |

The PHA copolymer of Example 1′ was fully characterized by spectroscopic and spectrometric methods and is in accordance with the expected chemical structure, with a degree of unsaturation of 5%.

Reagent 1″: PHA Copolymer Bearing a Side Chain R¹ Representing a Linear 30% Unsaturated n-Octenyl Group and R² Representing an n-Pentyl Group

The copolymer of Example 1″ (30% unsaturated and R² chain representing n-pentyl) was prepared according to the procedure described for Example 1, with the same composition of the microelement solution as described in Example 1 and with the following culture medium compositions:

TABLE 8

| Ingredients | CM1 | CM2 | CM3 |
|---|---|---|---|
| in grams per litre | “inoculum” | “batch” | “maintenance” |
| (NH₄)₂SO₄ | 4.7 | 4.7 | / |
| Na₂HPO₄•7H₂O | 12 | 9 | / |
| KH₂PO₄ | 2.7 | 2.03 | / |
| MgSO₄•7H₂O | 0.8 | 1.03 | / |
| Nutrient Broth | 3 | / | / |
| Octanoic acid | / | 0.70 | 700 |
| Undecylenic acid | / | 0.3 | 300 |
| Microelements Solution | / | 10 | / |

| | |
|---|---|
| 2N NaOH | qs pH = 6.8 |
| MilliQ water | qs 1000 g |

The PHA copolymer was fully characterized by spectroscopic and spectrometric methods and is in accordance with the expected chemical structure.

Preparation of Reagent 2: PHA Bearing a Side Chain R¹ Representing a Linear 10% Unsaturated n-Octenyl Group and R² Representing an n-Hexyl Group

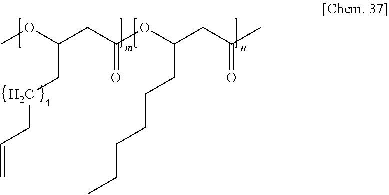

The process for producing reagent 1 is adapted to that for reagent 2, replacing the n-octanoic acid carbon source of reagent 1 with n-nonanoic acid to prepare reagent 2.

The PHA copolymer of reagent 2 was fully characterized by spectroscopic and spectrometric methods and is in accordance with the expected chemical structure, with a degree of unsaturation of 5%.

Preparation of Reagent 2′: PHA Bearing a Side Chain R¹ Representing a Linear 2% Unsaturated n-Octenyl Group and R² Representing an n-Hexyl Group

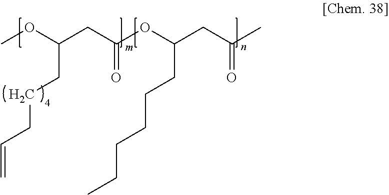

The copolymer of Example 2′ (2% unsaturation and R² chain representing n-hexyl) was prepared according to the procedure described for Example 1, with the same composition of the microelement solution as described in Example 1 and with the following culture medium compositions:

TABLE 9

| | MC1 | MC2 | MC3 |
|---|---|---|---|
| | «inoculum» | «batch» | «maintenance» |
| (NH₄)₂SO₄ | 4.7 | 4.7 | |
| Na₂HPO₄; 7H₂O | 12 | 9 | |
| KH₂PO₄ | 2.7 | 2.03 | |
| MgSO₄; 7H₂O | 0.8 | 1.03 | |
| Nutrient Broth | 3 | / | |
| Acide nonanoïque | / | 0.98 | 980 |
| Acide undécylènique | | 0.02 | 20 |
| Solution Microéléments | / | 10 | |
| Acide acrylique | / | / | |

| | |
|---|---|
| NaOH 2N | QSP pH = 6.8 |
| Eau milliQ | QSP m = 1000 g |

The PHA copolymer of Example 2′ was fully characterized by spectroscopic and spectrometric methods and is in accordance with the expected chemical structure, with a degree of unsaturation of 2%.

Preparation of Reagent 3: Copolymer of PHA Bearing a Side Chain R¹ Representing an Isohexenyl Group and R² Representing an Isobutyl Group

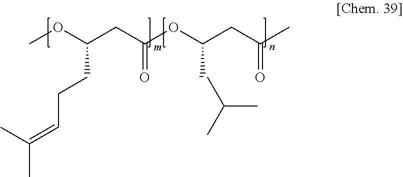

The production process of Example 3 is an adaptation of Applied and Environmental Microbiology, Vol. 60, No. 9. 3245-3254 (1994) “Polyester Biosynthesis Characteristics of Pseudomonas citronellolis Grown on Various Carbon Sources, Including 3-Methyl-Branched Substrate”. Mun Hwan Choi and Sung Chul Yoon. The microorganism used is Pseudomonas citronellolis ATCC® 13674™. The culture method is performed under unfed-batch axenic culture conditions in 5 L Fernbach flasks (Corning® ref. 431685) containing 2 L of culture medium, shaken at 110 rpm at 30° C. in an orbital incubator (orbit diameter of 2.5 cm).

The production process is performed using two different culture media. The first culture medium, defined CM1 “inoculum”, is used for the preparation of the preculture. The second culture medium, defined CM2 “batch”, is used for unfed batch culture growth of the microorganism with the carbon source of interest in the Fernbach flasks.

TABLE 10

| Ingredients in grams | | |
|---|---|---|
| per litre | CM1 «inoculum» | CM2 «batch» |
| (NH₄)₂SO₄ | / | 0.66 |
| Na₂HPO₄•7H₂O | / | 7.3 |
| KH [symbol] PO4 | / | 2.3 |
| NaHCO₃ | / | 0.3 |
| CaCl₂•2H₂O | / | 0.1 |
| MgSO₄•7H₂O | / | 0.25 |
| Citric acid | / | 1.03 |
| Citronellol | / | 5.5 |
| Microelements solution | / | 1 |
| Nutrient broth | 1.5 | / |
| Yeast extract | 1 | / |

| | |
|---|---|
| 2N NaOH | qs pH = 6.8 |
| MilliQ water | qs m = 1000 g |

[symbol]

indicates data missing or illegible when filed

The composition of the Nutrient Broth, as mass percentages, is 37.5% beef extract and 62.5% peptone. Reference 233000 DIFCO™ BD.

The composition of the yeast extract, as a mass percentage, is 100% autolysate of the yeast Saccharomyces cerevisiae. Reference 210933 DIFCO™ BD.

TABLE 11

| Ingredients in grams | |
|---|---|
| per litre | Amount |
| FeSO₄•7H₂O | 5.56 g |
| CaCl₂•2H₂O | 3.0 g |
| ZnSO₄•7H₂O | 0.58 g |
| MnCl₂•4H₂O | 3.86 g |
| H₃BO₃ | 0.6 g |
| CoCl₂•6H₂O | 5.62 g |
| Na₂MoO₄•2H₂O | 0.06 g |
| NiCl [symbol] •6H₂O | 0.04 g |
| CuSO₄•5H₂O | 0.34 g |
| 0.5N HCl | qs 1000 g |

[symbol]

indicates data missing or illegible when filed

100 mL of preculture are prepared by suspending a cryotube containing 1 mL of the strain with 100 mL of “inoculum” culture medium at a pH adjusted to 6.8 with 2N NaOH in a 250 mL Fernbach flask and then incubated at 30° C. at 150 rpm for 24 hours. 1.9 L of CM2 “batch” culture medium placed in a presterilized 5 L Fernbach flask are inoculated at O_{D}=0.1 with 100 mL of inoculum.

After 70 hours at 30° C. at 110 rpm, the biomass is dried by lyophilization before being extracted with dichloromethane for 24 hours. The suspension is clarified by filtration on a GF/A filter (Whatman®). The filtrate, composed of PHA dissolved in dichloromethane, is concentrated by evaporation and then dried under high vacuum at 40° C. to constant mass.

The PHA may optionally be purified by successive dissolution and precipitation, for instance using a dichloromethane/methanol system.

Reagent 3 was fully characterized by spectroscopic and spectrometric methods and is in accordance with the expected chemical structure, with 68 mol % of unit (A) for which R¹=isohexenyl and 32 mol % of unit (B) for which R²=isobutyl.

### Preparation of Reagent 4: Poly(3-Hydroxynonanoate-Co-Undecenoate) Containing 5% Unsaturations 100% Epoxidized

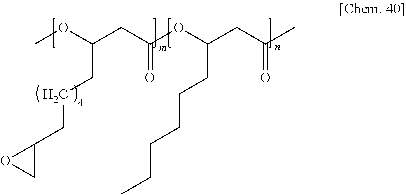

20 g of the PHA copolymer from the Reagent 1′ preparation were dissolved in 80 mL of anhydrous dichloromethane. A suspension of 1.9 g of 77% m-CPBA was prepared with 20 mL of anhydrous dichloromethane and added to the mixture with stirring, at room temperature for at least 120 hours.

The reaction medium was then precipitated from a 500 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

Reagent 4 was fully characterized by spectroscopic and spectrometric methods and is in accordance with the expected chemical structure. Epoxidation to 100%.

### Preparation of Reagent 5: Poly(3-Hydroxynonanoate-Co-Undecenoate) Containing 10% Unsaturations 100% Epoxidized

10 g of the PHA copolymer prepared according to the reagent 2 preparation were dissolved in 40 mL of anhydrous dichloromethane. A suspension of 1.9 g of 77% m-CPBA was prepared with 10 mL of anhydrous dichloromethane and added to the mixture with stirring, at room temperature for at least 120 hours.

The reaction medium was then precipitated from a 500 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

Reagent 5 was fully characterized by spectroscopic and spectrometric methods and is in accordance with the expected chemical structure. Epoxidation to 100%.

### Preparation of Reagent 6: Poly(3-Hydroxyoctanoate-Co-Undecenoate) Containing 30% Unsaturations 100% Epoxidized

10 g of the PHA copolymer identical to that of Example 1″ but with a degree of unsaturation of 30% were dissolved in 40 mL of anhydrous dichloromethane. A suspension of 6.2 g of 77% m-CPBA was prepared with 10 mL of anhydrous dichloromethane and added to the mixture with stirring, at room temperature for at least 120 hours.

The reaction medium was then precipitated from a 250 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

Reagent 6 was fully characterized by spectroscopic and spectrometric methods and is in accordance with the expected chemical structure. Epoxidation to 100%.

Preparation of Reagent 7: PHA Bearing a Side Chain R¹ Representing a Linear 5% Unsaturated 8-Bromo-n-Octanoyl Group and R² Representing an n-Hexyl Group

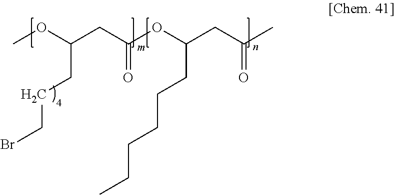

The process for synthesizing the compound of Example 1 is adapted from the article: Fed-batch production of unsaturated medium-chain-length polyhydroxyalkanoates with controlled composition by Pseudomonas putida KT2440, Z. Sun, J. A. Ramsay, M. Guay, B. A. Ramsay, Applied Microbiology Biotechnology, 82. 657-662, 2009.

The microorganism used is Pseudomonas putida KT2440 ATCC®47054™. The culture method is performed under fed-batch growth axenic conditions with a maintenance solution containing a mixture of carbon source at a rate μ=0.15 h¹ in a 3 L chemostat containing 2.5 L of culture medium.

The system is aerated with a flow of 0.5 vvm of air for a nominal dissolved oxygen (O_{D}) value at 30% of saturation. The pH is regulated with a solution composed of ammonia and glucose at 15% and 40% final mass, respectively. The temperature of the fermentation medium is regulated at 30° C.

### Equipment for the Fed-Batch Growth Fermentation Mode:

The fermentation medium is regulated in terms of temperature-pressure of dissolved oxygen and pH (not shown).

The production process is performed using three different culture media. The first culture medium, defined CM1 “inoculum”, is used for the preparation of the preculture. The second culture medium, defined CM2 “batch”, is used for unfed batch growth of the microorganism with the primary carbon sources in the Fernbach flasks. The third culture medium, defined CM3 “maintenance”, is used for the fed-batch or maintenance fermentation mode with the carbon sources of interest at a flow rate calibrated as a function of the growth of the microorganism.

TABLE 12

| Ingredients in grams | CM1 | CM2 | CM3 |
|---|---|---|---|
| per litre | «inoculum» | «batch» | «maintenance» |
| (NH₄)₂SO₄ | 4.7 | 4.7 | |
| Na [symbol] HPO₄•7H₂O | 12 | 9 | |
| KH [symbol] PO₄ | 2.7 | 2.03 | |
| MgSO₄•7H₂O | 0.8 | 1.03 | |
| Nutrient Broth | 3 | / | |
| Nonanoic acid | / | 1 | 923 |
| 11-Bromoundecanoic acid | / | 0 | 77 |
| Microelements solution | / | 10 | |

| | |
|---|---|
| 2N NaOH | qs pH = 6.8 |
| MilliQ water | qs 1000 g |

[symbol]

indicates data missing or illegible when filed

The composition of the Nutrient Broth, as mass percentages, is 37.5% beef extract and 62.5% peptone. Reference 233000 DIFCO™.

TABLE 13

| Ingredients in grams | |
|---|---|
| per litre | Amount |
| FeSO₄•7H₂O | 10.0 g |
| CaCl₂•2H₂O | 3.0 g |
| ZnSO₄•7H₂O | 2.2 g |
| MnSO₄•4H₂O | 0.5 g |
| H₃BO₃ | 0.3 g |
| CoCl₂•6H₂O | 0.2 g |
| Na₂MoO₄•2H₂O | 0.15 g |
| NiCl₂•6H₂O | 0.02 g |
| CuSO₄•5H₂O | 1.00 g |
| MilliQ water (or 0.5N HCl) | qs 1000 g |

100 mL of preculture are prepared by suspending a cryotube containing 1 mL of the strain with 100 mL of “inoculum” culture medium at a pH adjusted to 6.8 with 2N NaOH in a 250 mL Fernbach flask and are then incubated at 30 C at 150 rpm for 24 hours. 1.9 L of CM2 “batch” culture medium placed in a presterilized 3 L chemostat are inoculated at Op=0.1 with the 100 mL of preculture. After 4 hours at 30 C at 850 rpm, introduction of the maintenance culture medium is performed, applying the flow rate defined by equation 1.

At the end of the introduction, the biomass is isolated by centrifugation and then washed three times with water. The biomass is dried by lyophilization before being extracted with ethyl acetate for 24 hours. The suspension is clarified by filtration on a GF/A filter (Whatman®). The filtrate, composed of PHA dissolved in the ethyl acetate, is concentrated by evaporation and then dried under high vacuum at 40° C. to constant mass.

The PHA may optionally be purified by successive dissolution and precipitation from an ethyl acetate/ethanol 70% methanol system, for example.

The PHA was fully characterized by spectroscopic and spectrometric methods and is in accordance with the expected chemical structure: 95 mol % of unit (B) for which R²=n-hexyl (71%) and n-butyl (24%) and 5 mol % of unit (A) for which R¹=8-bromo-n-octanyl (5.9%) and 6-bromo-n-hexyl (0.2%).

### Example 1: Poly(3-hydroxynonanoate-co-undecenoate) Containing 10% Unsaturations 100% Grafted with 3-mercaptopropyloxobutanoate (MPOB-Grafted PHNUn10)

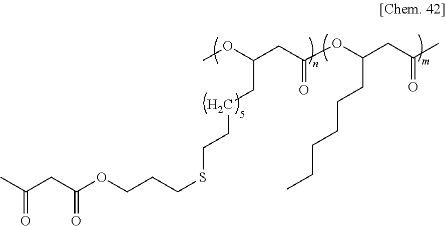

Preparation of Reagent 7: 3-mercaptopropyloxobutanoate (MPOB)

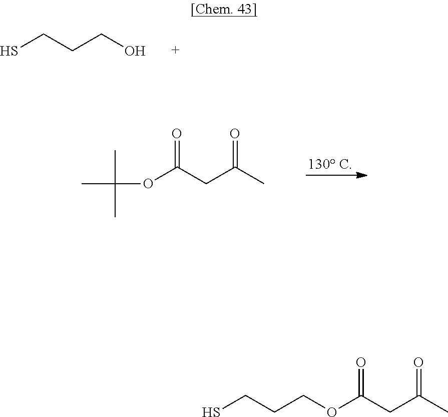

5 g of 3-mercaptopropanol and 10 g of tert-butyl acetoacetate were dissolved at room temperature with stirring. The reaction medium was then heated at 130° C. for at least 4 hours. The excess tert-butyl acetoacetate and the tert-butanol (formed in-situ) were removed under reduced pressure to give MPOB: reagent 7.

Functionalization of Mcl-PHA Bearing a Linear C₆ Side Chain R^{2′} and 10% Unsaturated C₈ Chain R¹′(PHNUn10) Grafted with 3-mercaptopropyloxobutanoate (MPOB-Grafted PHNUn10)

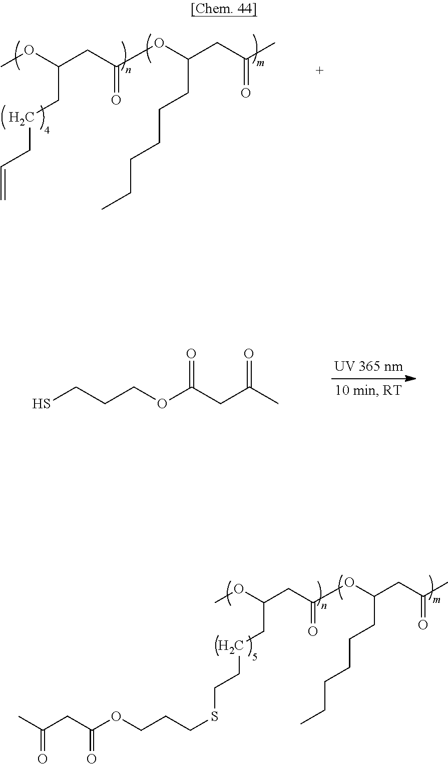

1 g of reagent 2 and 0.5 g of reagent 7 were dissolved in 10 mL of ethyl acetate at room temperature with stirring. 25 mg of 2-hydroxy-2-methylpropiophenone were added to the mixture. The medium was then irradiated under a 100 W UV lamp at 365 nm and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The PHA of Example 1 was characterized by spectrometric and spectroscopic methods. The characteristic spectrometric signals of the unsaturations totally disappeared. Grafting to 100%.

### Example 1′: Poly(3-HydroxyNonanoate-co-Undécenoate) à 10% D'Insaturations Greffés á 100% avec le 2,3-mercaptopropyl(dioxobutanoate)

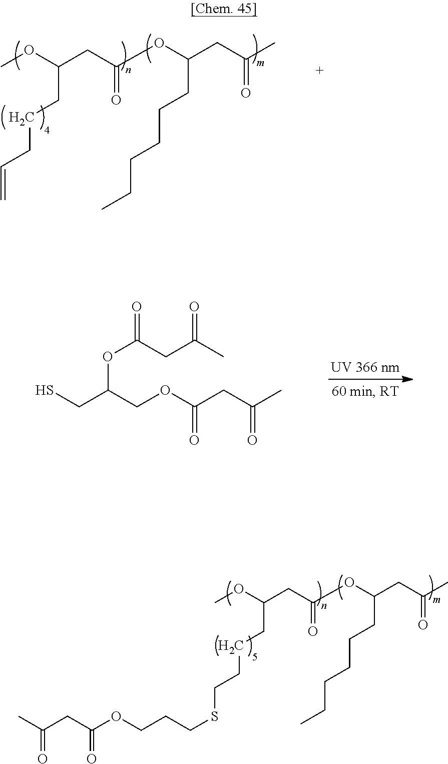

Préparation du Réactif 7′: 2,3-mercaptopropyl(dioxobutanoate)

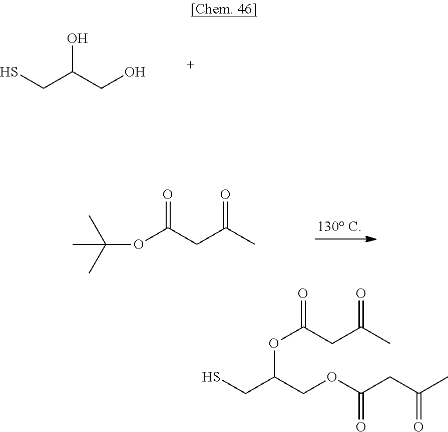 [SMILES: OCC(O)CS ; CC(=O)CC(=O)OC(C)(C)C ; CC(=O)CC(=O)OCC(CS)OC(=O)CC(C)=O]

15 g of thioglycerol and 50 g of tert-butyl acetoacetate were dissolved at room temperature with stirring. The reaction medium was then heated at 130° C. for at least 4 hours. The excess tert-butyl acetoacetate and the thioglycerol (formed in-situ) were removed under reduced pressure to give reagent 7′.
Functionalization of Mcl-PH a Bearing a Linear C₆ Side Chain R^{2′} and 10% Unsaturated C₈ Chain R¹′(PHNUn10) Grafted with 2,3-Mercaptopropyl(Dioxobutanoate) (MPOB-Grafted PHNUn10)

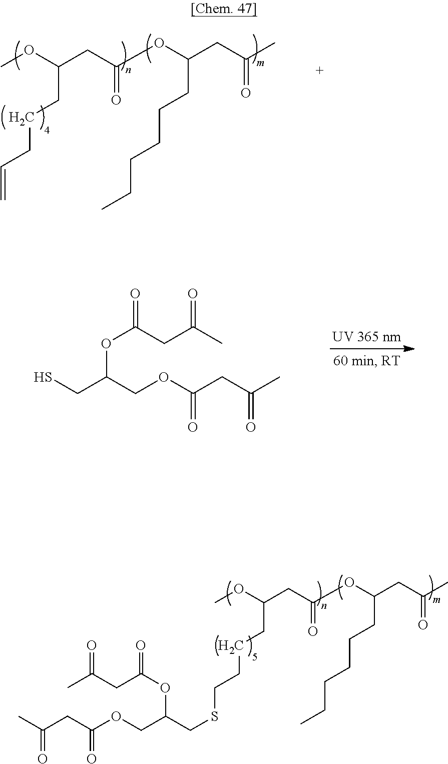

2 g of reagent 2′ and 1 g of reagent 7′ were solubilized in 10 ml of ethyl acetate at room temperature with stirring. 25 mg of 2-Hydroxy-2-methylpropiophenone was added to the mixture. The medium was then irradiated under a 100 W UV lamp at 365 nm with stirring for at least 60 minutes.

The reaction medium was then dried under dynamic vacuum at 40° C. to obtain a homogeneous film.

The PHA of Example 1′ was characterized by spectroscopic and spectrometric methods. The spectrometric characteristic signals of the unsaturations completely disappeared. 100% grafting.

### Example 2 (Comparative) with Poly(3-HydroxyNonanoate-Co-Undécenoate) Containing 2% of Unsaturations (Reagent 2′)

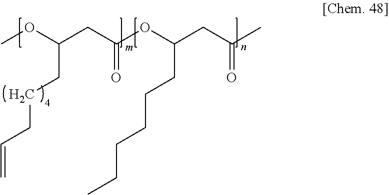

### Performance Evaluations

Crosslinking agents b):

TABLE 14

| | |
|---|---|
| Polythiol crosslinking agent [4-6% (MERCAPTOPROPYL)METHYLSILOXANE]- DIMETHYLSILOXANE COPOLYMER (CAS Number: 102783-03-9) PRODUCT CODE: SMS- 042 Supplier: Gelest | 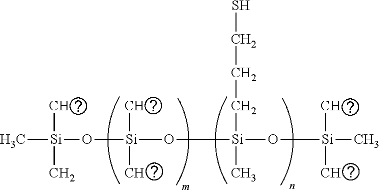 |
| Amino crosslinking agent: BIS-CETEARYL AMODIMETHICONE Supplier: MOMB MATERIALS | 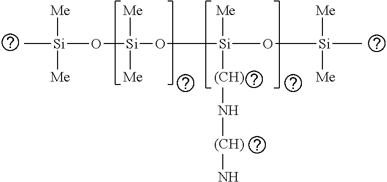 |
| Trimethylolpropane triacrylate Supplier: Sigma-Aldrich | 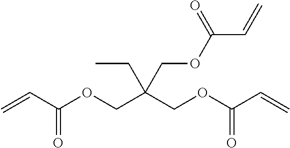 [SMILES: C=CC(=O)OCC(CC)(COC(=O)C=C)COC(=O)C=C] |

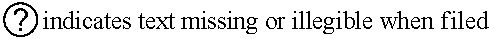

### Example 2: Hair Application

### Hairstyling Evaluation Protocol

A 1 g lock of keratin fibres (90% Natural White NW hair) was wrapped around a brush (approximate diameter of 2 cm over a length of 3 cm).

A comparative composition 2 was prepared with isododecane and ethanol (90/10 by mass).

2 g of a composition A comprising the PHA copolymer of Example 1 at 10% by weight in a mixture of isododecane/ethanol (90/10). Composition A was then sprayed onto a keratin fibre lock.

2 g of another composition B comprising the PHA copolymer of Example 1 at 10% by weight in a mixture of isododecane/ethanol (90/10) and 5% of crosslinking agent. (polythiol). Composition B was then sprayed onto a keratin fibre lock.

2 g of a composition A′ comprising the PHA copolymer of Example 1′ at 10% by weight in a mixture of isododecane/ethanol (90/10). Composition A′ was then sprayed onto a keratin fibre lock.

2 g of another composition B′ comprising the PHA copolymer of Example 1′ at 10% by weight in a mixture of isododecane/ethanol (90/10) and 5% of crosslinking agent. (polyacrtlate+DBU). Composition B′ was then sprayed onto a keratin fibre lock.

2 g of a composition A″ comprising the PHA copolymer of Example 2 at 10% by weight in a mixture of isododecane/ethanol (90/10). Composition A″ was then sprayed onto a keratin fibre lock.

The locks were weighed before and after application (0.5 g of comparative composition 2, A and B were applied to the lock).

The lockwas then measured after application and 24 hours after application (lock left at room temperature, 25° C.).

A comparison was made with a lock onto which only a mixture of isododecane and ethanol (90/10) was sprayed.

TABLE 15

| | Value after | Value after 24 |
|---|---|---|
| | application | hours |
| Examples | (in cm) | (in cm) |
| Untreated lock | 20.5 | 19 |
| Comparative 1 | | |
| Lock treated with c) isododecane and | 20.5 | 16 |
| alkanol: ethanol | | |
| Comparative 2 | | |
| Lock treated with composition | 20.5 | 10 |
| A″comprising the PHA copolymer of | | |
| Example 2 | | |
| Comparative 3 | | |
| Lock treated with composition A | 20.5 | 7 |
| comprising the PHA copolymer of | | |
| Example 1 | | |
| Invention | | |
| Lock treated with composition A′ | 20.5 | 8 |
| containing the PHA of Example 1′. | | |
| Invention | | |
| Lock treated with composition B′ | 20.5 | 3 |
| containing the PHA of Example 1′ and | | |
| a prolyacrylate crosslinking agent (3) | | |
| Invention | | |

The lower the value, the more the shape is maintained over time. It is seen that the locks treated with the PHA copolymer a) according to the invention significantly improve the shape of the curls, which appear sharper and closer together, unlike the comparative locks 1, 2 and 3. In addition, even after 24 hours, the lock lost significantly less curl shape than the comparative locks. In addition, the addition of crosslinker allows to maintain the shape in time in a significant way.

### Example 3: Skin Application

Evaluations in simplex formulations were conducted to show the performance for application to the skin.

### For One-Action Applications

A formula containing a polymer bearing acetoacetate functions according to the invention, a pigment, a solvent, an oil and a crosslinking agent and optionally a catalyst was produced.

This formula was applied to a Bioskin type vitro support (elastomeric skin-equivalent support) using a film drawer (wet thickness 100 μm). The deposit was left to dry for 24 hours.

The simplex formulation is prepared using a Speed Mixer (2 minutes-3500 rpm).

### For Two-Action Applications

In a first stage, a base coat formulation containing a polymer bearing acetoacetate functions according to the invention was produced. This formulation was applied to an in vitro support of the Bioskin type (elastomeric skin-equivalent support) using a film drawer (wet thickness 100 μm). The deposit was left to dry for 24 hours.

After 24 hours of drying, the top coat containing a crosslinking agent was applied.

After drying for 24 hours, evaluations of the deposits are performed:

### Resistance to Olive Oil/Water

0.5 mL of olive oil or water is applied to the film of formulation. After 5 minutes, the olive oil or water is removed by wiping 15 times with cotton wool. The deterioration of the film following contact with the olive oil or the water is thus examined.

### Resistance to Adhesive Tape

A piece of adhesive tape (Scotch® Magic™ 810 from 3M; w=19 mm, L=5 cm) was applied to the deposit. A weight of approximately 1070 g was placed on the piece of adhesive tape for 30 seconds. The adhesive tape was then removed and mounted on a slide holder so as to properly observe the result. The adherence of the film to the support is thus evaluated: see FIG. 1.

### Fragmentation Test of the Formulations on BioSkin

As for the olive oil and adhesive tape resistance tests, films are made on a Bioskin sample. After a day of drying, the Bioskin sheet is stretched 10 times by hand force.

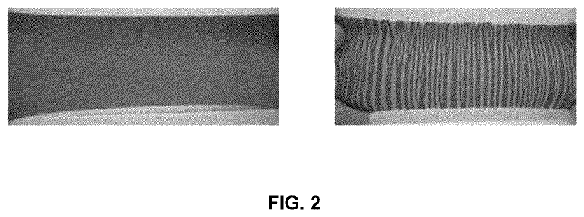

The result may then be observed on the formulation film (fragmentation or otherwise). Two response examples are presented in FIG. 2.

Results of the fragmentation test of FIG. 2; On the left: cohesive deposit, no fragmentation/On the right: fragmentation with appearance of deposit breakage under stress

For each combination, the evaluations were conducted for the base coat alone after drying and on the [Base coat]+[Top coat] system after drying, to evaluate the improvement of the cosmetic properties by such combinations. In all cases, the improvement of at least one property was observed by adding the top coat.

The evaluation was made in the following manner:
+++: cosmetic property evaluated as very effective
++: cosmetic property evaluated as moderately effective
+: cosmetic property evaluated as not very effective
0: cosmetic property evaluated as ineffective

### Formulations Containing Only Volatile Oil:

### Example 3-1: Two-Action Application of Polymer of Example 1 or Comparative Example 2+Amino Compound 2/Red Iron Oxide Pigment

### Base Coat

TABLE 16

| | by weight per 100 g of |
|---|---|
| Ingredients | composition |
| PHA of Example 1 | 20 |
| Red iron oxide (CI: 77491) | 6 |
| Ethanol | 7.4 |
| Isododecane | qs 100 |

### Comparative 1 Comprising a PHA without ACAC Function

TABLE 17

| Ingredients | % ma* |
|---|---|
| PHA of Example 2 (Reagent 2′) | 20 |
| Red iron oxide (CI: 77491) | 6 |
| Ethanol | 7.5 |
| Isododécane | Qsp 100 |

*% en poids de matière active

### Top Coat

TABLE 18

| | by weight per 100 g of |
|---|---|
| Ingredients | composition |
| Amine crosslinking agent (2) | 5 |
| Isododecane | qs 100 |

The evaluation results are summarized in the tables below:

TABLE 19

| Property | Fragmentation. | Water resistance |
|---|---|---|
| Base coat: | +++ | +++ |
| PHA of Ex. | | |
| 1 | | |

It is seen that the PHAs a) of the invention make it possible notably to obtain very effective cosmetic properties in terms of fragmentation and water resistance.

TABLE 20

| | Adhesive | | Water | Oil | Sebum |
|---|---|---|---|---|---|
| Property | tape test | Fragment. | resistance | resistance | resistance |
| Base coat + | +++ | +++ | +++ | +++ | +++ |
| top coat: | | | | | |
| polyamine(2) | | | | | |

If a crosslinking agent is added, notably a amined crosslinking agent such as (2), it is seen that not only are the cosmetic properties in terms of fragmentation and water resistance very good, but also those of the test of adhesion to the support (adhesive tape test), the oil resistance and the sebum resistance. Deposits that are resistant to daily chemical (water/olive oil/sebum) and mechanical (fragmentation/adhesive tape test) attacking factors are thus obtained.

Moreover, it appeared that the oil resistance and sebum resistance are more effective in comparison with the Comparative 1 comprising the PHA of example 2 without acac function.

### Example 3-2: One-Action Application of PHA of Example 1+Thiolated Crosslinking Agent/Pigment: Red Iron Oxide

TABLE 21

| Ingredients | % AM* |
|---|---|
| PHA of Example 1 | 20 |
| Thiolated crosslinking agent (1) | 5 |
| Ethanol | 7.5 |
| Isododecane | qs 100 |

*weight % of active material

The evaluation results are summarized in the table below:

TABLE 22

| Property | Fragment. | Water resistance |
|---|---|---|
| Base coat: | +++ | +++ |
| PHA of Ex. | | |
| 1 | | |

It is seen that the PHAs a) of the invention make it possible notably to obtain very effective cosmetic properties in terms of fragmentation and water resistance.

TABLE 23

| | Adhesive | | Water | Oil | Sebum |
|---|---|---|---|---|---|
| Property | tape test | Fragment. | resistance | resistance | resistance |
| Base coat + | +++ | +++ | +++ | +++ | ++ |
| top coat: | | | | | |
| Polythiol | | | | | |
| (1) | | | | | |

Comparative 2 with a PHA without AcAc Function+Thiol Crosslinker

### Application 1 PHA Gesture of Example 2+Thiol Cross-Linking Agent/Pigment: Red Iron Oxide

TABLE 24

| Ingredients | % ma* |
|---|---|
| PHA of Example 2 | 20 |
| Red iron oxide (CI: 77491) | 6 |
| Thiolated crosslinking agent (1) | 5 |
| Ethanol | 7.5 |
| Isododecane | Qsp 100 |

*weight % of active material

If a crosslinking agent is added, notably a thiolated crosslinking agent such as (1), it is seen that not only are the cosmetic properties in terms of fragmentation and water resistance very good, but also those of the test of adhesion to the support (adhesive tape test) and the oil resistance test, and the formulation is moderately effective against sebum.

Moreover, it appeared that the oil resistance are more effective in comparison with the Comparative 2 comprising the PHA of example 2 without acac function. Deposits that are resistant to daily chemical (water/olive oil/sebum) and mechanical (fragmentation/adhesive tape test) attacking factors are thus obtained.

### Example 3-3: Two-Action Application of the Polymer of Example 1+Amine-Based Crosslinking Agent

TABLE 25
Base Coat

| | by weight per 100 g of |
|---|---|
| Ingredients | composition |
| PHA of Example 1 | 20 |
| Ethanol | 8 |
| Isododecane | qs 100 |

### Top Coat

TABLE 26

| | by weight per 100 g of |
|---|---|
| Ingredients | composition |
| Amine crosslinking agent (2) | 5 |
| Isododecane | qs 100 |

The evaluation results are summarized in the tables below:

It is seen that the PHAs a) of the invention make it possible notably to obtain very effective cosmetic properties in terms of fragmentation and water resistance.

TABLE 27

| Property | Water resistance | Oil resistance | Sebum resistance |
|---|---|---|---|
| Base coat PHA of | +++ | +++ | +++ |
| Example 1 + top | | | |
| coat: | | | |
| Polyamine (2) | | | |

If a crosslinking agent is added, notably an amine-based crosslinking agent such as (2), it is seen that not only are the cosmetic properties in terms of fragmentation and water resistance very good, but also those in terms of the oil resistance and the sebum resistance. Deposits that are resistant to daily chemical attacking factors (water/olive oil/sebum) are thus obtained.

1. A polyhydroxyalkanoate (PHA) copolymer a) which contains several repeating units below, and also the optical or geometrical isomers thereof, the organic or mineral acid or base salts thereof, and the solvates thereof:
—[—O—CH(R¹)—CH₂—C(O)—]— unit (A)
in which polymer units (A):
R¹ represents a saturated or unsaturated, linear or branched, non-cyclic hydrocarbon-based chain, or a saturated or unsaturated non-aromatic cyclic hydrocarbon-based chain, comprising from 3 to 30 carbon atoms;
said hydrocarbon-based chain being:
substituted with one or more groups chosen from:
A) R^{3′}—C(X)—C(R⁴)(R⁵)—C(X′)—[Y]ₙ—* and B) R^{3′}—C(X)—C(—[Y]ₙ—*)(R⁴)—C(X)—R⁶ with:
R^{3′} and R⁶, which may be identical or different, representing a group chosen from optionally substituted (C₁-C₆)alkyl, optionally substituted (C₁-C₆)alkoxy, optionally substituted (C₁-C₆)alkylthio, optionally substituted (di)(C₁-C₆)(alkyl)amino, (hetero)aryl, (hetero)cycloalkyl, (hetero)aryloxy, (hetero)cycloalkyloxy, (hetero)arylthio, (hetero)cycloalkylthio, (hetero)arylamino, (hetero)cycloalkylamino;
R⁴ and R⁵, which may be identical or different, represent a hydrogen atom or a group chosen from (C₁-C₆)alkyl;
n is 0 or 1;
X and X′, which may be identical or different, represent an oxygen or sulfur atom or a group N—Rₐ with Rₐ representing a hydrogen atom or a (C₁-C₄)alkyl group;
Y represents a heteroatom chosen from O, S, N—Rₐ with Rₐ as defined previously;
* represents the point of attachment of the group A) or B) connected to the rest of the PHA copolymer(s); and
the radicals R¹ also possibly being:
substituted with one or more atoms or groups chosen from: a) halogen, b) hydroxyl, c) thiol, d) (di)(C₁-C₄)(alkyl)amino, e) (thio)carboxy, f) (thio)carboxamide —C(O)—N(Rₐ)₂ or C(S)—N(Rₐ)₂, g) cyano, h) iso(thio)cyanate, i) (hetero)aryl, and j) (hetero)cycloalkyl, k) cosmetic active agent; 1) R—X with R representing a group chosen from α) cycloalkyl, β) heterocycloalkyl, γ) (hetero)aryl, and m) thiosulfate; X representing a′) O, S, N(R′ₐ) or Si(R′_{b})(R′_{c}), b′) S(O)ᵣ, or (thio)carbonyl, c′) or combinations of a′) with b′); R′ₐ representing a hydrogen atom, or a (C₁-C₄)alkyl group or an aryl(C₁-C₄)alkyl group; R′_{b} and R′_{c}, which may be identical or different, represent a (C₁-C₄)alkyl or (C₁-C₄)alkoxy group; and/or
optionally interrupted with one or more a′) heteroatoms, b′) S(O)ᵣ, (thio)carbonyl, c′) or combinations of a′) with b′) with r being equal to 1 or 2, Rₐ being as defined previously, R_{b} and R_{c} being as defined previously;

2. The polyhydroxyalkanoate PHA copolymer a) according to claim 1, which contains, at least two different repeating polymer units chosen from the units (A) and (B) below, and also the optical or geometrical isomers thereof, the organic or mineral acid or base salts thereof, and the solvates thereof:
—[—O—CH(R¹)—CH₂—C(O)—]— unit (A)
—[—O—CH(R²)—CH₂—C(O)—]— unit (B)
in which polymer units (A) and (B):
it being understood that (A) is different from (B).

3. The polyhydroxyalkanoate PHA copolymer a) as defined in claim 1, which contains the repeating unit of formula (I), and also the optical or geometrical isomers thereof, the organic or mineral acid or base salts thereof, and the solvates thereof: 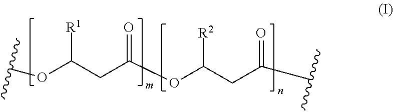
in which formula (I):
m and n are integers greater than or equal to 1.

4. The polyhydroxyalkanoate PHA copolymer according to claim 1, which contains three different repeating polymer units (A), (B) and (C), and also the optical or geometrical isomers thereof and the solvates thereof:
—[—O—CH(R¹)—CH₂—C(O)—]— unit (A)
—[—O—CH(R²)—CH₂—C(O)—]— unit (B)
—[—O—CH(R³)—CH₂—C(O)—]— unit (C)
in which polymer units (A), (B) and (C):
R³ represents a saturated or unsaturated, cyclic or non-cyclic, linear or branched hydrocarbon-based chain comprising from 1 to 30 carbon atoms; said chain being optionally substituted with one or more groups chosen from A) or B) as defined previously, optionally substituted with the groups a) to j) as defined for R¹ and/or optionally interrupted with one or more heteroatoms or groups a′) to c′) as defined for R¹.

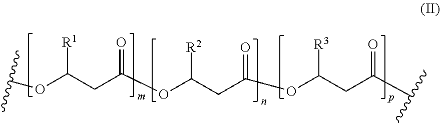

5. The polyhydroxyalkanoate PHA copolymer a) according to claim 1, which contains four different repeating polymer units (A), (B), (C) and (D), and also the optical or geometrical isomers thereof, the organic or mineral acid or base salts thereof, and also the solvates thereof:
—[—O—CH(R¹)—CH₂—C(O)—]— unit (A)
—[—O—CH(R²)—CH₂—C(O)—]— unit (B)
—[—O—CH(R³)—CH₂—C(O)—]— unit (C)
—[—O—CH(R⁴)—CH₂—C(O)—]— unit (D)
in which polymer units (A), (B), (C) and (D):
R⁴ is as defined for R², and
it being understood that:
(A) is different from (B), (C) and (D), (B) is different from (A), (C) and (D), (C) is different from (A), (B) and (D), and (D) is different from (A), (B) and (C).

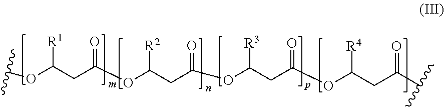

6. The polyhydroxyalkanoate PHA copolymer a) according to claim 1, which contains five different repeating polymer units (A), (B), (C), (D) and (E), and also the optical or geometrical isomers thereof, the organic or mineral acid or base salts thereof, and also the solvates thereof:
—[—O—CH(R¹)—CH₂—C(O)—]— unit (A)
—[—O—CH(R²)—CH₂—C(O)—]— unit (B)
—[—O—CH(R³)—CH₂—C(O)—]— unit (C)
—[—O—CH(R⁴)—CH₂—C(O)—]— unit (D)
—[—O—CH(R⁵)—CH₂—C(O)—]— unit (E)
in which polymer units (A), (B), (C), (D) and (E):
R⁵ represents a saturated, linear or branched, cyclic or non-cyclic hydrocarbon-based chain comprising from 3 to 30 carbon atoms optionally substituted with one or more groups notably chosen from A) or B) as defined previously, optionally substituted with the groups a) to m) as defined for R¹ and/or optionally interrupted with one or more heteroatoms or groups a′) to c′) as defined for R¹ a) to m);
it being understood that:
(A) is different from (B), (C), (D) and (E); (B) is different from (A), (C), (D) and (E); (C) is different from (A), (B), (D) and (E); (D) is different from (A), (B), (C) and (E); and (E) is different from (A), (B), (C) and (D.

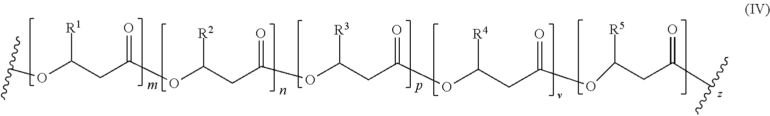

7. A polyhydroxyalkanoate PHA copolymer a) which contains the repeating unit of formula (I), as defined in claim 1, and also the optical or geometrical isomers thereof, the organic or mineral acid or base salts thereof, and the solvates thereof:

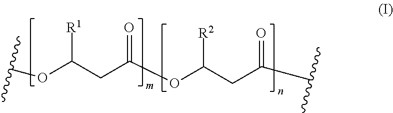

| Com- | | |
|---|---|---|
| pound | R¹ | R² |
| (1) | *-(ALK₁)-O—C(O)—CH₂—C(O)—CH₃ | -ALK₂ |
| (2) | *—(CH₂)ₚ—S-ALK₁-O—C(O)—CH₂—C(O)—CH₃ | -ALK₂ |
| (3) | *—(CH₂)ₚ—CH═CH-ALK₁-O—C(O)—CH₂—C(O)— | -ALK₂ |
| | CH₃ | |
| (5) | *—(CH₂)ₚ—CH(OH)—CH[C(O)—CH₃]—C(O)-R³ | -ALK₂ |
| (6) | *—(CH₂)ₚ—CH[C(O)—CH₃]—C(O)R³ | -ALK₂ |
| (7) | *—(CH₂)ₚ—S-ALK₁-O—C(O)—CH₂—C(O)—CH₃ | -ALK₂ |
| (8) | *—(CH₂)ₚ—CH═CH-ALK₁-O—C(O)—CH₂—C(O)— | -ALK₂ |
| | CH₃ | |
| (9) | *-(ALK₁)-O—C(O)—CH₂—C(O)—CH₃ | -ALK₂ |
| (10) | *—(CH₂)ₚ—S-ALK₁-O—C(O)—CH₂—C(O)—CH₃ | -ALK₂ |
| (11) | *—(CH₂)ₚ—S-ALK₃-[O—C(O)—CH₂—C(O)—CH₃]₂ | -ALK₂ |

where p is an integer between 3 and 15, ALK₁ denotes a divalent linear or branched C₁-C₁₅ hydrocarbon-based radical optionally substituted with a hydroxyl group, and R_{3′}, is as defined previously, ALK₂ denotes a C₃-C₂₀ alkyl radical; and ALK₃ denotes a trivalent linear or branched C₁-C₁₅ hydrocarbon-based radical, optionally substituted with a hydroxyl group, and R^{3′} is as defined previously,

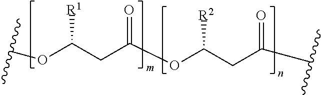

| Com- | | |
|---|---|---|
| pound | R¹ | R² |
| (1′) | *-(ALK₁)-O—C(O)—CH₂—C(O)—CH₃ | -ALK₂ |
| (2′) | *—(CH₂)ₚ—S-ALK₁-O—C(O)—CH₂—C(O)—CH₃ | -ALK₂ |
| (3′) | *—(CH₂)ₚ—CH═CH-ALK₁-O—C(O)—CH₂—C(O)— | -ALK₂ |
| | CH₃ | |
| (5′) | *—(CH₂)ₚ—CH(OH)—CH[C(O)—CH₃]—C(O)-R³ | -ALK₂ |
| (6′) | *—(CH₂)ₚ—CH[C(O)—CH₃]—C(O)R³ | -ALK₂ |
| (7′) | *—(CH₂)ₚ—S-ALK₁-O—C(O)—CH₂—C(O)—CH₃ | -ALK₂ |
| (8′) | *—(CH₂)ₚ—CH═CH-ALK₁-O—C(O)—CH₂—C(O)— | -ALK₂ |
| | CH₃ | |
| (9′) | *-(ALK₁)-O—C(O)—CH₂—C(O)—CH₃ | -ALK₂ |
| (10′) | *—(CH₂)ₚ—S-ALK₁-O—C(O)—CH₂—C(O)—CH₃ | -ALK₂ |
| [symbol] | [symbol] [symbol] | [symbol] |

8. The polyhydroxyalkanoate PHA copolymer a) according to claim 1, in which the radical(s) R¹ represent a hydrocarbon-based chain chosen from i) (C₅-C₂₂)alkyl, linear or branched ii) (C₅-C₂₂)alkenyl, linear or branched, said hydrocarbon-based chain being substituted with one or more groups chosen from A) R³—C(X)—C(R⁴)(R⁵)—C(X′)—[Y]ₙ—* with R³, R⁴, R⁵, X, X′, Y, n and * as defined previously.

9. The polyhydroxyalkanoate PHA copolymer a) according to claim 1, in which the radical R¹ represents a hydrocarbon-based chain chosen from i) linear or branched, (C₅-C₂₂)alkyl, ii) linear or branched, (C₅-C₂₂)alkenyl, said hydrocarbon-based chain being substituted, with one or more groups chosen from B) R³—C(X)—C(—[Y]ₙ—*)(R⁴)—C(X)—R⁶ with R³, R⁴, R⁶, X, X′, Y, n and * as defined previously.

10. The polyhydroxyalkanoate PHA copolymer a) according to claim 1, in which the radical R¹ represents a hydrocarbon-based chain chosen from i) linear or branched, (C₇-C₂₂)alkyl, ii) linear or branched, (C₇-C₂₂)alkenyl, said hydrocarbon-based chain being:
substituted, with a group chosen from A) and B); and
R³ and R⁶, which may be identical or different, representing a group chosen from (C₁-C₆)alkyl, (C₁-C₆)alkoxy;
R⁴ and R⁵, which may be identical or different, represent a hydrogen atom or a group chosen from (C₁-C₆)alkyl;
X and X′ are identical;
Y represents an oxygen atom;
* represents the point of attachment of the group A) or B) connected to the rest of the PHA copolymer(s); and
optionally substituted with one or more OH groups; and/or
optionally interrupted with one or more a′) heteroatoms chosen from O and S, b′) carbonyl, c′) and combinations of a′) with b′).

11. The polyhydroxyalkanoate PHA copolymer a) according to claim 1, in which the radical R¹ is of the following formula —(CH₂)ᵣ—X-(ALK)ᵤ-G with X representing O, S, N(Rₐ), ALK represents a linear or branched (C₁-C₁₀)alkylene chain, r represents an integer between 6 and 11 inclusive; u is equal to 0 or 1; and G represents a radical A) or B).

12. The polyhydroxyalkanoate PHA copolymer a) according to claim 1, in which R₁ represents a group chosen from:
i) *-(ALK₁)O—C(O)—CH₂—C(O)—R^{3′};
ii) *—(CH₂)ₚ—S-ALK₁-O—C(O)—CH₂—C(O)—R^{3′};
iii) *—(CH₂)ₚ—CH═CH-ALK₁-O—C(O)—CH₂—C(O)—R^{3′};
iv) *—(CH₂)ₚ—O-ALK₁-C(O)—CH₂—C(O)—R^{3′};
v) *—(CH₂)ₚ—CH(OH)—CH[C(O)—CH₃]—C(O)—R^{3′};
iv) *—(CH₂)ₚ—CH[C(O)—CH₃]—C(O)—R^{3′};
vi) *—(CH₂)ₚ —O—C(O)-ALK₁-C(O)—CH₂—C(O)—R^{3′} and
vii) *—(CH₂)ₚ—S-ALK₃-[O—C(O)—CH₂—C(O)—CH₃]₂
where p is an integer between 3 and 15, ALK₁ denotes a divalent linear or branched C₁-C₁₅ hydrocarbon-based radical optionally substituted with a hydroxyl group, ALK₃ denotes a trivalent linear or branched C₁-C₁₅ hydrocarbon-based radical, optionally substituted with a hydroxyl group; and R^{3′} is as defined previously.

13. The polyhydroxyalkanoate PHA copolymer a) according to claim 1, in which R² represents a hydrocarbon-based group chosen from linear or branched (C₃-C₂₈)alkyl, and linear or branched (C₃-C₂₈)alkenyl.

14. A process for preparing the PHA copolymer as claim 1:
either from PHA with an unsaturated hydrocarbon-based chain (a) which reacts with a nucleophilic reagent YH according to the following scheme 1 to lead to compound (b): 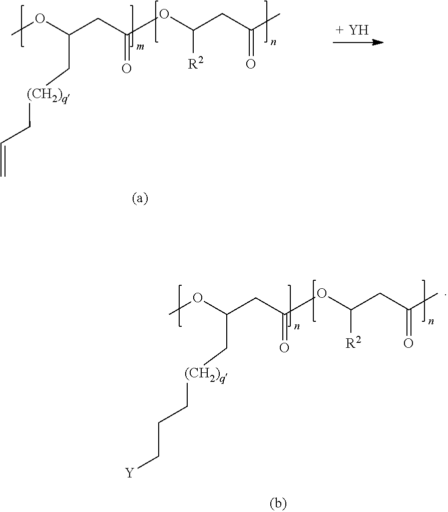
in which Scheme 1:
R², m and n are as defined previously;
Y represents a group chosen from R³—C(X)—C(R⁴)(R⁵)—C(X′)—[Y]ₙ-L-X″—* and R³—C(X)—C(—[Y]ₙ-L-X″—*)(R⁴)—C(X)—R⁶ with R³, R⁴, R⁵, R⁶, n, Y, X, X′ and * as defined previously, L representing a linear or branched, divalent hydrocarbon-based chain comprising from 1 to 20 carbon atoms with r being equal to 1 or 2, and R_{b} and R_{c} being as defined previously for Rₐ;
q′ represents an integer between 2 and 20 inclusive;
either from PHA with a hydrocarbon-based chain bearing an epoxide group (c) which reacts with a nucleophilic reagent YH according to the following scheme 2 to lead to compound (d): 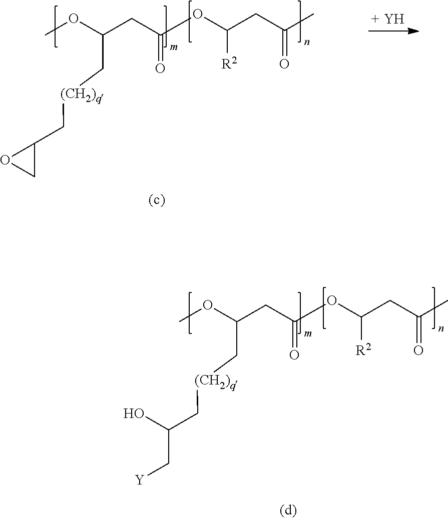
in which Scheme 2 Y, m, n, q′ and R² are as defined in Scheme 1;
or from PHA with a hydrocarbon-based chain bearing a nucleofugal group (e) which reacts with a nucleophilic reagent YH according to the following scheme 3 to lead to compound (f): 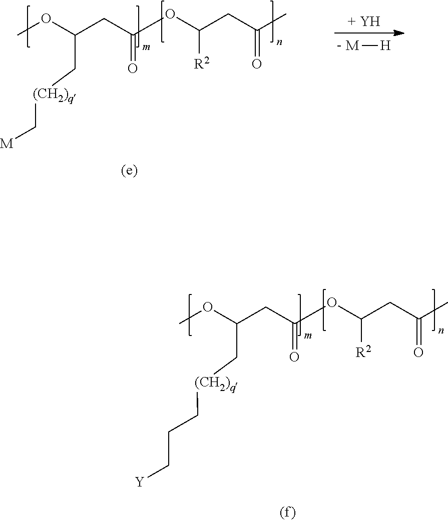
in which Scheme 3 Y, m, n, q′ and R² are as defined in Scheme 1; M corresponds to an organic or inorganic nucleofugal group, which may be substituted with a nucleophilic group;
or from PHA with a hydrocarbon-based chain bearing a cyano group which reacts with a nucleophilic reagent YH according to the following scheme 4: 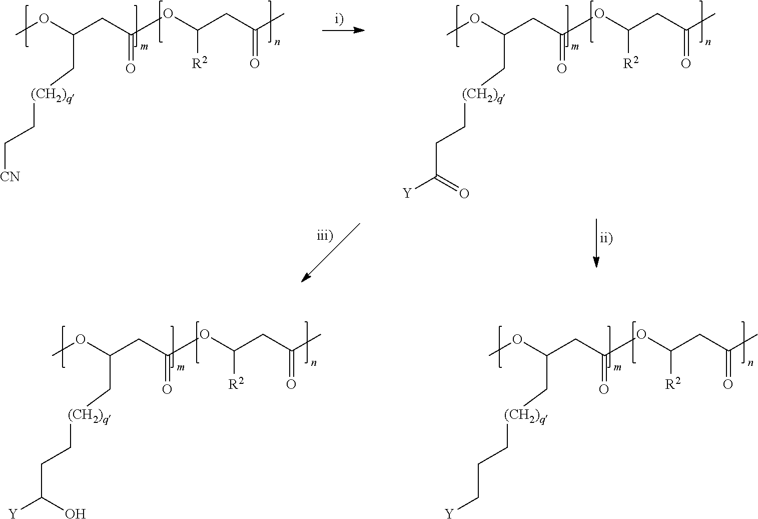
in which Scheme 4 Y, m, n, q′ and R² are as defined in Scheme 1;
in a first step i), the PHA copolymer bearing a side chain containing a cyano or nitrile group reacts with an organo-alkali metal or organomagnesium compound Y-MgHal, Y—Li or Y—Na, followed by hydrolysis to give the PHA copolymer bearing a side chain containing a group Y grafted with a ketone function, the ketone function may be changed to a thio ketone by thionation, which compound, after total reduction ii), leads to the PHA copolymer bearing a side chain containing a group Y grafted with an alkylene group; or
alternatively, said thio ketone may undergo a controlled reduction iii) with a conventional reducing agent to give the PHA copolymer bearing a side chain containing a group Y grafted with a hydroxyalkylene group;
or from PHA with a hydrocarbon-based chain at the end of the chain (g) which reacts with a reagent HR′¹ according to the following scheme 5 to lead to compound (h): 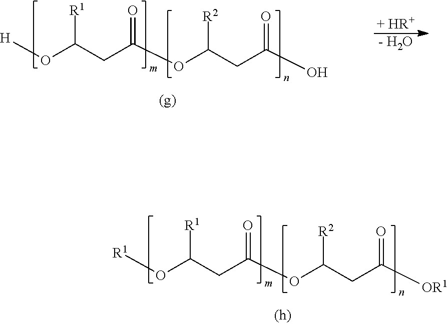
in which Scheme 5 R¹, R², m, n and Y are as defined previously, and R′¹ represents a hydrocarbon-based chain chosen from i) linear or branched (C₁-C₂₀)alkyl, ii) linear or branched (C₂-C₂₀)alkenyl, iii) linear or branched (C₂-C₂₀)alkynyl; said hydrocarbon-based chain being substituted with one or more atoms or groups chosen from: a) halogens such as chlorine or bromine, b) hydroxyl, c) thiol, d) (di)(C₁-C₄)(alkyl)amino, e) (thio)carboxyl, f) (thio)carboxamide —C(O)—N(Rₐ)₂ or —C(S)—N(Rₐ)₂, f) cyano, g) iso(thio)cyanate, h) (hetero)aryl, and i) (hetero)cycloalkyl, j) a cosmetic active agent chosen from coloured or uncoloured, fluorescent or non-fluorescent chromophores, or chromophores derived from UVA and/or UVB screening agents, and anti-ageing active agents, k) A) as defined previously, 1) B) as defined previously or m) Y as defined previously.
or from PHA with a hydrocarbon-based chain bearing a reactive group (i) which reacts with a nucleophile or an electrophile according to the following scheme 6 to lead to compound (j): 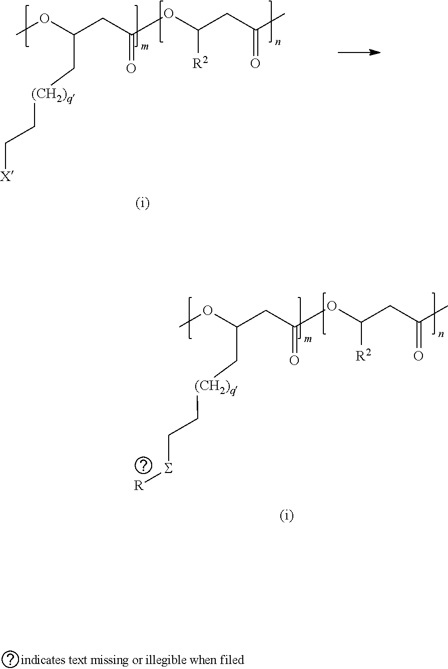
in which Scheme 6 R², m, n and Y are as defined previously, and R′¹ represents a hydrocarbon-based chain chosen from i) linear or branched (C₁-C₂₀)alkyl, ii) linear or branched (C₂-C₂₀)alkenyl, iii) linear or branched (C₂-C₂₀)alkynyl, said hydrocarbon-based chain being substituted with one or more groups chosen from A) as defined previously, B) as defined previously and Y as defined previously;
X′ represents a reactive atom or group that is capable of reacting with an electrophilic E or nucleophilic Nu atom or group to create a Σ covalent bond; if X′ is an electrophilic or nucleofugal group, then it can react with a reagent R′¹-Nu; if X′ is a nucleophilic group Nu, then it can react with R′¹-E to create a Σ covalent bond;
or from PHA ( ) which reacts at the end of the chain with a reagent HR′¹ according to the following scheme 7 to lead to compound (k): 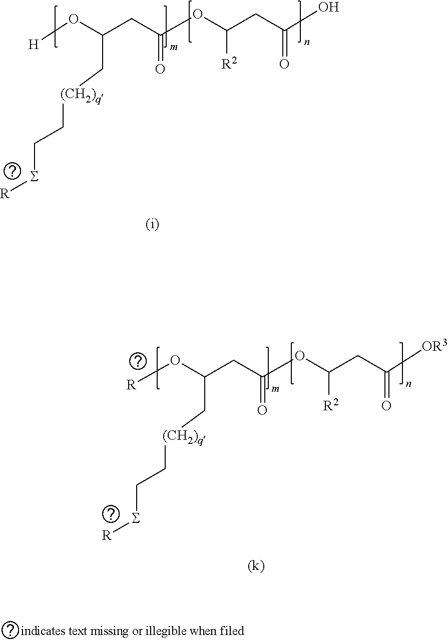
in which Scheme 7 R², m, n and Σ are as previously defined, and R′¹ represents a hydrocarbon-based chain chosen from i) linear or branched (C₁-C₂₀)alkyl, ii) linear or branched (C₂—C₂₀)alkenyl, iii) linear or branched (C₂-C₂₀)alkynyl, said hydrocarbon-based chain being substituted with one or more groups chosen from A) as defined previously, B) as defined previously and Y as defined previously.

15. A composition which comprises a) one or more PHAs as defined in claim 1.

16. The composition according to claim 15, which also comprises b) one or more crosslinking agent(s)

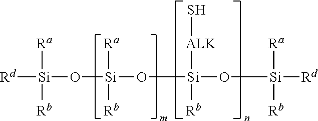

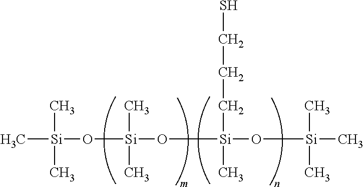

17. The composition according to claim 15, which comprises c) one or more fatty substances.

18. The composition according to claim 15, which comprises c) one or more fatty substances, which chosen from:
plant oils formed by fatty acid esters of polyols;
linear, branched or cyclic esters containing more than 6 carbon atoms O—R^{e} in which R^{d} represents a higher fatty;
hydrocarbons;
ethers containing 6 to 30 carbon atoms;
ketones containing 6 to 30 carbon atoms;
aliphatic fatty monoalcohols containing 6 to 30 carbon atoms, the hydrocarbon-based chain not including any substitution groups;
polyols containing 6 to 30 carbon atoms; and
mixtures thereof.

19. The composition according to claim 15, which also comprises one or more colouring agents chosen from pigments, direct dyes and mixtures thereof.

20. A process for treating keratin materials, by applying to the keratin materials one or more PHA copolymer(s) as defined in claim 1.

21. The process for treating keratin materials as defined in claim 20, which is performed in at least two successive steps:
in the first step (base coat):
a) a PHA copolymer(s) is applied to the keratin materials; and then;
in a second step (top coat):
b) a crosslinking agent(s) is applied to the keratin materials.

22. A process for making up the skin, or for colouring or styling keratin fibres which comprises applying to the skin or keratin fibres at least one PHA copolymer according to claim 1.

23. A device or kit with several separate compartments, comprising:
in a first compartment: a composition (Al) comprising:
a) one or more PHAs as defined in claim 1; and
optionally one or more fatty substances c); and/or
optionally one or more colouring agents; and
in a second compartment separate from the first: a composition (B1) comprising:
one or more crosslinking agents.
